(19) United States
(12) Patent Application Publication
(10) Pub. No.: US 2015/0174202 A1
(43) Pub. Date: Jun. 25, 2015

(54) Methods and Compositions for Modulating Beta Cell Proliferation

(71) Applicant: The Board of Trustees of the Leland Stanford Junior University, Palo alto, CA (US)

(72) Inventors: Hainan Chen, Cranford, NJ (US); Seung K. Kim, Stanford, CA (US)

(73) Assignee: The Board of Trustees of the Leland Stanford Junior University, Palo Alto, CA (US)

(21) Appl. No.: 14/347,540

(22) PCT Filed: Sep. 27, 2012
(86) PCT No.: PCT/US12/57602

§ 371 Date: Mar. 26, 2014

Related U.S. Application Data
(60) Provisional application No. 61/540,524, filed on Sep. 28, 2011.

(51) Int. Cl. A61K 38/18; A61K 31/713; A61K 38/02; C07K 16/22; A61K 31/365; A61K 31/7048; A61K 31/519; A61K 35/36; A61K 31/167

(52) U.S. Cl. CPC A61K 38/1858; A61K 35/36; A61K 31/713; A61K 38/02; A61K 31/167; A61K 31/365; A61K 31/7048; A61K 31/519; C07K 16/22

(57) ABSTRACT

Methods and compositions are provided for modulating pancreatic islet β cell proliferation. Aspects of the methods include promoting β cell proliferation by providing agents that promote PDGFR signaling, or inhibiting β cell proliferation by providing agents that inhibit PDGFR signaling. These methods find a number of uses, including, for example, in the treatment of diabetes and human islet diseases. Also provided are reagents and kits thereof that find use in practicing the subject methods.

### CROSS REFERENCE TO RELATED APPLICATIONS

Pursuant to 35 U.S.C. §119 (e), this application claims priority to the filing date of the U.S. Provisional Patent Application Ser. No. 61/540,524, filed Sep. 28, 2011; the disclosure of which is herein incorporated by reference.

### FIELD OF THE INVENTION

This invention pertains to modulating islet β cell proliferation.

### BACKGROUND OF THE INVENTION

Defects in β cell function and number underlie many human diseases. Emerging strategies to achieve replacement or regeneration of pancreatic β cells in the case of loss of β cell mass or function, or to attenuate growth in the case of β cell hypertrophy or hyperactivity, rely on tools to modulate β cell proliferation. The present invention addresses these issues.

### SUMMARY OF THE INVENTION

Methods and compositions are provided for modulating pancreatic islet β cell proliferation. Aspects of the methods include promoting β cell proliferation by providing agents that promote PDGFR signaling, or inhibiting β cell proliferation by providing agents that inhibit PDGFR signaling. These methods find a number of uses, including, for example, in the treatment of diabetes and human islet diseases. Also provided are reagents and kits thereof that find use in practicing the subject methods.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 illustrates age-dependent attenuation of Pdgfr-α limits β-cell Ezh2 expression and proliferation in neonatal and juvenile mice. a, Pdgfr-α and insulin immunostaining on wild-type mouse pancreas sections at indicated ages. DAPI, 4′,6-diamidino-2-phenylindole. Scale bar, 25 μm. b, c, mRNA levels of indicated genes in FACS-purified β-cells (b) or wild-type islets (c) at specified ages. n=3-5 experiments per group or time point. *P<0.05, **P<0.01 compared to samples from 2 weeks (b) or 15 days (c). Insr. refers to mRNA encoding insulin receptor. d-g, Relative mRNA levels of Pdgfra (d) and Ezh2 (e) in isolated islets, and percentage β-cell BrdU incorporation (f) and pancreatic β-cell mass (g) of βPdgfrαKO and control mice at specified ages. n=3-5 independent islet preparations or mice per group. *P<0.05, **P<0.01 for the comparison as indicated. Error bars denote s.e.m.

FIG. 2 illustrates how Pdgfra loss impairs β-cell regeneration in STZ-induced diabetes. a, Immunostaining of Pdgfr-α and insulin on pancreas sections from 8-week-old wild-type mice 1 week after vehicle or STZ (100 mgkg⁻¹ body weight) treatment. n=3-5 mice per group. Yellow arrows mark Pdgfr-α induction in a subset of insulin+ cells. b, Representative images showing immunostaining for insulin, Ezh2 and BrdU on pancreatic sections from indicated 6-7-week-old mice 3 weeks after STZ (150 mgkg⁻¹ body weight) treatment. c, Relative mRNA levels of Pdgfra and Pdgfrb in islets from 8-week-old wild-type mice 1 week after vehicle or STZ as depicted in (a). d, Percentage of BrdU+ insulin+ cells (β-cells) or BrdU+ acinar cells 3 weeks after STZ challenge. n=3 or 6 mice per group. e, f, Pancreatic β-cell mass (e) and blood glucose (f) levels during ad libitum feeding after STZ treatment. n=3-8 mice (e) or 18-21 mice (f) per time point per group. *P<0.05, **P<0.01 for the comparison as indicated or versus control. Scale bars, 25 μm. Error bars denote s.e.m.

FIG. 3 illustrates how activated PDGFR-α delays age-dependent Ezh2 loss and replication failure in pancreatic β-cells. a, Relative mRNA levels of human PDGFRA and mouse Pdgfra in islets from littermate control and βPDGFRαTg mice at 3 months of age. n=3-7 mice per group. b-d, Pancreatic β-cell mass (b), and β-cell proliferation, assessed by Ki67 expression (c) or BrdU incorporation (d) in βPDGFRαKO and control mice at indicated ages. n=3-5 mice per group. e, f, Relative mRNA levels of Ezh2 (e) in islets, and immunostaining (f) for Ezh2, BrdU and insulin on pancreatic sections from control and βPDGFRαTg mice at indicated ages. n=3-7 mice per group. Scale bars, 25 μm. g, Postprandial blood glucose levels in mice at indicated ages. n=14-24 mice per group per time point. h, Glucose tolerance assessed in 14-month-old control and βPDGFRαTg mice. n=6 or 10 mice per group. *P<0.05, **P<0.01 in comparisons indicated. Error bars denote s.e.m.

FIG. 4 illustrates how PDGFR-α promotes β-cell expansion through Ezh2. a, b, mRNA levels for human PDGFRA (a) and mouse Ezh2 (b) in islets from mice with indicated genotypes at 3-4 months of age. n=3-5 mice per group. c-f, β-Cell BrdU incorporation (c), β-cell mass (d), postprandial plasma insulin (e) and blood glucose (f) levels in 3-4-month-old mice with indicated genotypes. Each dot in f represents a measurement from an individual mouse. n=4-5 mice for c and d, and n=5-18 mice for e and f per genotype. *P<0.05, **P<0.01. NS, not significant in comparisons indicated. Error bars denote s.e.m.

FIG. 5 illustrates how Pdgfr signalling governs Erk and Rb/E2f regulation of Ezh2 in islet β-cells. a, Western blot analysis of total and phosphorylated Erk1/2, Akt and PLCγ in islet proteins from 3-4-month-old βPDGFRαTg and littermate control mice. Similar results were obtained from multiple independent experiments. b, Relative phosphorylated protein level compared to total protein was quantified by densitometry. c, Immunostaining for phospho-Erk1/2 (pErk1/2), phospho-Rb-Ser780 (p-Rb(Ser780)) and insulin in 3-month-old littermate control and βPDGFRαTg pancreatic sections. Scale bar, 25 μm. d, Percentage of phospho-Rb-Ser780⁺ insulin⁺ cells from the indicated mice quantified by morphometry. n=4 mice per group. e, Cyclin D1 (Ccnd1) or cyclin D2 (Ccnd2) mRNA levels in 3-month-old control and βPDGFRαTg islets. n=4 or 6 mice per group. f, g, ChIP analyses of the Ezh2 and β-actin (Actb) loci with antibodies to E2f1 (f) and E2f4 (g) using the indicated amplicons in 3-4-month-old littermate control and βPDGFRαTg islets. n=3-4 independent experiments per antibody with independent islet samples. *P<0.05, **P<0.01 for control versus βPDGFRαTg. Error bars denote s.e.m.

FIG. 6 illustrates how PDGFR-α regulates human β-cell EZH2 expression and proliferation. a, b, Representative images showing immunostaining for PDGFR-α (a), phospho-ERK1/2 (pERK1/2), phospho-RB-Ser780 (pRB(Ser780)), EZH2 (b) and insulin (a and b) on pancreatic sections from juvenile and adult human subjects. PDGFR-α was detected in juvenile β-cells (arrows) but not in adult β-cells (arrowheads). Scale bars, 25 μm. c-f, Assessment of effects on human juvenile or adult islets after exposure to PDGF-AA (50 ngml⁻¹) for 2 days, with or without Sunitinib (2 μM) or U0126 (10 μM) co-treatment. c, Islet EZH2 mRNA levels after the indicated treatments. n=3-5 independent experiments. d, EZH2 locus ChIP analysis with anti-E2F1 antibody or IgG in human juvenile islets. n=3-4 for E2F1, n=2 for IgG. e, f, Human islet β-cell proliferation changes after the indicated treatments. Average percentage of BrdU+ insulin+ cells (e) was quantified by morphometry from sectioned islets immunostained (f) for insulin (green), glucagon (white) and BrdU (red). n=3-6 independent experiments. Scale bar, 25 μm. g, Illustration summarizing how Pdgfr-α signalling regulates β-cell Ezh2 and proliferation by activating Erk/Rb/E2f pathways sensitive to the indicated inhibitors. *P<0.05, **P<0.01 as indicated. Error bars denote s.e.m.

FIG. 7 demonstrates gene expression in islets of mice of various ages. (a) Immunostaining of Pdgfr-β(white or red) and insulin (green) in pancreas islets from 3 day- and 6 month-old WT mice. (b-e) Real-time PCR-RT analyses of islet mRNA levels of the genes encoding Pdgfr-βreceptor (Pdgfrβ) and Pdgf ligands (Pdgfa, Pdgfb and Pdgfc) in C57BL/6 mice at the indicated ages. n=3 or 5 mice for each age group. * P<0.05, ** P<0.01 compared to samples from 2 weeks by ANOVA tests. Error bars denote s.e.m.

FIG. 8 demonstrates relative levels of mRNAs encoding the indicated receptors and transcription factors in C57BL/6 mouse islets at the indicated ages measured by quantitative real-time RT-PCR. The expression of these factors does not decline significantly in aging islets. n=3 or 5 mice for each age group. Error bars show s.e.m.

FIG. 9 demonstrates the molecular features and phenotypes of βPdgfraKO mice. (a) Representative images showing immunostaining for Pdgfr-α, Insulin, Ezh2 and BrdU on pancreatic sections from 2 week-old βPDGFRαKO and littermate control mice. In βPDGFRαKO islets Pdgfrα protein was barely detectable in insulin+ cells (yellow arrows), but unaffected in non-insulin+ cells (yellow arrow heads). White scale bar=25 μm. (b-d) Real-time RT-PCR analyses of islet mRNA levels of the indicated genes from control and βPdgfrαKO mice at 3 and 8 weeks of age. n=3-4 independent islet preparations for each group. (e) Blood glucose levels during ad libitum feeding at the indicated ages. n=9-22 mice per group. (f,g) Intraperitoneal glucose tolerance tests (ipGTT) performed in the mice at the indicated ages. n=4-12 mice for each group. *P<0.05, **P<0.01 vs. control or groups as indicated. Error bars show s.e.m.

FIG. 10 illustrates how PDGF-AA induces Ezh2 expression and proliferation in juvenile islets but not in adult islets. Real-time RT-PCR analyses of mRNAs encoding Ezh2 and Ezh1(a-b, d-f), and Western immunoblots of indicated islet proteins (c) from 3 week-(a-e) or 7-9 month-old (c,f) WT islets 2 days after exposure to PDGF-AA alone, or PDGF-AA plus RTK inhibitors Sunitinib or Vargatef (a-c, f), or to insulin (d), prolactin (e) at the indicated concentrations. (g) Assessment of β-cell proliferation, performed by immunostaining of insulin and BrdU, in 3 week- and 12 month-old WT islets exposed to PDGF-AA (50 ng/ml), without or with Sunitinib (2 μM) or Vargatef (5 μM) for two days. (h) β-cell proliferation quantified by percentage of all insulin+cells with BrdU incorporation from (g). n=3 to 5 independent experiments using separate islet preparations. White scale bar=25 μm. *P<0.05, **P<0.01 for comparison as indicated or vs. vehicle control (PDGF-AA concentration at 0 ng/ml). Error bars denote s.e.m.

FIG. 11 demonstrates the intact pancreatic islet architecture in βPDGFRαTg mice. Representative images showing immunostaining of PDGFR-α and phospho-PDGFR-α (a), Ki67 (b), insulin (a-c), and glucagon, somatostatin, and pancreatic polypeptide (Panc. polypeptide) (c) in pancreas islets from 1 week-, or 3-month-old βPDGFRαTg and littermate control mice. White scale bar=25 μm.

FIG. 12 demonstrates β-cell proliferation, systemic glucose control and insulin sensitivity in βPDGFRαTg mice. (a) ipGTT performed in mice at one-month of age. Pancreas weight (b), fasting (c) and randomly fed (d) blood glucose levels in βPDGFRαTg and littermate control mice ranging from 1 to 14 months of age. (e) Insulin tolerance test analyzed at 3 months of age in βPDGFRαTg and littermate control mice. Islet p16INK4a (f), p19Arf (g) and Ezh1(h) mRNA levels from control and βPDGFRαTg mice at the indicated ages. n=3 to 7 mice for each group. (i) Postprandial plasma insulin levels in mice at 3 months and 14 months of age (j) ipGTT performed in 3 month old mice. n618 mice for each group. P<0.05, ** P<0.01 vs. control or groups as indicated. Error bars denote s.e.m.

FIG. 13 illustrates how PDGFRα promotes β-cell expansion through Ezh2. Representative images showing immunostaining of Ezh2, BrdU and Insulin in pancreas sections from 3-4 month-old mice with the indicated genotypes. Scale bar=25 μm.

FIG. 14 illustrates how Erk inhibitor U0126 attenuates β-cell Ezh2 expression and proliferation induced by PDGF-AA. (a) Real-time RT-PCR analyses of Ezh2 mRNA levels in 3 week-old WT mouse islets 2 days after exposed to PDGF-AA, without or with inhibitors at the indicated concentrations. (b) Assessment of β-cell proliferation, quantified by percentage of all insulin+cells with BrdU incorporation, in 3 week-old WT mouse islets exposed to PDGF-AA and/or inhibitors as indicated for 2 pdays. n=3 to 5 independent experiments using separate islet preparations. * P<0.05, ** P<0.01 for comparison as indicated. Error bars denote s.e.m. black filled bars: PDGF-AA alone; open bars: PDGF-AA plus U0126; grey filled bars: PDGF-AA plus LY294002; striped bars: PDGF-AA plus U-73122.

FIG. 15 demonstrates the molecular features of βPDGFRαTg and βPdgfraKO mice. (a) Immunostaining of Cyclin D1, Cyclin D2 and Insulin on pancreas sections from 3-month-old βPDGFRαTg and littermate control mice. (b) Relative levels of mRNAs encoding Cyclin D1 (Ccnd1) or Cyclin D2 (Ccnd2) in islets from 3 week-old control and βPdgfraKO mice. n=3 mice for each group. Error bars denote s.e.m. Immunostaining to detect phospho-Erk1/2(p-Erk1/2), phospho-Rb-ser780 (p-Rb), and Insulin in pancreatic sections from 2 week-old littermate control and βPdgfraKO mice. n=3 or 5 mice per group. (a) and (c), White scale bar=25 μm.

FIG. 16 depicts two candidate E2F binding sites in a promoter-proximal region of the murine Ezh2 locus, a first that is 5′ of exon 1, and a second that is between exon 1 and exon 2. The highlighted consensus sequences are highly conserved among species as indicated. The relative position of amplicons (1, 2, and 3) used for ChIP assays is marked by white squares.

FIG. 17 illustrates the age-dependent decline of Erk/Rb/E2F signaling pathway in mouse pancreatic islets. (a) Immunostaining using antibodies against phospho-Erk1/2 (p-Erk), phospho-Rb-ser780 (p-Rb), and Insulin in pancreatic sections from WT type at the indicated ages. White scale bars=25 μm. (b) Quantification of phospho-Rb-ser780+Insulin+cells as a percentage of all Insulin+cells from (a). n=3-4 mice for each age group. (c-e) ChIP analysis of the Ezh2 locus and β-actin (Actb) locus using the indicated amplicons (summarized in FIG. 15) and anti-E2F1 antibody (c), anti-E2F4 antibody (d) and anti-IgG (e) in islets isolated from juvenile (2-3 weeks old) and adult (7-9 months old) WT mice. Three to four independent ChIP experiments were performed for each antibody using separate islet preparations. * P<0.05, ** P<0.01 for comparison of juvenile vs. adult, or compared to the value of 3 days old. Error bars denote s.e.m.

FIG. 18 illustrates how inactivation of retinoblastoma family members Rb, p130 and p107 enhances Ezh2 expression and promotes islet cell expansion in adult mice. (a) Representative images showing immunostaining detection of phospho-Rb-ser780 (p-Rb ser780)), Ezh2, BrdU and Insulin in pancreas sections from adult RbTriKO and littermate control mice eight days after exposure to tamoxifen (Tam). White scale bars=25 μm. (b-c) Relative mRNA levels for Rb, p130 (b), and Ezh2 (c) in islets from control and RbTriKO mice at indicated times after Tam administration. n=3 to 6 mice per group. (d-e) ChIP analysis of Ezh2 and Actb loci using the indicated amplicons and anti-E2F1 antibody (d) and anti-E2F4 antibody (e) in islets purified from control and RbTriKO mice 8 days after Tam injection. n=4 independent assays for each antibody. (f) Quantification of BrdU⁺Insulin⁺ cells as a percentage of all Insulin⁺ cells in RbTriKO and control mice eight days after Tam injection. n=4 mice for each group. (g) Pancreatic β-cell mass in adult RbTriKO and littermate control mice before and eight days after βgyTam treatment. n=3 to 5 mice for each group. (h) Blood glucose levels in littermate control and βPDGFαTg mice fed ad libitum following the indicated Tam exposure. n=14 or 15 mice for each group. * P<0.05, ** P<0.01, *** P<0.001 in comparisons to control or as indicated. Error bars denote s.e.m.

FIG. 19 demonstrates the effects of PDGF-AA and/or Sunitinib on human islets. (a) Real-time RT-PCR measurement of EZH1 mRNA levels from juvenile and adult human islets 2 days after exposure to PDGF-AA (50 ng/ml), and without or with exposure to Sunitinib (2 μM) or U0126 (10 μM) as indicated. Data are from n=3 to 5 independent experiments using islets from at least three donors for each group. Each experiment was performed with duplicate or triplicate islet samples. Error bars denote s.e.m. (b) Confocal images showing BrdU nuclei co-localized to insulin⁺ cells in juvenile islets exposed to PDGF-AA (50 ng/ml) for 2 days. Images (1 to 4) were serial pictures obtained by confocal imaging with a distance of 50 nM between optical sections. White scale bar=25 μm 25 μm. (c) Representative images showing immunostaining of PDX1 and BrdU in juvenile and adult human islets 2 days after exposure to Vehicle, or PDGF-AA (50 ng/ml). The majority of PDGF-AA stimulated proliferating islet cells were PDX1+cells (arrow heads).

FIG. 20 demonstrates two CpG islands upstream of the translational start site of the mouse Pdgfra gene, spanning the regions −9040/−9610 and −5662/−5054, as identified using the CpG tool of the UCSC genome browser (v257). We note that the more 3′ promoter-proximal CpG island at −5662/−5054 aligns well with regions associated with histone H3K4me1 and H3K4me3, marks of enhancers or cis-regulatory elements, with lesser overlap in the more 5′ CpG island at −9040/−9610. Remarkably, there are also two CpG islands within the −3,600 to +118 region of the human PDGFRA promoter-proximal region, and evidence that DNA methylation within the most 3′ CpG island regulates PDGFRA expression (Toepoel et al 2008).

FIG. 21 illustrates how dosage-dependent de-repression of PDGFRA is observed in human and mouse islets exposed to 5-aza-C.

FIG. 22 illustrates how studies of age-dependent Axin2 expression in staged mouse islets revealed abundant expression in neo-natal mice, followed by rapid reduction to nearly undetectable levels in adult islets (panel A), a pattern and tempo reminiscent of declining Pdgfra, Pdgfrab, Ezh2 levels, and β-cell proliferation (panels B-D; Chen et al 2009, 2011). Likewise, in mice harboring a ‘Top-Gal’ synthetic reporter gene of Wnt signaling activity, β-galactosidase reporter expression declines in islet β-cells with age (panels E-G).

FIG. 23 illustrates how the combination of R-Spondin followed by PDGF-AA exposure leads to induction of Ezh2 mRNA in cultured islets.

FIG. 24 illustrates the immunohistochemical detection of PDGFRα (green) re-expression by PDX1+ β-cells in adult islets exposed to R-Spondin.

FIG. 25 illustrates increased mRNA levels encoding PDGFRα and PDGFRβ in adult human islets (mean age >40 years) exposed for two days either to purified Wnt3a or R-Spondin.

FIG. 26 illustrates how PDGF-AA activation is sufficient to drive human β-cell replication in vivo. (A) Increased serum PDGF-AA levels in control NOD-scid mice implanted with a subcutaneous osmotic pump, primed with 4 micrograms (μg) of purified PDGF-AA. (B) Increase of INSULIN⁺ cell BrdU incorporation in grafts from young human donors (n=3 independent juvenile donors, three week PDGF-AA infusion, but not in engrafted adult human islets. (C) Immunohistochemical detection of PDGF-AA and age-dependent BrdU incorporation (red) by insulin-expressing cells (green) in engrafted juvenile and adult human islets.

### DETAILED DESCRIPTION OF THE INVENTION

Methods and compositions are provided for modulating pancreatic islet β cell proliferation. Aspects of the methods include promoting β cell proliferation by providing agents that promote PDGFR signaling, or inhibiting β cell proliferation by providing agents that inhibit PDGFR signaling. These methods find a number of uses, including, for example, in the treatment of diabetes and human islet diseases. Also provided are reagents and kits thereof that find use in practicing the subject methods. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

Methods and compositions are provided for modulating pancreatic islet β cell proliferation. By pancreatic islet β cells, it is meant functional β cells of the pancreatic islets, i.e. β-cells that are capable of responding to increased blood glucose and secreting appropriate levels of insulin to lower and balance the levels of glucose in the blood stream. By modulating the proliferation of β-cells it is meant modulating the replication, or growth, of β cells, that is, causing either an increase or a decrease in the number of islet β-cells produced by proliferation in vitro or in vivo. Modulating the proliferation of β cells finds many uses. For example, positive modulation of, or “promoting”, the proliferation/growth of β-cells may be used to increase the number of β cells in the body, e.g. in an individual with diabetes, which can increase insulin levels and return the body's glucose/insulin balance to normal levels to treat diabetes. As another example, negative modulation, or “suppression”, of the proliferation/growth of β-cells may be employed to decrease or halt the production of β cells in the body in, e.g., an individual with insulinoma (a tumor of the pancreas that is comprised of β-cells).

The pancreas serves two major functions: (i) the production of digestive enzymes, which are secreted by exocrine acinar cells and routed to the intestine by a branched ductal network; and (ii) the regulation of blood sugar, which is achieved by endocrine cells of the islets of Langerhans. Several separate endocrine cell types comprise the islet. Pancreatic β cells, also referred to as β-cells or “beta cells”, are the most prominent (50-80% of the total, depending on species); they produce a number of polypeptides including insulin, a hormone that controls the level of glucose in the blood; C-peptide, a byproduct of insulin production, which helps to prevent neuropathy and other symptoms of diabetes related to vascular deterioration; and amylin, also known as islet amyloid polypeptide (IAP, or IAPP), which functions as part of the endocrine pancreas and contributes to glycemic control. Glucagon-producing α-cells are the next most-common cell type. The remaining islet cells, each comprising a small minority of the total, include δ-cells, which produce somatostatin; PP cells, which produce pancreatic polypeptide; and ε-cells, which produce ghrelin.

The subject methods may be used to promote or suppress the proliferation of islet βcells. As discussed above, islet β cells store and release a number of factors into the body, As such, islet β cells may be readily distinguished from other cells of the pancreas by the presence of dense core secretory granules (DCGs) which contain proteins such as insulin and islet amyloid polypeptide, granins encoded by chromogranin A (ChgA) and chromogranin B (ChgB), and transmembrane proteins such as IA2 (also called ICA152). In addition, islet β cells express genes crucial for the production and secretion of insulin, including insulin 2, pancreatic duodenal homeobox 1 (Pdx1), type 2 glucose transporter (glut2), and glucokinase (Gck). Islet β cells in the expansion phase of islet proliferation, i.e. islet B cells from neonates or juveniles, or islet β cells from adults treated by the subject methods, also express certain known cell cycle regulators, including Ccnd1, Ccnd2, Cdk4, FoxM1, Ezh2, and PDGF receptors PDGFRα and PDGFRβ. By a neonate, it is meant, for example, a human of about 0-1 years of age, or a mouse of about 0-1 weeks in age. By a juvenile it is meant, for example, a human of about 1 year of age to about 12 years of age, e.g. 1-10 years old, 1-11 years old, 1-12 years old, 1-13 years old, or 1-14 years old; or a mouse of about 1 weeks of age to about 8 weeks age, e.g. 1-6 weeks old, 1-7 weeks old, 1-8 weeks old, 1-9 weeks old. Islet β cells from/in adults not treated by the subject methods, for example, an adult human of about 12 years of age or older, e.g. 11 years old or older, 12 years old or older, 14 years old or older, 16 years old or older, or 20 years old or older, or an adult mouse of about 8 weeks of age or older, e.g. about 6 weeks old or older, about 7 weeks old or older, about 8 weeks old or older, about 9 weeks old or older, about 10 weeks old or older, e.g. about 12 weeks old or older, express PDGF receptors at levels that are very low to undetectable.

In aspects of the subject methods, β cell proliferation is modulated by modulating the activity of the PDGFR pathway. PDGFRs (platelet-derived growth factor receptors) are cell surface tyrosine kinase receptors for platelet-derived growth factor (PDGF). There are two PDGF receptors in humans, each encoded by a different gene: PDGFRA, which encodes PDGFRα (“platelet-derived growth factor receptor, alpha”; Genbank Accession No. NM_{—}006206.4); and PDGFRB, which encodes PDGFRβ (“platelet-derived growth factor receptor, beta”; Genbank Accession No. NM_{—}002609.3). There are 5 known PDGF ligands in humans (PDGF-AA, PDGF-AB, PDGF-BB, PDGF-CC, PDGF-DD) created by the homo- or heterodimerization of PDGF polypeptides, each also encoded by a different gene: PDGF-A (“platelet-derived growth factor alpha polypeptide”, Genbank Accession Nos. NM_{—}002607.5 (isoform 1) and NM_{—}033023.4 (isoform 2)), PDGF-B (“platelet-derived growth factor beta polypeptide”, Genbank Accession Nos. NM_{—}002608.2 (isoform 1) and NM 033016.2 (isoform 2)), PDGF-C (“platelet derived growth factor C”, Genbank Accession No. NM_{—}016205.2), and PDGF-D (“platelet derived growth factor D”, Genbank Accession Nos. 1.NM_{—}025208.4 (isoform 1) and NM_{—}033135.3 (isoform 2)). Binding of PDGF ligand to PDGF receptor on the cell surface induces the PDGF receptor to homodimerize or heterodimerize, depending on which PDGF is bound. See, for example, Hoch and Soriano, Roles of PDGF in animal development. 2003. Development 130:4769-4784. Receptor dimerization induces receptor autophosphorylation at key tyrosine residues in the juxtamembrane domain, the kinase insert domain, and the C-terminal tail, which allows the receptor to bind to and phosphorylate intracellular signaling proteins to activate a number of signaling pathways including, for example, the Ras/mitogen-activated protein kinase (MAPK) pathway, e.g. shc, grb2, sos, ras, raf, mek, mek, erk1, erk2, Rb, E2F, etc.; the P-I3 kinase pathway, e.g. P-I3 kinase, PKB, akt, etc.; and the phospholipase-γ (PLCγ) pathway, e.g. shp1, shp2, PLC-gamma, PIP2, IP3, DAG, PKC, etc.

In some aspects of the subject methods, β-cell proliferation is promoted, and insulin secretion is increased. In such methods, β cells are contacted with an agent that promotes, i.e. enhances or augments, PDGFR signaling. Agents that promote PDGFR signaling include agents that activate or increase the expression of PDGFR, i.e. PDGFRA and/or PDGFRB. Agents that activate or increase the expression of PDGFR, i.e. PDGFRA and/or PDGFRB may be readily identified using any convenient technique for measuring gene expression, e.g. RT-PCR, Northern blot hybridization, Western blotting, etc.

For example, as demonstrated in the working examples below, the inventors have discovered that PDGFRA expression may be promoted, for example, induced (e.g. in a β cell of an adult that is no longer proliferating, e.g. in the presence of PDGF-AA) or enhanced (e.g. in a β cell of a neonate or juvenile that is still proliferating, e.g. in the presence of PDGF-AA), by contacting the β cell with an agent that promotes Wnt signaling.

By “Wnt protein signaling” or “Wnt signaling” is used herein to refer to the mechanism by which Wnt and R-Spondin proteins modulate cell activity. As is known in the art, Wnt proteins are member of the family of highly conserved secreted signaling molecules which play key roles in both embryogenesis and mature tissues. The human Wnt gene family has at least 19 members: Wnt-1 (RefSeq.: NM_{—}005430), Wnt-2 (RefSeq.: NM_{—}003391), Wnt-2B (Wnt-13) (RefSeq.: NM_{—}004185), Wnt-3 (ReSeq.: NM_{—}030753), Wnt3a (RefSeq.: NM_{—}033131), Wnt-4 (RefSeq.: NM_{—}030761), Wnt-5A (RefSeq.: NM_{—}003392), Wnt-5B (RefSeq.: NM_{—}032642), Wnt-6 (RefSeq.: NM_{—}006522), Wnt-7A (RefSeq.: NM_{—}004625), Wnt-7B (RefSeq.: NM_{—}058238), Wnt-8A (RefSeq.: NM_{—}058244), Wnt-8B (RefSeq.: NM_{—}003393), Wnt-9A (Wnt-14) (RefSeq.: NM_{—}003395), Wnt-9B (Wnt-15) (RefSeq.: NM_{—}003396), Wnt-10A (RefSeq.: NM_{—}025216), Wnt-10B (RefSeq.: NM_{—}003394), Wnt-11 (RefSeq.: NM_{—}004626), Wnt-16 (RefSeq.: NM_{—}016087). Although each member has varying degrees of sequence identity with the family, all encode small (i.e., 39-46 kD), acylated, palmitoylated, secreted glycoproteins that contain 23-24 conserved cysteine residues whose spacing is highly conserved (McMahon, A P et al., Trends Genet. 1992; 8: 236-242; Miller, J R. Genome Biol. 2002; 3(1): 3001.1-3001.15). As is known in the art, R-Spondin (RSPO) proteins are a family of secreted molecules that strongly potentiate Wnt/β-catenin signaling (see, e.g., Jin and Yoon (2012) The R-spondin family of proteins: Emerging regulators of WNT signaling. Int J Biochem Cell Biol. 2012 Sep. 13. pii: S1357-2725(12)00317-2). The human R-Spondin family has at least 4 members: R-Spondin 1 (RefSeq.: NM_{—}001038633.3 (isoform 1), RefSeq.: NM_{—}001242909.1 (isoform 2), RefSeq.: NM_{—}001242910.1 (isoform 3)); R-Spondin 2 (RefSeq.: NM_{—}178565.4); R-Spondin 3 (RefSeq.: NM_{—}032784.3); and R-Spondin 4 (RefSeq.: NM_{—}001029871.3 (isoform 1), RefSeq.: NM_{—}001040007.2 (isoform 2)).

Wnt/R-Spondin proteins modulate cell activity by binding to Wnt receptor complexes that include a polypeptide from the Frizzled (Fzd) family of proteins and a polypeptide of the low-density lipoprotein receptor (LDLR)-related protein (LRP) family of proteins. Fzd proteins are seven-pass transmembrane proteins (Ingham, P. W. (1996) Trends Genet. 12: 382-384; YangSnyder, J. et al. (1996) Curr. Biol. 6: 1302-1306; Bhanot, P. et al. (1996) Nature 382: 225-230). There are ten known members of the Fzd family (Fzd1 through Fzd10), any of which may be used in the Wnt receptor complex. LRP proteins are single-pass transmembrane proteins that bind and internalize ligands in the process of receptor-mediated endocytosis; LRP family members LRP5 (RefSeq.: NM_{—}002335.2) or LRP6 (RefSeq.: NM_{—}002336.2) are included in the Wnt receptor complex. Once activated by Wnt binding, the Wnt receptor complex will activate one or more intracellular signaling cascades. These include the canonical Wnt signaling pathway; the Wnt/planar cell polarity (Wnt/PCP) pathway; and the Wnt-calcium (Wnt/Ca²⁺) pathway (Giles, R H et al. (2003) Biochim Biophys Acta 1653, 1-24; Peifer, M. et al. (1994) Development 120: 369-380; Papkoff, J. et al (1996) Mol. Cell Biol. 16: 2128-2134; Veeman, M. T. et al. (2003) Dev. Cell 5: 367-377). For example, activation of the canonical Wnt signaling pathway results in the inhibition of phosphorylation of the intracellular protein β-catenin, leading to an accumulation of β-catenin in the cytosol and its subsequent translocation to the nucleus where it interacts with transcription factors, e.g. TCF/LEF, to activate target genes.

Any convenient agent that promotes Wnt signaling, i.e. signaling induced by Wnt and/or R-Spondin, e.g. as described here or known in the art, may be used to promote the expression of PDGFR in β cells in the subject methods. Non-limiting examples of such agents include a purified Wnt or active polypeptide thereof (e.g. a Wnt polypeptide precursor, a mature Wnt polypeptide in which the signal peptide sequence, e.g. as described in the RefSeqs provided above, has been removed, an active polypeptide fragment, e.g. as disclosed in PCT application Publication No. WO 2012/103360, the full disclosure of which is incorporated herein by reference); an R-spondin polypeptide or active polypeptide thereof (e.g. a R-spondin polypeptide precursor, a mature R-spondin polypeptide in which the signal peptide sequence, e.g. as described in the RefSeqs provided above, has been removed); a small molecule, e.g. WAY-316606 (Bodine et al. 2009. Bone 44(6):1063-8), IQ1 (Miyabayashi et al. 2007, Proc Natl Acad Sci USA. 104(13):5668-73), QS11 (Zhang et al. 2007. Proc Natl Acad Sci USA. 104(18):7444-8), SB-216763 (Coghlan et al. 2000. Chem Biol. 7(10):793-803), DCA (Pai et al. 2004. Mol Biol Cell. 15(5):2156-63), 2-amino-4-[3,4-(methylenedioxy)benzyl-amino]-6-(3-methoxyphenyl)pyrimidine (Liu et al. 2005, Angew Chem Int Ed Engl. 44(13):1987-90), etc.

Another example of an agent that activates or increases the expression of PDGFR is an agent that reduced the methylation of the PDGFR genomic locus. As demonstrated in the working examples below, the inventors have discovered that PDGFRA expression in a β cell may be activated or increased by contacting the β cell with an agent that inhibits the methylation of DNA. By “DNA methylation” or simply “methylation” it is meant the addition of a methyl group to DNA. In vertebrates, DNA methylation typically occurs on the nucleotide cytosine, usually at CpG sites (cytosine-phosphate-guanine sites; that is, where the cytosine is directly followed by a guanine in the DNA sequence). This results in the conversion of the cytosine to 5-methylcytosine, referred to interchangeably herein as “5-methylcytosine”, “5-meC”, and “methylated cytosine”. The added methyl group alters the structure of the cytosine without altering its base-pairing properties. DNA methylation promotes the recruitment of histone deacetylase (HDAC) complexes to the DNA, which promote DNA silencing. By an agent that inhibits the methylation of DNA it is meant an agent that inhibits the active of enzymes and cofactors, e.g. DNA methyltransferases, that catalyze the transfer of a methyl group to DNA, or an agent that promotes the activity of enzymes and cofactors, e.g. cytidine deaminases (CD), that promote the removal of methyl groups from DNA.

Any convenient agent that inhibits DNA methylation, that promotes DNA demethylation, or that inhibits the activity of HDACs may be employed in the subject methods. Nonlimiting examples of agents that inhibit DNA methylation include agents that inhibit the activity of DNA methyltransferases, e.g. DNMT1, DNMT2, or DNMT3, e.g. 5-aza-cytidine (5-aza-C, Vidaza), decitabine (Dacogen), and SGI110. Nonlimiting examples of agents that that promote the removal of methyl groups from DNA include agents that promote the activity of cytidine deaminases, e.g. Activation-induced Cytidine Deaminase (AID), and Apolipoprotein B RNA Editing Catalytic Component (APOBEC). Nonlimiting examples of agents that inhibit HDACs include hydroxamic acids (or hydroxamates), e.g. trichostatin A, vorinostat (SAHA), belinostat (PXD101), LAQ824, and panobinostat (LBH589); cyclic tetrapeptides, e.g. trapoxin B, and the depsipeptides; benzamides, e.g. entinostat (MS-275), CI994, and mocetinostat (MGCD0103); electrophilic ketones; and the aliphatic acid compounds, e.g. phenylbutyrate and valproic acid.

Agents that promote PDGFR signaling also include agents that bind to and activate PDGFRα or PDGFRβ, e.g. PDGFs. For example, as demonstrated in the working examples below, the inventors have discovered that β cell proliferation may be induced by contacting a PDGFR-expressing cell with PDGF-AA, i.e. a homodimer of PDGF alpha polypeptides. Other non-limiting examples of agents that promote PDGFR signaling by binding to PDGFR include other PDGF ligands as described above, e.g. PDGF-AB, PDGF-BB, PDGF-CC, and PDGF-DD, fragments of PDGF ligands that activate PDGFR, and activating antibodies that are specific for PDGFR. Agents that bind to and activate PDGFRα and/or PDGFRβ may be readily identified using any convenient cell-based or cell-free technique for measuring PDGFR binding, e.g. surface plasmon resonance, ELISA, immunohistochemistry, etc.

Agents that promote PDGFR signaling also include agents that promote the activity of proteins downstream of PDGFR, e.g proteins of the MAPK pathway, proteins of the P-I3 kinase pathway, proteins of the PLCγ pathway, proteins of the JAK/STAT pathway, etc. Such agents may be readily identified by assessing the activation state of these signaling proteins in a cell contacted with the agent, e.g. by detecting the phosphorylation state by, e.g. western blotting, flow cytometry, etc.

In other aspects of the subject methods, β cell proliferation is inhibited by contacting the β cells with an agent that antagonizes, i.e. suppresses, inhibits, attenuates, or negatively regulates, PDGFR signaling. Agents that antagonize PDGFR signaling include agents that suppress the expression of PDGFR, for example, siRNAs, e.g. PDGFRA-specific siRNA, PDGFRB-specific siRNA; Wnt antagonists, e.g. polypeptide inhibitors that bind to Wnt receptors or a subunit thereof, e.g. members of the sFRP (secreted Frizzled-related protein) family, members of the Dickkopf (Dkk) family, WIF (Wnt inhibitory factor)-1, and Cerberus, small molecule inhibitors of Wnt signaling, e.g. IWP (Chen et al 2009. Nat Chem Biol. (2):100-7), Ant1.4Br/Ant 1.4Cl (Morel) et al. 2008. PLoS One. 3(8):e2930.), niclosamide (Chen et al. 2009, supra), apicularen (Cruciate et al. 2010. Science 327(5964):459-63.), bafilomycin (Cruciat et al. 2010, supra), XAV939 (Huang et al., 2009. Nature 461(7264):614-20), IWR (Chen et al. 2009, supra), NSC668036 (Shan et al., 2005. Biochemistry 44(47):15495-503) 2,4-diamino-quinazoline (Chen et al. 2009, supra), quercetin (Park et al 2005. Biochem Biophys Res Commun 328(1):227-34), PKF115-584 (Lepourcelet et al 2004. Cancer Cell 5(1):91-102), etc.; and agents that promote the methylation of DNA or inhibit the demethylation of DNA. Such agents may be readily identified using methods such as those described above or known in the art.

Agents that antagonize PDGFR signaling also include agents that suppress the activation of PDGFR, for example by inhibiting the binding of PDGF to PDGFR. Examples of such agents include PDGF neutralizing antibodies, i.e. antibodies that bind to PDGF and inhibit binding of PDGF to PDGFR (e.g. 06-127 (Upstate) and ab10845 (Abcam), see Yamamoto et al. 2008. J Biol Chem 283(34):23139-49); PDGFR neutralizing antibodies, i.e. antibodies that bind to PDGFR and inhibit binding of PDGFR to PDGF (e.g. AF1062 (R&D Systems), see Taniguchi et al. 2008. Oncogene 27(51):6550); PDGFR peptide mimetics, i.e. a soluble polypeptide comprising the extracellular domain of PDGFRα, PDGFRβ, or a PDGF-binding fragment thereof that binds to PDGF and prevents PDGF binding to PDGFR; and the like. Such agents may be readily identified using methods such as those described above or known in the art.

Agents that antagonize PDGFR signaling also include agents that suppress PDGFR activity, e.g. PDGFR kinase activity on downstream proteins (that is, proteins that are either directly or indirectly activated by PDGFR activity), and the activity of these downstream proteins. Nonlimiting examples of small molecules that inhibit PDGFR activation of downstream proteins include Imatinib (Gleevac), Sunitinib Malate (Sutent), Axitinib, BIBF1120 (Vargatef), Pazopanib and Pazopanib HCl, Ponatinib (AP24534), MK-2461, Crenolanib (CP-868596), PP-121, Telatinib (BAY 57-9352), Amuvatinib (MP-470), TSU-68 (SU6668) and Motesanib Diphosphate (AMG-706), U0126, etc. Such agents may be readily identified using methods such as those described above or known in the art.

Any agent that modulates, i.e. promotes or antagonizes as described above, the PDGFR signaling pathway may be employed to modulate β cell proliferation in the subject methods. For example, small molecule compounds may be used. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992). Small molecule compounds can be provided directly to the medium in which the cells are being cultured, for example as a solution in DMSO or other solvent.

Another example of agents that modulate PDGFR signaling that would be suitable for use in the subject methods include nucleic acids, for example, nucleic acids that encode siRNA, shRNA or antisense molecules, or nucleic acids that encode polypeptides. Many vectors useful for transferring nucleic acids into target cells are available. The vector may be maintained episomally, e.g. as plasmid, minicircle DNA, virus-derived vector such as cytomegalovirus, adenovirus, etc., or it may be integrated into the target cell genome, through homologous recombination or random integration, e.g. retrovirus derived vectors such as MMLV, HIV-1, ALV, etc.

The nucleic acid agent may be provided directly to the β cells. In other words, the β cells are contacted with vectors comprising the nucleic acid of interest such that the vectors are taken up by the cells. Methods for contacting cells with nucleic acid vectors, such as electroporation, calcium chloride transfection, and lipofection, are well known in the art.

Alternatively, the nucleic acid agent may be provided to β cells via a virus. In other words, the β cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject β cells are targeted by the packaged viral particles. Methods of introducing the retroviral vectors comprising the nucleic acid of interest into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by 5 fold or more, by 10 fold or more, by at least about 100 fold or more, more usually by at least about 1000 fold. In addition, vectors used for providing nucleic acid to the subject cells may include genes that must later be removed, e.g. using a recombinase system such as Cre/Lox, or the cells that express them destroyed, e.g. by including genes that allow selective toxicity such as herpesvirus TK, bcl-xs, etc

Agents suitable for modulating PDGFR signaling in the present invention also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the polypeptide agent is to modulate PDGFR signaling intracellularly, the polypeptide may comprise the polypeptide sequences of interest fused to a polypeptide permeant domain. A number of permeant domains are known in the art and may be used in the polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the polypeptide agent is to modulate PDGFR signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

Stable plasma proteins are proteins which typically exhibit in their native environment an extended half-life in the circulation, i.e. greater than about 20 hours. Examples of suitable stable plasma proteins are immunoglobulins, albumin, lipoproteins, apolipoproteins and transferrin. The polypeptide agent typically is fused to the plasma protein, e.g. IgG at the N-terminus of the plasma protein or fragment thereof which is capable of conferring an extended half-life upon the polypeptide. Increases of greater than about 100% on the plasma half-life of the polypeptide are satisfactory. Ordinarily, the polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

The site at which the fusion is made may be selected in order to optimize the biological activity, secretion or binding characteristics of the polypeptide. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

The polypeptide agent for use in the subject methods may be produced from eukaryotic produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Another example of polypeptide agents suitable for modulating PDGFR signaling are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” The term “antibody” herein is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity. Antibodies are typically provided in the media in which the cells that produce them are cultured

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The modulator of PDGFR signaling activity (the “PDGFR signaling modulator”, or “PDGFR modulator”) is typically provided to cells in an effective amount, i.e. an amount that is effective to modulate PDGFR signaling and β cell proliferation. Biochemically speaking, an effective amount or effective dose of a PDGFR signaling modulator is an amount of modulator necessary to alter PDGFR signaling in a cell by 30% or more, 40% or more, 50% or more, 60% or more, 70% or more, 80% or more, 90% or more, 100% or more, 200% or more, or 500% or more. In other words, the activity of the PDGFR signaling pathway in a cell contacted with an effective amount or effective dose of a PDGFR signaling antagonist will be about 70% or less, about 60% or less, about 50% or less, about 40% or less, about 30% or less, about 20% or less, about 10% or less, about 5% or less, or will be about 0%, i.e. negligible, the activity observed in a cell that has not been contacted with an effective amount/dose of a PDGFR signaling antagonist, while the activity of the PDGFR signaling pathway in a cell contacted with an effective amount or effective dose of a PDGFR signaling agonist will be about 30% or more, 40% or more, 50% or more, 60% or more, 70% or more, 80% or more, 90% or more, 100% or more, 200% or more, or 500% or more. Put another way, PDGFR signaling will be altered about 0.5-fold or more, 1-fold or more, 2-fold or more, 5-fold or more, 8-fold or more, or 10-fold or more.

Suppressing β cell proliferation by the subject methods will produce a decrease in the rate of β cell proliferation, e.g. a decrease of 10% or more, of 20% or more, of 30% or more, of 40% or more, of 50% or more, sometimes a decrease of 60% or more, of 70% or more, in some instances a decrease of 80% or more, of 90% or more, for example, a cessation in proliferation of β cells. Conversely, promoting β cell proliferation by the subject methods is expected to promote a 2-fold increase or more in the number of β cells, e.g. a 3-fold increase or more, a 4-fold increase or more, a 5-fold increase or more, a 6-fold increase or more, in some instances, an 8-fold increase or more, a 10-fold increase or more, a 12-fold increase or more, for example, a 15-fold increase or more, a 20-fold increase or more, a 30-fold increase or more, in some instances, a 50-fold increase or more, in the number of β cells.

The extent to which PDGFR signaling and β cell proliferation is modulated by a PDGFR signaling modulator can be readily determined by a number of ways known to one of ordinary skill in the art of molecular biology. For example, changes in the level of expression of one or more genes known to be associated with proliferation in β cells, e.g. PDGFR, Ezh2, Ccnd2, FoxM1, and CcnA2 may be measured by RT-PCR, Northern Blot, or RNAse protection. As another example, the number, or mass, of β cells may be measured. For example, the number of β cells e.g. in culture or in a pancreatic biopsy may be counted by, e.g., flow cytometry or immunohistochemistry, or the number of new β cells may be counted by, e.g. BrdU labeling, before and after administration of the PDGFR signaling modulator and the change in β cell number calculated, where a greater increase in the number of β cells relative to the increase observed in culture or in a biopsy from an individual not contacted with PDGFR signaling modulator is indicative of increased PDGFR signaling and increased β cell proliferation. As another example, the amount of insulin, C-peptide, or IAPP, e.g. in the media of the culture dish or in the serum of the individual contacted by PGFR modulator may be quantified, e.g. by Western blot or ELISA. In these ways, the modulatory effect of the agent may be confirmed.

In a clinical sense, an effective dose of a PDGFR modulator is the dose that, when administered for a suitable period of time, usually at least about 3 days to about one week, and maybe about two weeks, or more, up to a period of about 4 weeks, 8 weeks, or longer will evidence an alteration the symptoms associated with β cell dysfunction or disorder. For example, an effective dose of an agent that promotes PDGFR signaling, i.e. a PDGFR agonist, is the dose that when administered for a suitable period of time, usually at least about one week, and may be about two weeks, or more, up to a period of about 4 weeks, 8 weeks, or longer will promote the proliferation of β cells resulting in, for example, a 2-fold increase or more in the number of β cells, e.g. a 3-fold increase or more, a 4-fold increase or more, a 5-fold increase or more, a 6-fold increase or more, in some instances, an 8-fold increase or more, a 10-fold increase or more, a 12-fold increase or more, for example, a 15-fold increase or more, or a 20-fold increase or more, in the number of β cells and production of insulin in a patient suffering from diabetes. As another example, an effective dose of an agent that suppresses PDGFR signaling, i.e. a PDGFR antagonist, is the dose that when administered for a suitable period of time, usually at least about one week, and may be about two weeks, or more, up to a period of about 4 weeks, 8 weeks, or longer will slow or halt the proliferation of β cells and maintain or reduce the production of insulin in a patient suffering from, e.g., insulinoma, mixed endocrine tumor, or acquired states of β cell overgrowth. It will be understood by those of skill in the art that an initial dose may be administered for such periods of time, followed by maintenance doses, which, in some cases, will be at a reduced dosage.

Calculating the effective amount or effective dose of PDGFR modulator to be administered is within the skill of one of ordinary skill in the art, and will be routine to those persons skilled in the art. Needless to say, the final amount to be administered will be dependent upon a variety of factors, include the route of administration, the nature of the disorder or condition that is to be treated, and factors that will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally or topically administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

In some embodiments, one agent is provided to the cells. For example, to induce proliferation in a β cell expressing PDGFR (e.g. a β cell from a neonate or juvenile), an agent that binds to and activates PDGFR, e.g. PDGF-AA, or an agent that activates proteins downstream of PDGFR may be provided to the cells. In other embodiments, more than one agent may be provided to the cells. For example, to induce proliferation in a β cell that does not express PDGFR (e.g. a β cell from an adult), and agent that promotes PDGFR expression, e.g. a purified Wnt, an R-spondin polypeptide, or a small molecule Wnt agonist, and an agent that promotes PDGFR activity, e.g. PDGF-AA, may be provided.

In some instances, the modulator of PDGFR signaling is used alone, i.e. in the absence of other growth factors, cytokines, etc, to promote or antagonize β cell proliferation. In some instances, the PDGFR signaling modulatory agent is used in combination with other agents, e.g. growth factors, cytokines, intracellular proteins, RNAs, small molecules, known in the art to modulate β cell proliferation, or function. For example, an agent that promotes PDGFR signaling may be used in combination with an agent known in the art to enhance the rate of β cell maturation, the number of β cells produced, or the production of insulin. Non-limiting examples of agents known in the art to promote β cell maturation, proliferation, and/or function include incretin and agents that promote incretin activity, e.g. Skp2 (Tschen et al. (2011) Skp2 is required for incretin hormone-mediated β-cell proliferation. Mol Endocrinol. 25(12):2134-43), and glucokinase activators and/or exendin-4 (Nakamura et al. (2012) Control of beta cell function and proliferation in mice stimulated by small-molecule glucokinase activator under various conditions. Diabetologia 55(6):1745-54), the full disclosures of which are incorporated herein by reference.

In some instances, the β cells that are induced to proliferate by the subject methods are in vivo. In other instances, the β cells are in vitro, for example, β cells that have been acutely purified from an individual, or β cells that have been induced to differentiate from progenitor cells, e.g. pluripotent stem cells, pancreatic progenitor cells, or endocrine progenitor cells, or somatic cells, e.g. pancreatic duct cells, fibroblasts, etc. in vitro. Methods for inducing the differentiation of progenitor cells into β cells or the transdifferentiation of somatic cells into β cells are well known in the art. See, for example, Goodyer W R, et al. Neonatal β Cell Development in Mice and Humans Is Regulated by Calcineurin/NFAT. Dev Cell. 23(1):21-34, and U.S. Application Ser. No. 61/670,813, the disclosures of which are incorporated herein in their entirety by reference. Typically, the cell is a postnatal cell.

### In Vitro Applications

The subject methods may be used to modulate β cell proliferation in vitro, for example to produce an enriched population of β cells, i.e. a population of cells that is enriched for β cells, for research or for transplantation into an individual.

Cells may be from any mammalian species, e.g. murine, rodent, canine, feline, equine, bovine, ovine, primate, human, etc. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

If the cells are primary cells, they may be harvest from pancreas by biopsy. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The modulator of PDGFR signaling is provided to the β cells in culture for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The modulator may be provided to the β cells one or more times, e.g. one time, twice, three times, or more than three times, and the cells allowed to incubate with the agent for some amount of time following each contacting event e.g. 16-24 hours, after which time the media is replaced with fresh media and the cells are cultured further.

Contacting the β cells with the modulator of PDGFR signaling may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which β cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Exemplary conditions may be found in the working examples provided below.

In some instances, the population of cells may be enriched for β cells by separating the β cells from the remaining population. Separation of β cells typically relies upon the expression of a selectable marker. By a “selectable marker” it is meant an agent that can be used to select cells, e.g. a marker that is ectopically provided, or a marker that is endogenously expressed by and specific for β cells. In some instances, the selection may be positive selection; that is, the β cells are isolated from a population, e.g. to create an enriched population of β cells. In other instances, the selection may be negative selection; that is, the population is isolated away from the β cells, e.g. to create an enriched population of cells that do not comprise the β cells.

Separation may be by any convenient separation technique appropriate for the selectable marker used. For example, if a fluorescent marker has been introduced into the cells, e.g. as progenitor cells, or during the course of differentiation, cells may be separated by fluorescence activated cell sorting. Alternatively, known markers of β cells, e.g. as described herein or known in the art, may be used. β cells may be separated from the heterogeneous population by affinity separation techniques, e.g. magnetic separation, affinity chromatography, “panning” with an affinity reagent attached to a solid matrix, flow cytometry, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the β cells.

For example, to separate the β cells by affinity separation techniques, cells that are not β cells may be depleted from the population by contacting the population with affinity reagents that specifically recognize and selectively bind markers that are not expressed on β cells. Additionally or alternatively, positive selection and separation may be performed using by contacting the population with affinity reagents that specifically recognize and selectively bind markers associated with β cells. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody. In some embodiments, the affinity reagent may be a specific receptor or ligand for a protein expressed on the cell surface e.g. a peptide ligand and receptor; effector and receptor molecules; a T-cell receptor, and the like. In some embodiments, multiple affinity reagents may be used. Markers and flow cytometry gating strategies that may be used to selectively purify β cells from other cells in the culture; see, for example Hald J, et al. ((2012) Pancreatic islet and progenitor cell surface markers with cell sorting potential. Diabetologia. 55(1):154-65); Szabat M, et al. ((2011) Kinetics and genomic profiling of adult human and mouse β-cell maturation. Islets. 3(4):175-87); and Köhler M, et al. ((2012) One-step purification of functional human and rat pancreatic alpha cells. Integr Biol (Camb). 4(2):209-19), the disclosures of which are incorporated herein in their entirety by reference.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The population comprising β cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent, i.e. the β cells, are selected for by any convenient affinity separation technique, e.g. as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Cell compositions that are highly enriched for β cells are achieved in this manner. By “highly enriched”, it is meant that the β cells will be 70% or more, 75% or more, 80% or more, 85% or more, 90% or more of the cell composition, for example, about 95% or more, or 98% or more of the cell composition. In other words, the composition may be a substantially pure composition of β cells.

β cells produced by the methods described herein may be used immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. The β cells may be cultured in vitro under various culture conditions. The cells may be further expanded in culture, i.e. grown under conditions such as those described herein that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Compositions of β cells that have been prepared by the subject methods find many uses. For example, such compositions may be used in research, e.g. to develop a better understanding of the nature of pancreatic diseases, or to screen candidate agents for those that may be developed to treat pancreatic disease, as described in greater detail below. As another example, such compositions may be transplanted to a subject for purposes such as to treat disease, e.g. diabetes. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals, e.g. murine, lagomorpha, etc. may be used for experimental investigations.

In some cases, the β cells may be genetically altered prior to transplanting to the individual, in order to introduce genes useful in the β cell, e.g. repair of a genetic defect in an individual, to provide a selectable or traceable marker, etc. The β cells may also be genetically modified to enhance survival, control proliferation, and the like. Cells may be genetically altering by transfection or transduction of the β cell with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the β cells, various techniques may be employed. The genome of the cells may be restricted and used with or without amplification. The polymerase chain reaction; gel electrophoresis; restriction analysis; Southern, Northern, and Western blots; sequencing; or the like, may all be employed. Various tests in vitro and in vivo may be employed to ensure that β cell phenotypes have been maintained.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the pancreas, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint or organ; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

The number of administrations of treatment to a subject may vary. Introducing the β cells into the subject may be a one-time event; but in certain situations, such treatment may elicit improvement for a limited period of time and require an on-going series of repeated treatments. In other situations, multiple administrations of the β cells may be required before an effect is observed. The exact protocols depend upon the disease or condition, the stage of the disease and parameters of the individual subject being treated.

### In Vivo Applications

The subject methods may also be used to modulate β cell proliferation in vivo, for example to augment the number or function of β cells in an individual, e.g. an individual with diabetes, or, for example, to suppress the expansion of β cells in an individual, e.g. an individual with insulinemia, In these in vivo embodiments, the modulator of PDGFR signaling is administered directly to the individual. A PDGFR signaling modulator may be administered by any of a number of well-known methods in the art and described below for the administration of peptides, small molecules and nucleic acids to a subject.

As discussed above, the modulator of PDGFR signaling is typically administered in an effective amount. The amount administered varies depending upon the goal of the administration, the health and physical condition of the individual to be treated, age, the taxonomic group of individual to be treated (e.g., human, non-human primate, primate, etc.), the degree of resolution desired, the formulation of the PDGFR signaling modulator composition, the treating clinician's assessment of the medical situation, and other relevant factors. It is expected that the amount will fall in a relatively broad range that can be determined through routine trials. For example, the amount of modulator of PDGFR signaling employed to promote β cell proliferation is not more than about the amount that could otherwise be irreversibly toxic to the subject (i.e., maximum tolerated dose). In other cases the amount is around or even well below the toxic threshold, but still in an effective concentration range, or even as low as threshold dose.

Individual doses are typically not less than an amount required to produce a measurable effect on the subject, and may be determined based on the pharmacokinetics and pharmacology for absorption, distribution, metabolism, and excretion (“ADME”) of the PDGFR signaling modulator or of its by-products, and thus based on the disposition of the composition within the subject. This includes consideration of the route of administration as well as dosage amount, which can be adjusted for topical (applied directly where action is desired for mainly a local effect), enteral (applied via digestive tract for systemic or local effects when retained in part of the digestive tract), or parenteral (applied by routes other than the digestive tract for systemic or local effects) applications. For instance, administration of the modulator of PDGFR signaling may be via injection, e.g. intravenous, intramuscular, or intrapancreatic injection, or a combination thereof.

The modulator of PDGFR signaling may be administered by infusion or by local injection, e.g. by infusion at a rate of about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m²/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration (e.g, by infusion) can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove β cells, e.g. in the case of β cell hypertrophy. The modulator of PDGFR signaling can also be administered as part of a combination therapy, in which at least one of an immunotherapy, a diabetes therapy, a cancer therapy, etc. also is administered to the subject (as described in greater detail below).

Disposition of the modulator of PDGFR signaling and its corresponding biological activity within a subject is typically gauged against the fraction of modulator of PDGFR signaling present at a target of interest. For example, a modulator of PDGFR signaling once administered can accumulate with a glycoconjugate or other biological target that concentrates the material in cancer cells and cancerous tissue. Thus dosing regimens in which the modulator of PDGFR signaling is administered so as to accumulate in a target of interest over time can be part of a strategy to allow for lower individual doses. This can also mean that, for example, the dose of PDGFR signaling modulator that are cleared more slowly in vivo can be lowered relative to the effective concentration calculated from in vitro assays (e.g., effective amount in vitro approximates mM concentration, versus less than mM concentrations in vivo).

As an example, the effective amount of a dose or dosing regimen can be gauged from the IC₅₀ of a given antagonist of PDGFR signaling for inhibiting β cell differentiation. By “IC₅₀” is intended the concentration of a drug required for 50% inhibition in vitro. Alternatively, the effective amount can be gauged from the EC₅₀ of a given PDGFR signaling modulator concentration. By “EC₅₀” is intended the plasma concentration required for obtaining 50% of a maximum effect in vivo. In related embodiments, dosage may also be determined based on ED₅₀ (effective dosage).

In general, with respect to the modulator of PDGFR signaling of the present disclosure, an effective amount is usually not more than 200× the calculated IC₅₀. Typically, the amount of a modulator of PDGFR signaling that is administered is less than about 200×, less than about 150×, less than about 100× and many embodiments less than about 75×, less than about 60×, 50×, 45×, 40×, 35×, 30×, 25×, 20×, 15×, 10× and even less than about 8× or 2× than the calculated IC₅₀. In one embodiment, the effective amount is about 1× to 50× of the calculated IC₅₀, and sometimes about 2× to 40×, about 3× to 30× or about 4× to 20× of the calculated IC₅₀. In other embodiments, the effective amount is the same as the calculated IC₅₀, and in certain embodiments the effective amount is an amount that is more than the calculated IC₅₀.

An effect amount may not be more than 100× the calculated EC₅₀. For instance, the amount of a modulator of PDGFR signaling that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC₅₀. The effective amount may be about 1× to 30× of the calculated EC₅₀, and sometimes about 1× to 20×, or about 1× to 10× of the calculated EC₅₀. The effective amount may also be the same as the calculated EC₅₀ or more than the calculated EC₅₀. The EC₅₀ can be calculated by modulating β cell proliferation in vitro. The procedure can be carry out by methods known in the art or as described in the examples below.

Effective amounts of dose and/or dose regimen can readily be determined empirically from assays, from safety and escalation and dose range trials, individual clinician-patient relationships, as well as in vitro and in vivo assays such as those described herein and illustrated in the Experimental section, below. For example, if a concentration used for carrying out the subject method in mice ranges from about 1 mg/kg to about 25 mg/kg based on the body weight of the mice, an example of a concentration of the PDGFR signaling modulator that can be employed in human may range about 0.083 mg/kg to about 2.08 mg/kg. Other dosage may be determined from experiments with animal models using methods known in the art (Reagan-Shaw et al. (2007) The FASEB Journal 22:659-661).

The PDGFR signaling modulator can be incorporated into a variety of formulations. More particularly, the PDGFR signaling modulator may be formulated into pharmaceutical compositions by combination with appropriate pharmaceutically acceptable carriers or diluents. Pharmaceutical preparations are compositions that include one or more PDGFR signaling modulator present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the PDGFR signaling modulator can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release. For inclusion in a medicament, the PDGFR signaling modulator may be obtained from a suitable commercial source. As a general proposition, the total pharmaceutically effective amount of the PDGFR signaling modulator administered parenterally per dose will be in a range that can be measured by a dose response curve.

PDGFR signaling modulator-based therapies, i.e. preparations of PDGFR signaling modulator(s) to be used for therapeutic administration, may be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The PDGFR signaling modulator-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection. Alternatively, the PDGFR signaling modulator may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The modulator of PDGFR signaling may be provided in addition to other agents. For example, in methods of inhibiting β cell proliferation, e.g. to treat insulinoma, mixed endocrine tumor, or acquired states of β cell overgrowth, a PDGFR signaling antagonist may be coadministered with other known cancer therapies. As another example, in methods of promoting β cell proliferation, e.g. to treat diabetes, a PDGFR signaling agonist may be coadministered with other known diabetes therapies.

### Utility

The subject methods and compositions find many uses. For example, the subject methods may be used to treat diseases associated with defective β cell maturation, proliferation, or function. The terms “treatment”, “treating” and the like are used herein to generally mean obtaining a desired pharmacologic and/or physiologic effect in an individual. The terms “individual,” “subject,” “host,” and “patient,” are used interchangeably herein and refer to any mammalian subject for whom diagnosis, treatment, or therapy is desired, particularly humans. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its progression; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

For example, the subject methods may be used to generate β cells ex vivo for replacement therapy, or to promote β cell proliferation in vivo to regenerate β cell mass, e.g. to treat disorders associated with a decrease in β cell mass or function. One example of a disease that may be treated in this way is diabetes. Diabetes is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”).

Type 1 diabetes, also known as insulin dependent diabetes mellitus (IDDM), results from the destruction or dysfunction of β cells by the cells of the immune system. Symptoms include polyuria (frequent urination), polydipsia (increased thirst), polyphagia (increased hunger), and weight loss. T1 D is fatal unless treated with insulin and must be continued indefinitely, although many people who develop the disease are otherwise healthy and treatment need not significantly impair normal activities. Exercising regularly, eating healthy foods and monitoring blood sugar may also be recommended. Other medications may be prescribed as well, including one or more of the following: medications to slow the movement of food through the stomach (e.g. pramlintide), high blood pressure medications, cholesterol-lowering drugs.

Type 2 diabetes, also known as non-insulin dependent diabetes mellitus (NIDDM), is associated with a gradual decline in β cell function and numbers over time, as the β cells develop resistance to insulin. As a result, in T2D the pancreas does not make enough insulin to keep blood glucose levels normal. Symptoms include hyperglycemia (high blood sugar), diabetic ketoacidosis (increased ketones in urine), and hyperosmolar hyperglycemic nonketotic syndrome. Therapy may include blood sugar monitoring; healthy eating; regular exercise; diabetes medication that lowers glucose production (e.g. metformin, sitagliptin, saxagliptin, repaglinide, nateglinide, exenatide, liraglutide), that stimulates the pancreas to produce and release more insulin (e.g. glipizide, glyburide, glimepiride), and/or that blocks the action of enzymes that break down carbohydrates or make tissues more sensitive to insulin (e.g. pioglitazone); and insulin therapy.

The subject methods find use in treating diabetes, e.g. diabetes type I or diabetes type II, for example by delivering a composition, either externally or internally, to a patient with the disease that will reduce the progression of diabetes, maintain the level of diabetes, and/or reverse the symptoms of diabetes in a patient. By progression of diabetes it is meant the increase in malfunctioning of the glucose/insulin imbalance in a patient, and in particular the decrease in the presence or functioning of β-cells in the islets of Langerhans. Likewise, by reverse the symptoms of diabetes in a patient it is meant causing a decrease in the disease symptoms and an increase in the patient's control of the glucose/insulin balance. By “glucose/insulin balance,” it is meant the body's ability to respond to glucose increases or decreases in the blood by modulating the insulin level, so as to maintain a healthy level of glucose in the bloodstream.

Other disorders associated with insulin resistance that may likewise be treated by promoting β cell proliferation using the subject methods include, for example, diabetic angiopathy, atherosclerosis, diabetic nephropathy, diabetic neuropathy, and diabetic ocular complications such as retinopathy, cataract formation and glaucoma, as well as glucocorticoid induced insulin resistance, dyslipidemia, polycysitic ovarian syndrome, obesity, hyperglycemia, hyperlipidemia, hypercholesterolemia, hypertriglyceridemia, hyperinsulinemia, and hypertension.

As another example, the subject methods may be used to treat disorders associated with increased β-cell mass or β cell hyperactivity, for example by inhibiting β cell proliferation in vivo to prevent further hypertrophy or hyperactivity. Examples of diseases that may be treated in this way include congenital or acquired hyperinsulinism, nesidioblastosis following bariatric surgery, insulinomas and other neuroendocrine cancers.

Hyperinsulinism refers to an above-normal level of insulin in the blood of a person or animal. In normal children and adults, insulin secretion should be minimal when blood glucose levels fall below 70 mg/dL (3.9 mM). Insulin levels above 3 μU/mL are inappropriate when the glucose level is below 50 mg/dL (2.8 mM), and may indicate hyperinsulinism as the cause of the hypoglycemia. There are many forms of hyperinsulinemia caused by various types of insulin excess. For example, congenital hyperinsulinism occurs in infants and young children, and may be the result of genetic abnormalities, the intrauterine environment, errors of morphogenesis, infection, or a chromosomal abnormality. In adults, severe hyperinsulinemia is often due to an insulinoma (an insulin-secreting tumor of the pancreas, discussed further below). Hyperinsulinemia may also be caused by nesidioblastosis, e.g. after bariatric (e.g. gastric bypass) surgery. Treatment of hyperinsulinism depends on the cause and the severity of the hyperinsulinism, and may include surgical removal of the source of insulin, or a drug such as diazoxide or octreotide that reduces insulin secretion.

Insulinoma is a rare tumor derived from β cells. Insulin secretion by insulinomas is not properly regulated by glucose. As such, tumors continue to secrete insulin, causing glucose levels to fall further than normal. The diagnosis of an insulinoma is usually made biochemically with low blood glucose, elevated insulin, proinsulin and C-peptide levels and confirmed by localizing the tumor with medical imaging or angiography. The definitive treatment is surgery. Insulinomas are usually benign and not malignant, but may be medically significant and even life-threatening due to recurrent and prolonged attacks of hypoglycemia. Insulinomas and other neuroendocrine cancers would therefore benefit from treatment using the subject methods.

Methods for modulating β cell proliferation by providing a PDGFR signaling modulator may also be applied to studying β cell proliferation, and/or β cell function in vitro. For example, the methods described above provide a useful system for screening candidate agents for activity modulating β cell proliferation. To that end, it has been shown that PDGFR signaling modulates β cell proliferation. Accordingly, screening candidate agents to identify those that promote PDGFR activity should identify agents that find use in promoting β cell proliferation, whereas screening candidate agents to identify those that inhibit PDGFR activity should identify agents that find use in inhibiting β cell proliferation. In one example of such a screen, β cells are contacted with a candidate agent, and one or more cellular parameters reflective of the activity of the PDGFR signaling pathway is measured. The measured cellular parameter(s) are compared to the cellular parameter(s) measured in β cells not contacted with the candidate agent. An increase in PDGFR activity indicates that the candidate agent will promote β cell proliferation.

As another example, screening candidate agents to identify those that promote β cell proliferation in PDGFR-knockout cells should identify signaling pathways other than the PDGFR signaling pathway that promote β cell proliferation and that can be targeted for drug development, whereas screening candidate agents to identify those that inhibit β cell proliferation in the presence of an activator of PDGFR signaling should identify signaling pathways other than the PDGFR signaling pathway that inhibit β cell proliferation and that can be targeted for drug development. In one example of such a screen, PDGFRA knockout β cells, e.g. as described in the working examples below or as known in the art, are contacted with a candidate agent under conditions that normally promote β cell proliferation, and one or more cellular parameters reflective of β cell proliferation is measured. The measured cellular parameter(s) are compared to the cellular parameter(s) measured in PDGFRA KO cells not contacted with the candidate agent. An increase in β cells in the culture indicates that the candidate agent targets a protein that promotes β cell proliferation independent of PDGFRA.

Cellular parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. As will be readily apparent to the ordinarily skilled artisan, a number of output cellular parameters may be quantified when screening for agents that modulate the activity of PDGFR, or that modulate the proliferation of β cells. For example, the phosphorylation of PDGFR or of targets downstream of PDGFR, e.g. proteins of the MAPK signaling pathway, Rb, etc. may be assessed by, e.g., western blotting. The expression of target genes, e.g. genes for which expression is increased upon PDGFR-mediated cell activation, may be measured, e.g. by Northern blot, RT-PCR, Western blot, etc. Parameters reflective of the extent of β cell proliferation in the culture may be measured, e.g. by assessing BrdU incorporation, by counting the total number of β cells in the culture, by detecting the amount of insulin, C-peptide, or IAPP produced by the cells, etc. Any convenient parameter that reflects the activity of PDGFR signaling and/or an increase in the number of β cells in the culture may be measured. In some instances, multiple parameters are measured.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Nucleic acids may be provided as vectors, viruses, or any other convenient method known in the art or described elsewhere herein.

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of biochemical or cell-based reactions, usually in conjunction with biochemical reactions or cells not contacted with the agent. The change in parameters in response to the agent is measured, and the result evaluated by comparison to reference samples, e.g. in the presence and absence of the agent, obtained with other agents, etc. The agents are conveniently added in solution, or readily soluble form, to the cell-free reaction or medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The subject compositions comprising β cells prepared by the subject methods may also be used as a basic research or drug discovery tool, for example to evaluate the phenotype of a genetic disease, e.g. to better understand the etiology of the disease, to identify target proteins for therapeutic treatment, to identify candidate agents with disease-modifying activity, i.e. an activity in modulating the survival or function of β cells in a subject suffering from a pancreatic disease or disorder, e.g. to identify an agent that will be efficacious in treating the subject.

### Reagents and Kits

Also provided are reagents and kits thereof for practicing one or more of the above-described methods. The subject reagents and kits thereof may vary greatly. Reagents and devices of interest include those mentioned above with respect to the methods of promoting or inhibiting β cell proliferation, treating disorders such as diabetes or insulinoma that are associated with defects in β-cell maturation, proliferation or function, and screening candidate agents for the ability to treat disorders associated with defects in β cell maturation, proliferation or function by modulating PDGFR signaling to modulate β cell proliferation, proliferation, and function. Reagents may include one or more of the following: one or more agents that is an agonist of PDGFR signaling, e.g. purified Wnt, R-spondin, 5-aza-C, PDGF, etc., or one or more agents that is an antagonist of PDGFR signaling, e.g. Sunitinib, Vargatef, PDGFR blocking antibodies, etc.; and buffer or pharmaceutical excipient into which the agent(s) may be dissolved for contacting cells or administering to an individual. In some kits, e.g. kits for performing screens, kits for analyzing the function of β cells, etc., cells, media, and reagents as discussed above or in the working examples below may also be provided.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

As used herein, the name of a gene is shown in italics while the protein produced from that gene is shown in regular font. For example, Ezh2 is a gene that codes for Ezh2 protein.

The addition of the letter “r” or “R” at the end of a gene name, or “r” or “R” at the end of a protein name means that the gene codes for a receptor of the protein expressed from the gene without the attached r. For example, Pdgfr codes for the protein Pdgfr that is the receptor for the protein coded for by the gene Pdgf. Likewise, the protein Pdgfr is the receptor for the protein Pdgf. Acronyms that are in all capital letters, such as PDGF, refer to human proteins from the PDGF gene. Acronyms that have an initial capital letter followed by lower case letters, such as Pdgf, refer to mouse analogs of the human proteins, from the Pdgf gene.

Pdgfr signalling promotes proliferation, survival and migration in diverse cell types (Schlessinger, J. Cell signaling by receptor tyrosine kinases. Cell 103, 211-225 (2000)). Pdgf signalling activation stimulates DNA synthesis in cultured islets (Swenne, I. et al. Effects of platelet-derived growth factor and somatomedin-C/insulin-like growth factor I on the deoxyribonucleic acid replication of fetal rat islets of Langerhans in tissue culture. Endocrinology 122, 214-218 (1988); Welsh, M. et al. Coexpression of the platelet-derived growth factor (PDGF) B chain and the PDGF beta receptor in isolated pancreatic islet cells stimulates DNA synthesis. Proc. Natl Acad. Sci. USA 87, 5807-5811 (1990)), but the roles of Pdgf signalling in β-cell proliferation and expansion were unknown. The data present in the examples below demonstrate that a pathway regulated by Pdgfr-α governs the expression and activity of intrinsic β-cell factors—including the retinoblastoma protein (Rb), E2f transcription factors and Ezh2—to control β-cell proliferation. Inducing Pdgf receptor expression in adult β-cells, and promoting β-cell Pdgf receptor signalling in neonate, juvenile and adult β-cells, attenuates age-dependent proliferative failure and Ezh2 loss, permitting in vivo expansion of β-cells with regulated function. Cardinal features of this pathway are conserved in human islets, permitting conditional induction of human β-cell replication.

### Materials and Methods

Animals.

Transgenic mice harbouring a floxed Pdgfra allele (Tallquist, M. D. & Soriano, P. Cell autonomous requirement for PDGFRα in populations of cranial and cardiac neural crest cells. Proliferation 130, 507-518 (2003)) encoding Pdgfr-α were purchased from the Jackson Laboratory. This strain of mice was intercrossed with RIP-Cre mice (Herrera, P. L. Adult insulin- and glucagon-producing cells differentiate from two independent cell lineages. Proliferation 127, 2317-2322 (2000)) expressing Cre recombinase from rat insulin 2 gene promoter elements to generate RIP-Cre, Pdgfra^{f/f} mice (designated as βPdgfrαKO) and their littermate controls (including RIP-Cre, Pdgfrα^{f/+} mice designated as βPdgfrαHet) on a mixed 129/Sv; C57BL/6 genetic background.

R26-hPDGFRα^{PM} transgenic mice that harbour a mutated human PDGFRA^{D842V} allele encoding a ligand-independent activated receptor targeted immediately after a loxP-flanked transcriptional stop sequence at the ROSA26 locus have been described previously. This strain of mice was intercrossed with RIP-Cre mice to generate RIP-Cre; R26-PDGFRA^{D842V} mice (designated as βPDGFRαTg) together with their littermate controls on a mixed 129/Sv; C57BL/6 genetic background. Mice with specific expression of mutant human PDGFRA^{D842V}, but lacking Ezh2 in β-cells (designated as βEzh2KO-RαTg), were generated from subsequent intercrosses between βEzh2KO13 and βPDGFRαTg mice.

Tamoxifen-inducible, compound triple-mutant mice lacking retinoblastoma pocket proteins Rb, p130 and p107 (ROSA26-CreERT2; Rb^{f/f}; p130^{f/f}; p107^{-/-}, designated as RbTriKO) were produced in the Sage group (Stanford University). RbTriKO mice harbour a germline-deleted p107 null allele (p107^{-/-}), and loxP-flanked Rb and p130 conditional alleles which can be excised by tamoxifen-sensitive CreERT2 recombinase targeted into the ROSA26 locus. Littermate mice (Rb^{f/f}; p130^{f/f}; p107^{-/-} lacking ROSA26-CreERT2 were used as controls. To delete Rb and p130 alleles, RbTriKO and littermate control mice aged 3-5 months received intraperitoneal injections of tamoxifen (Sigma, 1.5 mg per mouse per day) dissolved in ethanol/corn oil (Sigma) on five consecutive days.

MIP-EGFP mice42 were obtained from the laboratory of M. Hara. C57BL/6 mice were purchased from the National Cancer Institute (NCI). All mutant mice used in this study were genotyped by PCR of tail genomic DNA for the human PDGFRA^{D842V} allele, mouse Pdgfraf, Ezh2f, Rbf, p130f, p107− alleles, and transgenes encoding EGFP, RIP-Cre or ROSA-CreERT2 as described previously (Chen et al. Polycomb protein Ezh2 regulates pancreatic β-cell Ink4a/Arf expression and regeneration in diabetes mellitus. Genes Dev. 23, 975-985 (2009); Moenning, A. et al. Sustained platelet-derived growth factor receptor α signaling in osteoblasts results in craniosynostosis by overactivating the phospholipase C-γ pathway. Mol. Cell. Biol. 29, 881-891 (2009); Viatour, P. et al. Hematopoietic stem cell quiescence is maintained by compound contributions of the retinoblastoma gene family. Cell Stem Cell 3, 416-428 (2008); Tallquist, M. D. & Soriano, P. Cell autonomous requirement for PDGFRα in populations of cranial and cardiac neural crest cells. Proliferation 130, 507-518 (2003); Herrera, P. L. Adult insulin- and glucagon-producing cells differentiate from two independent cell lineages. Proliferation 127, 2317-2322 (2000); Hara, M. et al. Transgenic mice with green fluorescent protein-labeled pancreatic β-cells. Am. J. Physiol. Endocrinol. Metab. 284, E177-E183 (2003)). Mice used in this study were age- and gender-matched littermates including both sexes.

Physiological Studies.

Glucose physiology studies were performed as reported in Chen et al. Polycomb protein Ezh2 regulates pancreatic β-cell Ink4a/Arf expression and regeneration in diabetes mellitus. Genes Dev. 23, 975-985 (2009). Briefly, at the ages or times indicated in the text and figures, or after tamoxifen administration, we monitored body weight and blood glucose levels in ad libitum fed mice, or after 16 h overnight fasting, or 2 h after re-feeding (‘2 h postprandial’). We drew blood from tail vein punctures or from the aorta when mice were killed, for measuring blood glucose levels using a Contour glucose meter (Bayer), or for measuring plasma insulin levels by mouse insulin ELISA kits (ALPCO Diagnostics).

Glucose tolerance tests were performed on mice after a 16 h overnight fast, and the blood glucose levels were determined immediately before (0) and 5, 10, 20, 30, 45, 60, 90 and 120 min after intraperitoneal injection of glucose (2 g kg⁻¹ body weight). For measurement of plasma insulin levels during these glucose tolerance tests, mice were injected with glucose at a dose of 3 g kg⁻¹ body weight, and we collected tail vein blood at 0, 15 and 45 min after glucose injection followed by mouse insulin ELISA (ALPCO diagnostics). For insulin tolerance tests, mice were fasted for 6 h then received intraperitoneal injection of 0.75 units kg⁻¹ of body weight of insulin (Sigma); glucose levels at 0, 10, 20, 30, 40, 60 and 90 min after injection were then measured.

For detecting in vivo β-cell proliferation by BrdU incorporation, mice were either fed with water containing BrdU (1 mg ml⁻¹) or intraperitoneally injected with BrdU/PBS solution (50 mg kg⁻¹ body weight) for specified schedules as follows: βPdgfraKO mice, 2 days of intraperitoneal injection of BrdU/PBS; βPdgfraKO mice with STZ treatment, 1 week of BrdU water feeding; βPDGFRαTg or βEzh2KO-RαTg mouse studies, 6-7 days of BrdU water feeding; RbTriKO mice, 4 days of BrdU water feeding. After BrdU chase, mouse pancreases were collected and subjected to immunohistology followed by morphometric analyses described later.

STZ-Induced Diabetes.

For measuring islet Pdgfra and Pdgfrb induction during STZ-induced diabetes, male wild-type C57BL/6 mice, aged 8 weeks, were injected intraperitoneally with 100 mg kg⁻¹ body weight of freshly dissolved streptozotocin (STZ, Sigma) in 0.1 mol I⁻¹ sodium citrate (pH 4.5) or with the same volume of sodium citrate (Vehicle). Six days later, islets or pancreases were recovered from the mice for gene expression analyses by real-time RTPCR or immunohistology studies. To test the roles of Pdgfra in islet regeneration in STZ diabetes, βPdgfrαKO or control mice (including βPdgfrαHet), aged 7-8 weeks, were treated with a single intraperitoneal STZ injection at a dose of 150 mg kg⁻¹ body weight. After injection, blood glucose values were measured on day 3 and day 6 in the first week, and thereafter once per week. After 2 weeks of STZ treatment, subsets of βPdgfrαKO and control littermates were fed with BrdU-containing drinking water (1 mg ml⁻¹) for 1 week for BrdU incorporation analyses. Three days, 21 days and 58 days after STZ treatment, subsets of mice were killed, and blood samples and pancreases were harvested for measurement of plasma insulin levels by ELISA, and for pancreatic β-cell proliferation and mass quantification following immunostaining of insulin and BrdU.

Islet Isolation and Culture Studies.

Mouse pancreatic islets were purified as described in Chen et al. (2009) Genes Dev. 23, 975-985 and used immediately for assays described in the text. To study Ezh2 induction in cultured islets exposed to mitogens, freshly isolated islets from 2-3-week old, and 7-12-month old C57BL/6 mice were recovered and equilibrated overnight at 37° C., 5% CO₂ in RPMI-1640 medium supplemented with 0.5% fetal bovine serum (FBS), 0.2% bovine albumin (BSA) and 2% penicillin/streptomycin. Afterwards, islets were distributed into microplates or dishes, and treated with recombinant human PDGF-AA, prolactin or insulin (Sigma), with or without addition of pharmacological inhibitors, including Sunitinib (Selleck), Vargatef (Selleck), U0126 (Sigma), LY294002 (Sigma) or U-73122 (Sigma) at the indicated concentrations. Two days later, islets were harvested for RNA or protein extraction for real-time RTPCR or western blot analyses. To detect phosphorylation of Pdgfr-α by western blot, islets were exposed to PDGF-AA (with or without indicated pharmacological inhibitors) for 90 min before islet harvesting and protein extraction. To detect in vitro islet β-cell proliferation stimulated by PDGF-AA, a final concentration of 50 μM BrdU was introduced into each culture dish 24 h before harvest, followed by standard histological processing. Immunostaining of BrdU and insulin was performed on processed islets, and β-cell proliferation rate was determined by quantifying percentage of insulin+ cells with BrdU+. Culture experiments of human islets were performed using a similar procedure described later.

β-Cell Purification by FACS.

Freshly purified islets from MIP-GFP mice at 2 weeks or 5 months of age were pooled, and dispersed into a suspension of single cells by incubation with 0.05% trypsin/0.53 mM EDTA solution at 37° C. for 10 min. After dissociation, cells were washed three times in PBS containing 2% FBS, and re-suspended in FACS buffer (HBSS/0.2% BSA/1% HEPES) containing propidium iodide (to exclude dead cells). FACS was performed on a FACS Ariall (BD Biosciences), and MIP-GFP+β-cells with >95% purity were harvested and immediately lysed for RNA purification and real-time RT-PCR analysis.

Real-Time RTPCR and Western Blot Analysis.

Total RNA from freshly isolated or cultured islets after indicated treatments were purified using the Absolutely RNA miniprep purification kit (Stratagene) according to the manufacturer's instructions. RNA concentration was measured with a RiboGreen RNA quantification assay (Invitrogen). One-step quantitative RT-PCR was performed and analysed using an ABI Prism 7300 detection system (Applied Biosystems) with TaqMan one-step RTPCR Master Mix Reagents and appropriate amounts (10-100 ng) of islet total RNA as the template. We calculated the ratio of mRNA for the gene of interest to the amount of internal control mRNA of peptidylprolyl isomerase A (cyclophilin A, PPIA), and then normalized the ratio for each gene to its median. Primer and probe sequences are listed in Tables 1 and 2, below.

For western blots, total islet protein was prepared from freshly isolated or cultured islets after indicated treatments as described in Chen et al. (2009) Genes Dev. 23, 975-985. Equal amounts of protein were resolved on SDS-PAGE and transferred to polyvinylidine fluoride membranes (Amersham Pharmacia) for immunoblotting with specific antibodies, including rabbit polyclonal anti-human Ezh2 (1:1,000, Millipore), rabbit polyclonal anti-phospho-PDGFR-α (Tyr 720) (1:1,000, Santa Cruz Biotechnologies), rabbit monoclonal anti-PDGFR-α (1:1,000, Cell Signaling), mouse monoclonal anti-β-actin (1:4,000, Sigma), rabbit monoclonal anti-Akt (1:1,500, Cell Signaling), rabbit monoclonal anti-phospho-Ark (Ser473) (1:1,000, Cell Signaling), rabbit monoclonal anti-p44/42 MAPK (Erk1/2) (1:1,500, Cell Signaling), rabbit monoclonal anti-phospho-p44/42 MAPK (Erk1/2) (Thr202/Tyr204) (1:1,000, Cell Signaling), rabbit monoclonal anti-PLCγ1 (1:1,000, Cell Signaling) and rabbit monoclonal anti-phospho-PLCγ1 (Tyr783) (1:1,000, Cell Signaling). Signal was visualized using ECL detection (Amersham Pharmacia) on Kodak film after further incubation with HRP-conjugated secondary antibodies. For phospho-PDGFR-α (Tyr720) and PDGFR-α detection, signal was amplified by incubating the membrane with biotinylated anti-rabbit-IgG (1:2,000), followed with an incubation with HRP-avidin (1:2,000, Vector Laboratories) and then ECL visualization.

TABLE 1
Custom designed qRT-PCR Primers used for mouse and
human islet mRNA expression assays.

| Gene | Forward (5′-3′) | Probe (5′-3′) | Reverse (5′-3′) |
|---|---|---|---|
| Ezh2 | GACAAATACATGTG | CAACTTGAACAATGATT | GCCCTTTCGGGTTGC |
| | CAGCTTTCTGT | TTGTGGTG (SEQ ID | AT (SEQ ID NO: 3) |
| | (SEQ ID NO: 1) | NO: 2) | |
| Rb1 | GAGCTTGGCTAACT | TCCGTGGATGGAATCC | ATGCAGATGCCCCAG |
| | TGGGAGAA (SEQ ID | TGGAAGGAT(SEQ ID | AGTTC (SEQ ID NO: 6) |
| | NO: 4) | NO: 5) | |
| p130 | GCAGCTACCGCAG | CGAGAGCTACACGCTG | AAGGCACATGCTAAC |
| | CATGA (SEQ ID | GAGGGAAATG (SEQ ID | CAATGAA (SEQ ID |
| | NO: 7) | NO: 8) | NO: 9) |
| p107 | GCAACTACAGCCTA | TACACTGGCTGGCATG | CTCTTGCGGCAAGCA |
| | GAGGGAGAAGT | CTCTTT | ACATA (SEQ ID NO: 12) |
| | (SEQ ID NO: 10) | (SEQ ID NO: 11) | |
| p16INK4a | GTACCCCGATTCAG | CGTTCACGTAGCAGCT | CAGTTCGAATCTGCA |
| | GTGATGA (SEQ ID | CTTCTGC (SEQ ID | CCGTAGT (SEQ ID |
| | NO: 13) | NO: 14) | NO: 15) |
| p₁₉Arf | TGAGGCTAGAGAG | CCGGAATCCTGGACCA | CGTGAACGTTGCCCA |
| | GATCTTGAGAAG | GGTGA | TCAT (SEQ ID NO: 18) |
| | (SEQ ID NO: 16) | (SEQ ID NO: 17) | |
| Ppia | TGCTGGACCAAACA | TTCCCAGTTTTTTATCT | TGCTTGCCATCCAGC |
| | CAAACG (SEQ ID | GCACTG (SEQ ID | CATTCA (SEQ ID |
| | NO: 19) | NO: 20) | NO: 21) |
| EZH2 | TTGCCAAGAGAGCC | ACTGGCGAAGAGCTGT | GCATCAGCCTGGCTG |
| | ATCCA (SEQ ID | TTTTTGA (SEQ ID | TATCTG (SEQ ID |
| | NO: 22) | NO: 23) | NO: 24) |
| PPIA | ATAAGGGTTCCTGC | TCCAGGGTTTATGTGTC | GCCATTATGGCGTGT |
| | TTTCACAGAA (SEQ | AGGGTG (SEQ ID | GAAGTC (SEQ ID |
| | ID NO: 25) | NO: 26) | NO: 27) |

TABLE 2
List of qRT-PCR assay IDs used for mouse and human islet
mRNA expression assays purchased from Applied Biosystems.

| Gene | | | |
|---|---|---|---|
| Symbol | Gene Name | Species | Assay ID |
| PDGFRA | Platelet-derived growth | Human | Hs00998029_m1 |
| | factor receptor, alpha | | |
| | polypeptide | | |
| Pdgfra | Platelet derived growth | murine | Mm00440701_m1 |
| | factor receptor, alpha | | |
| | polypeptide | | |
| Pdgfrb | Platelet derived growth | murine | Mm00435546_m1 |
| | factor receptor, beta | | |
| | polypeptide | | |
| lnsr | Insulin receptor | murine | Mm00439693_m1 |
| Prlr | Prolactin receptor | murine | Mm00599957_m1 |
| Nr3c1 (Gr) | Glucocorticoid receptor | murine | Mm00433832_m1 |
| | (nuclear receptor | | |
| | subfamily 3, | | |
| | group C, member 1) | | |
| Pdx1 | Pancreatic and duodenal | murine | Mm00435565_m1 |
| | homeobox 1 | | |
| FoxO1 | Forkhead box O1 | murine | Mm00490672_m1 |
| FoxO3 | Forkhead box O3 | murine | Mm01185722_m1 |
| Ccnb1 | Cyclin B1 | Murine | Mm00838401_g1 |
| Ccnd1 | Cyclin D1 | Murine | Mm00432359_m1 |
| Ccnd2 | Cyclin D2 | Murine | Mm00438071_m1 |
| Rb1 | Retinoblastoma 1, Rb1 | Murine | Mm00485586_m1 |
| E2f1 | E2F transcription factor 1 | Murine | Mm00432939_m1 |
| Ezh1 | Enhancer of zeste homolog 1 | Murine | Mm00468440_m1 |
| | (Drosophila) | | |
| Pdgfa | Platelet derived growth factor, | Murine | Mm01205760_m1 |
| | A polypeptide | | |
| Pdgfb | Platelet derived growth factor, | Murine | Mm00440677_m1 |
| | B polypeptide | | |
| Pdgfc | Platelet-derived growth factor, | Murine | Mm00480205_m1 |
| | C polypeptide | | |
| EZH1 | Enhancer of zeste | Human | Hs01016789_m1 |
| | homolog 1 (Drosophila) | | |

Histology, Immunofluorescence and Immunohistochemistry.

We performed standard histological paraformaldehyde fixation, paraffin-embedding and immunostaining protocol as described in Chen et al. (2009) Genes Dev. 23, 975-985. Briefly, immunohistochemical analysis was performed on 5-μm-thick sections of pancreatic tissues or islet sections after antigen retrieval (DAKO) and using the following primary antibodies: guinea pig anti-insulin (1:400, Sigma), mouse anti-glucagon (1:200, Sigma), rabbit anti-somatostatin (1:200, Dakocytomation), rabbit anti-pancreatic polypeptide (1:200, Dakocytomation), rabbit anti-Ezh2 (1:100, Epigentek), rabbit anti-PDGFR-α (1:50, Novus Biologicals), sheep anti-PDGFR-β (1:100, Sigma), rabbit anti-phospho-PDGFR-α (Tyr720) (1:50, Santa Cruz Biotechnologies), rabbit anti-phospho-p44/42 MAPK (Erk1/2) (Thr202/Tyr204) (1:50, Cell Signaling), rabbit anti-phospho-Rb (Ser780) (1:100, Cell Signaling), rabbit anti-cyclin D1 (1:50, Cell Signaling), rabbit anti-cyclin D2 (1:50, Cell Signaling), mouse anti-PDX1 (1:50), mouse monoclonal anti-Ki67 (1:100, Novocastra) and mouse monoclonal anti-BrdU (1:100, Sigma). We detected immune complexes with secondary antibodies conjugated with either Cy3, fluorescein isothiocyanate (Jackson ImmunoResearch) or horseradish peroxidase (Vector Laboratories). After staining, images were directly collected on an AxioM1 microscope equipped with a CCD digital camera (Carl Zeiss), or on a Leica SP2 inverted confocal microscope.

For measuring pancreatic β-cell mass or BrdU by morphometry, three to six mice for each group were analysed. At least five sections separated by more than 300 μm (pancreases) or 50 μm (cultured islets) were immunostained and assessed for each sample. Images were analysed with an ImagePro program by observers blinded to genotype, and pancreatic β-cell mass and percentage of BrdU+β-cells were calculated as described in Chen et al. Polycomb protein Ezh2 regulates pancreatic 6-cell Ink4a/Arf expression and regeneration in diabetes mellitus. Genes Dev. 23, 975-985 (2009).

Pancreatic Islet ChIP Analysis.

ChIPs were performed using a chromatin immunoprecipitation kit from Millipore. Briefly, freshly isolated mouse pancreatic islets or cultured human islets after the indicated treatments were pooled, and fixed with 2% formaldehyde for cross-linking for 20 min at room temperature (25° C.). After washing, islets were dissolved in SDS lysis buffer containing a proteinase inhibitor cocktail followed by sonication to shear the chromatin. Precleared chromatin from 150 to 300 islets was used for each ChIP sample with incubation of 1 to 5 μg of the appropriate antibodies at 4° C. overnight. The antibodies used in the ChIP assays included mouse anti-E2F1 (Millipore), rabbit anti-E2F4 (Santa Cruz Biotechnologies), rabbit or mouse polyclonal IgG (Millipore). After incubation, the immunoprecipitated chromatin DNA was harvested, cross-link reversed, and purified. After measurement of DNA concentration by PicoGreen DNA assay (Invitrogen), equivalent amounts of chromatin DNA from every sample was quantified by real-time qPCR in ABI Prism 7300 detection system (Applied Biosystems). The sequences of the PCR primers and probes are listed in Table 3 below.

TABLE 3
qPCR Primers used for ChIP analysis on mouse Ezh2 and Actb
loci and human EZH2 locus.

| Location | | Forward (5′-3′) | Probe (5′-3′) | Reverse (5′-3′) |
|---|---|---|---|---|
| Set 1 | Mouse | TTACCAACCCGA | CGTCTCCTCCCCT | GCGCTGAGTGGTT |
| | Ezh2 | GTTTTGAAC (SEQ | CCCCCTC (SEQ ID | CTCG (SEQ ID |
| | promoter | ID NO: 28) | NO: 29) | NO: 30) |
| Set 2 | Mouse | ATAAAAGCGATG | TTGCTGCGTTTGG | GACTCCACTGCCTT |
| | Ezh2 | GCGATTGG (SEQ | CGCT (SEQ ID | CGATGTC (SEQ ID |
| | exon 1 | ID NO: 31) | NO: 32) | NO: 33) |
| Set 3 | Mouse | AACGCCTTTTCA | TGGGTTTTTAATG | AAACTACATTCAAC |
| | Ezh2 | GTTTCAGG (SEQ | GCACATAAAACAT | TGTGACAGCA (SEQ |
| | intron 1 | ID NO: 34) | TTGA (SEQ ID | ID NO: 36) |
| | | | NO: 35) | |
| Actb | Mouse | CCGCCGTTCCGA | CCTTTTATGGCTC | GCCGCCGGGTTTT |
| | Actb | AAGTT (SEQ ID | GAGTGGCC (SEQ | ATAGG (SEQ ID |
| | promoter | NO: 37) | ID NO: 38) | NO: 39) |
| EZH2 | Human | GGCCCTGTGATT | CAATAAAAGCGAT | GGACCGAGCGCCA |
| | EZH2 | GGAC (SEQ ID | GGCGATTGGGCT | AAC (SEQ ID NO: 42) |
| | promoter | NO: 40) | GC (SEQ ID NO: 41) | |

Human Pancreas and Islet Studies.

Human pancreases or islets from organ donors were procured by arrangement with the National Disease Resource Interchange and the University of Alabama, Birmingham with appropriate consent. Institutional review board approval for research use of tissue was obtained from Stanford University School of Medicine. For pancreas studies, fresh human pancreases were processed in our laboratory into paraffin sections using standard histological protocols. Four pancreases from juvenile donors (1, 2, 3, 7 years old) and four pancreases from adult donors (22, 34, 55, 56 years old) were used for immunohistology studies here. Similar immunohistology methods and antibodies as those used for mouse pancreas tissues were used to detect PDGFR-α, phospho-ERK1/2 (pERK1/2), phospho-RB (Ser780) (pRB(Ser780), EZH2 and insulin on human pancreas sections. Experiments were repeated on multiple pancreas sections for every donor pancreas and similar results were obtained.

For islet studies, human islets were isolated either at Pittsburgh University Medical Center or the University of Alabama, Birmingham. Eight independent human islet preparations from juvenile donors at the age of 8 months, 1.0, 1.5, 2, 3, 4, 5 and 6 years, and three adult islet preparations from donors at the age of 39, 48 and 56 years were used in this study. After isolation, fresh islets were hand-picked and transferred to RPMI-1640 medium supplemented with 0.5% FBS, 0.2% BSA and 2% penicillin/streptomycin for an overnight recovery before experiments. Similar to the experiments performed with mouse islets, equilibrated human islets were exposed to recombinant human PDGF-AA, without or with addition of pharmacological inhibitors, including Sunitinib (Selleck) and U0126 (Sigma) for 2 days, followed by islet RNA purification for real-time RTPCR analyses of gene expression. To detect in vitro human islet β-cell proliferation stimulated by PDGF-AA, human islets were exposed to 50 μM BrdU for 24 h. Afterwards, human islets were processed in a similar way as in mouse islet studies for immunostaining of insulin, glucagon, PDX1 and BrdU. β-Cell proliferation rate was determined by quantifying percentage of insulin+ cells with BrdU+. More than 50 islets per group were counted for determining cultured human islet β-cell proliferation rate.

### Example 1

Age-Dependent β-Cell Pdgfr-α Loss.

During physiological growth in humans, mice and other species, juvenile β-cells expand by self-renewal (Dor et al. Adult pancreatic β-cells are formed by self-duplication rather than stem-cell differentiation. Nature 429, 41-46 (2004); Kushner et al. Cyclins D2 and D1 are essential for postnatal pancreatic β-cell growth. Mol. Cell. Biol. 25, 3752-3762 (2005)) while maintaining their hallmark functions. Pancreatic β-cell proliferation decreases rapidly in juvenile mice and humans, then declines more slowly in adults (Teta et al. Very slow turnover of β-cells in aged adult mice. Diabetes 54, 2557-2567 (2005); Meier et al. β-Cell replication is the primary mechanism subserving the postnatal expansion of β-cell mass in humans. Diabetes 57, 1584-1594 (2008)). Studies indicate that age-dependent increases of p16INK4a, a cyclin-dependent kinase inhibitor encoded by Cdkn2a, restrict proliferation of mouse and human β-cells and other tissues with ageing (Krishnamurthy, J. et al. p16INK4a induces an age-dependent decline in islet regenerative potential. Nature 443, 453-457 (2006); Zindy et al. Expression of the p16INK4a tumor suppressor versus other INK4 family members during mouse proliferation and aging. Oncogene 15, 203-211 (1997)). The polycomb group protein Ezh2 represses Cdkn2a and promotes β-cell proliferation in juvenile mouse islets (Chen et al. Polycomb protein Ezh2 regulates pancreatic β-cell Ink4a/Arf expression and regeneration in diabetes mellitus. Genes Dev. 23, 975-985 (2009)). Ezh2 is a histone methyltransferase crucial for trimethylation of histone H3 on Lys27 (H3K27me3), a modification mediating transcriptional repression (Cao et al. Role of histone H3 lysine 27 methylation in Polycomb-group silencing. Science 298, 1039-1043 (2002); van der Vlag & Otte, Transcriptional repression mediated by the human polycomb-group protein EED involves histone deacetylation. Nature Genet. 23, 474-478 (1999)). Ezh2 expression in mouse and human β-cells declines rapidly in juveniles, then slowly in adults (Chen et al. (2009), supra), similar to the tempo of age-dependent decline in β-cell proliferation (Teta et al. (2005), supra; Meier et al. (2008), supra). Conditional Ezh2 inactivation in β-cells accelerated loss of H3K27me3 repression at Cdkn2a, resulting in premature p16INK4a expression, impaired β-cell proliferation, reduced β-cell mass and inadequate β-cell regenerative recovery after β-cell ablation (Chen et al. (2009), supra).

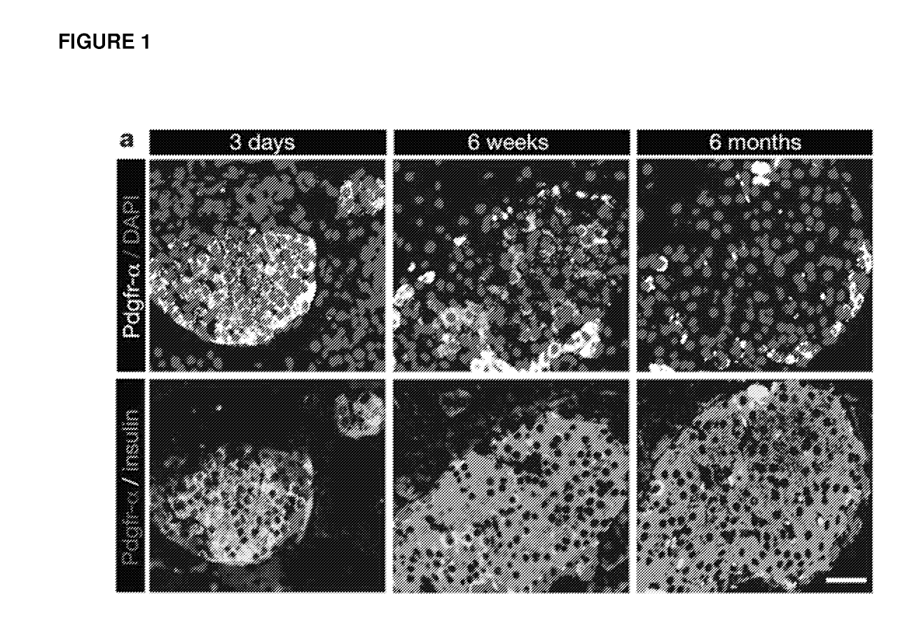

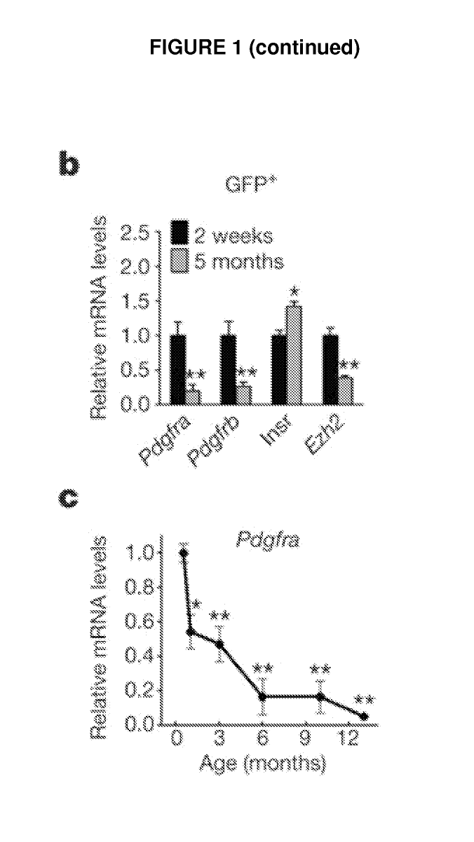

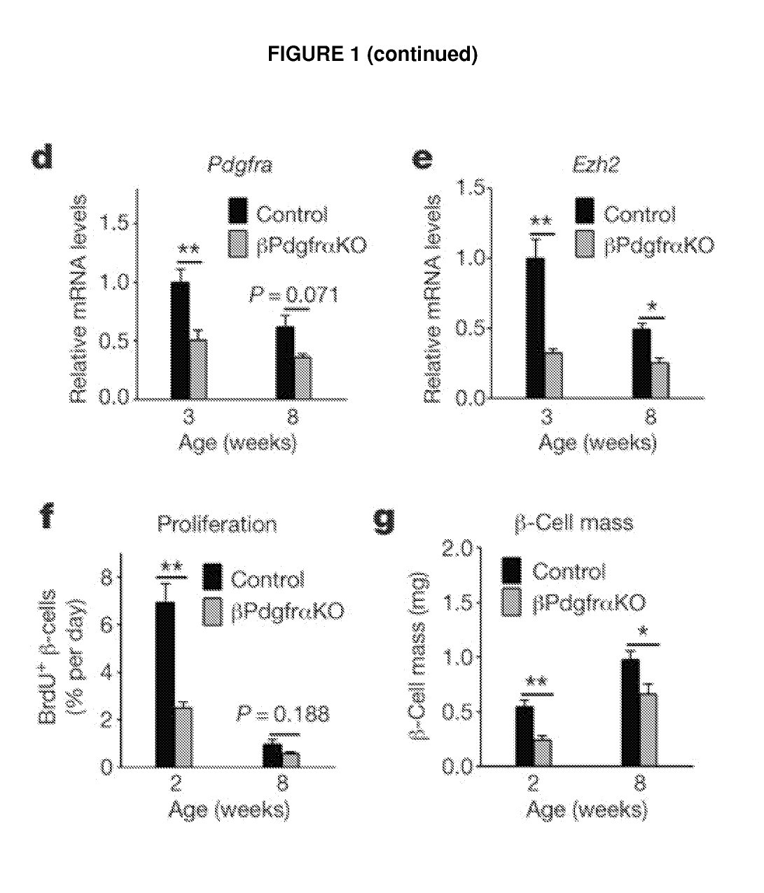

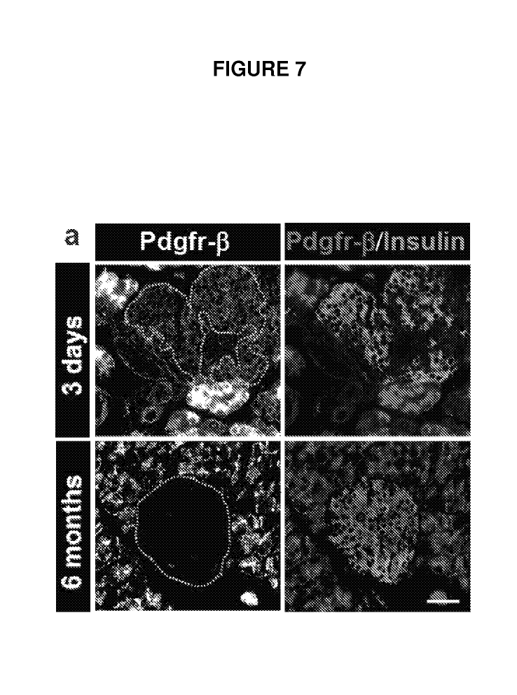

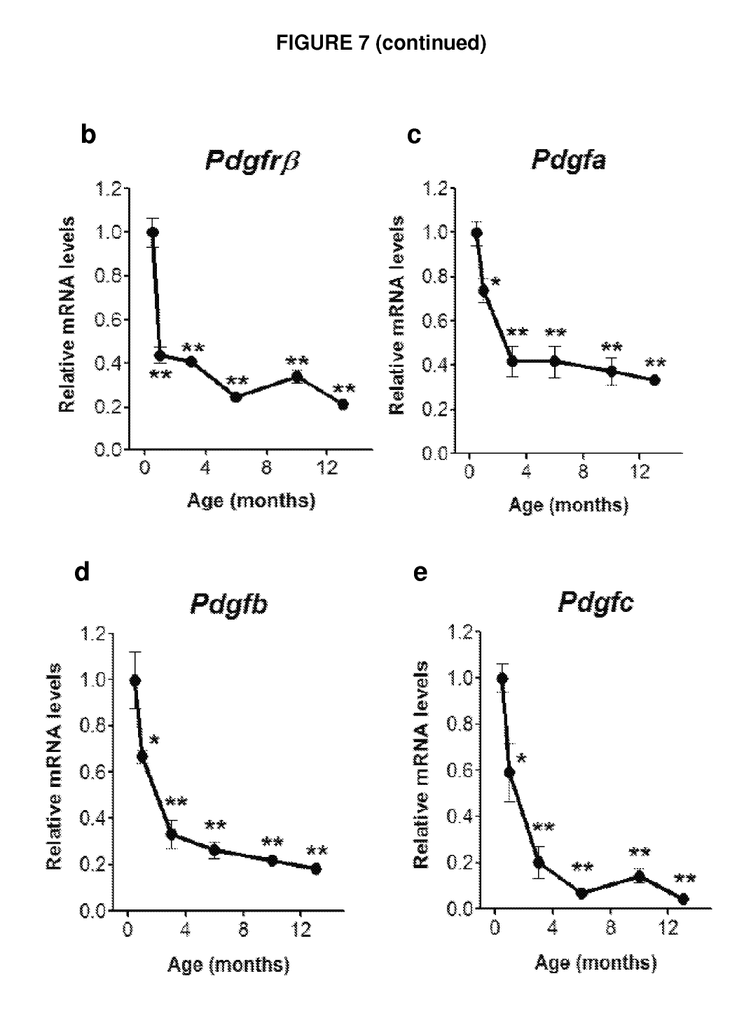

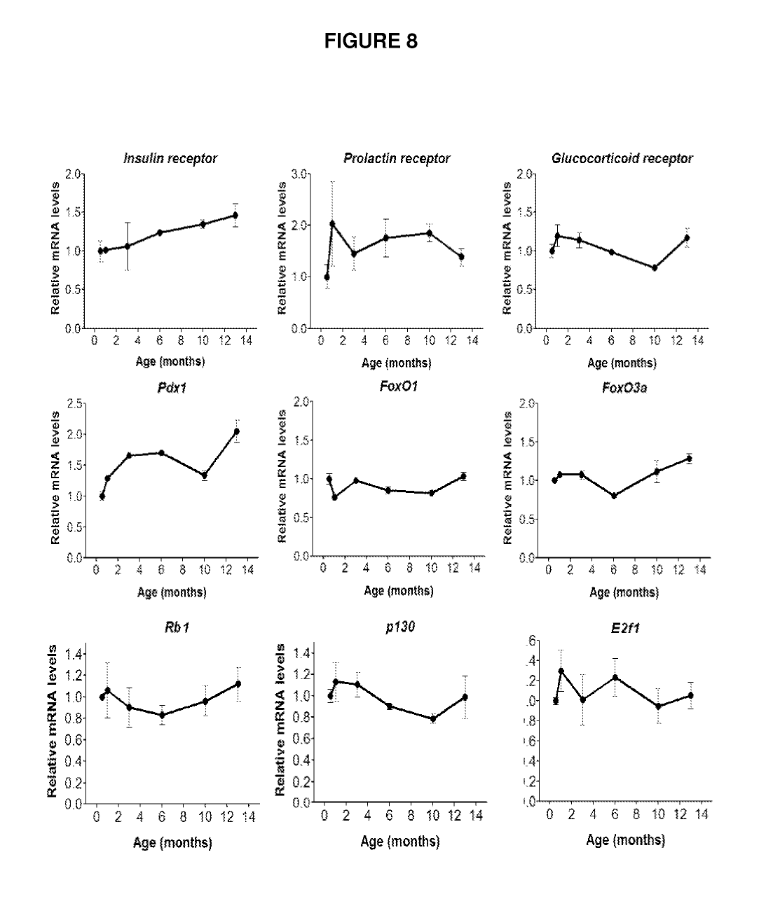

To investigate whether Pdgf signalling might regulate islet β-cell proliferation and Ezh2 expression, we assessed Pdgf receptor and ligand expression in islets and β-cells. Immunohistology revealed that Pdgfr-α and Pdgfr-β levels were markedly reduced in islet β-cells at 6 weeks and 6 months of age, compared to neonatal islets (FIG. 1a and FIG. 7a). Real-time polymerase chain reaction with reverse transcription (RTPCR) studies of β-cells purified by fluorescence-activated cell sorting (FACS) confirmed that Pdgfra, Pdgfrb and Ezh2 messenger RNA levels were reduced in adult β-cells at 5 months compared to 2-week-old β-cells (FIG. 1b). Further RTPCR analyses of isolated islets from wild-type mice ranging from 2 weeks to 13 months revealed an age-dependent decline of islet mRNAs encoding Pdgfra, Pdgfrb and Pdgf ligands (FIG. 1c and FIG. 7b-e). In contrast, islet mRNAs encoding the insulin receptor, prolactin receptor, glucocorticoid receptor and intrinsic regulators of β-cell proliferation like Pdx1, Foxo1, Foxo3, Rb1 (hereafter referred to as Rb), p130 (also known as Rb12) and E2f1, did not decline in mice with advancing age (FIG. 8). Thus, reduced β-cell levels of Pdgf receptors and ligands corresponded with Ezh2 loss and age-dependent reduction of β-cell replication.

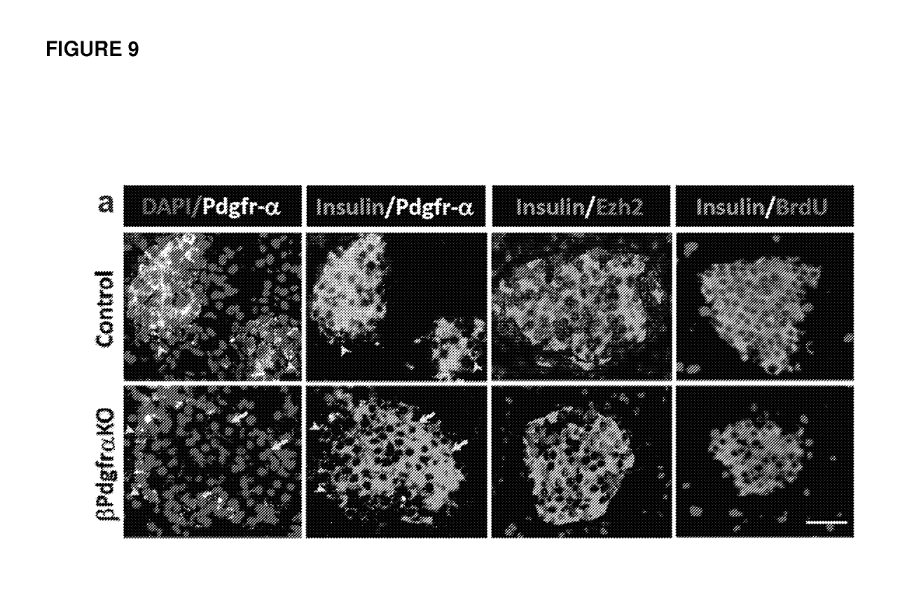

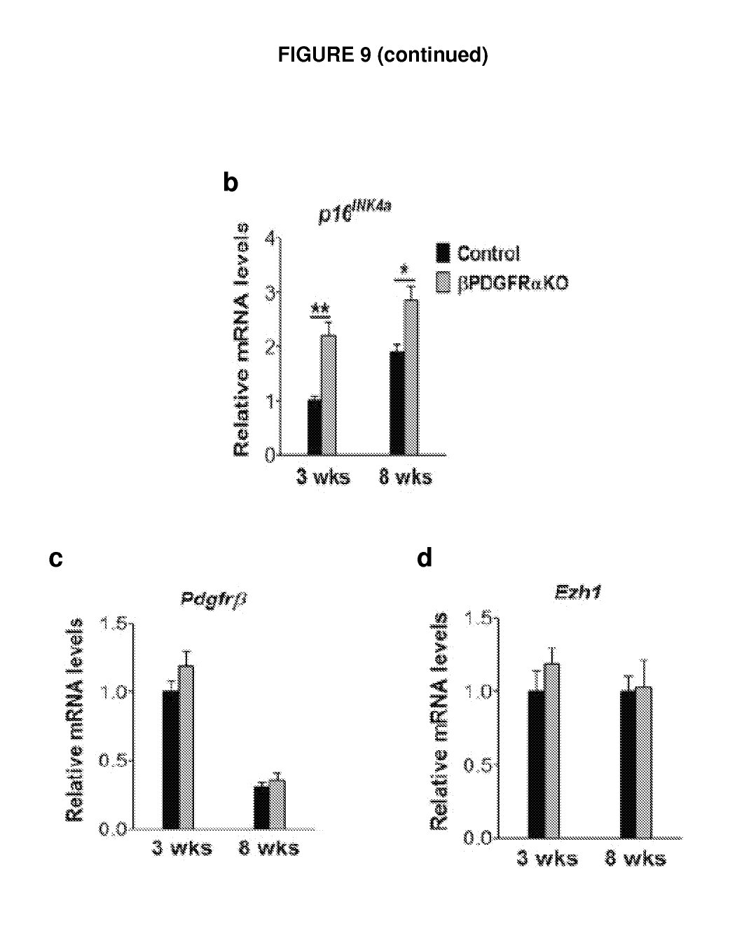

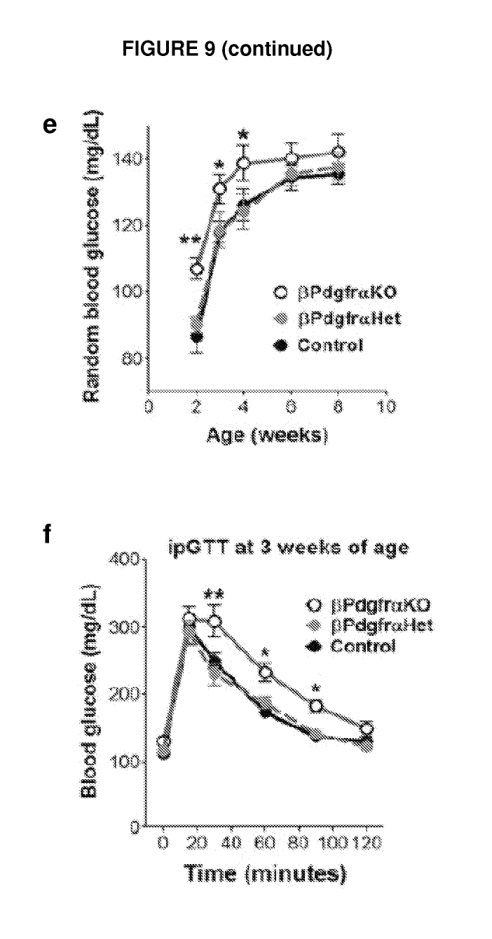

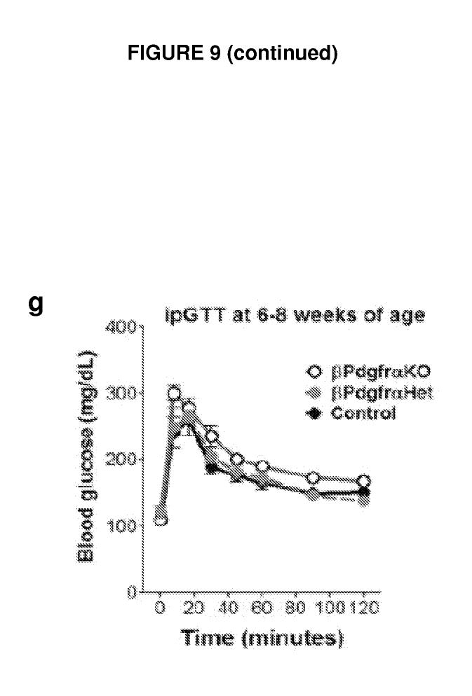

To assess whether premature Pdgfr loss might compromise Ezh2-dependent neonatal β-cell proliferation, we generated RIP-Cre; Pdgfra^{f/f} mice (abbreviated to βPdgfraKO) that permit conditional Pdgfra inactivation in islet β-cells and loss of detectable Pdgfra mRNA and protein expression (FIG. 1d and FIG. 9a). Compared to controls, β-cell Ezh2 protein and islet Ezh2 mRNA levels were reduced whereas p16INK4a mRNA levels were increased in βPdgfraKO islets (FIG. 1 e and FIG. 9a, b); in contrast, Pdgfrb and Ezh1 mRNA levels were unchanged in βPdgfraKO islets (FIG. 9c, d). In 2-3-week-old βPdgfraKO mice, we detected a threefold reduction in the β-cell proliferation rate and a 50% reduction in β-cell mass (FIG. 1f, g). βPdgfraKO mice at 3 weeks had mild hyperglycaemia during ad libitum feeding and impaired glucose tolerance in a glucose challenge test (FIG. 9e, f). Thus, premature loss of Pdgfra impairs β-cell proliferation and expansion and glucose control in young mice.

### Example 2

### Pdgfr-α Controls Adult β-Cell Regeneration

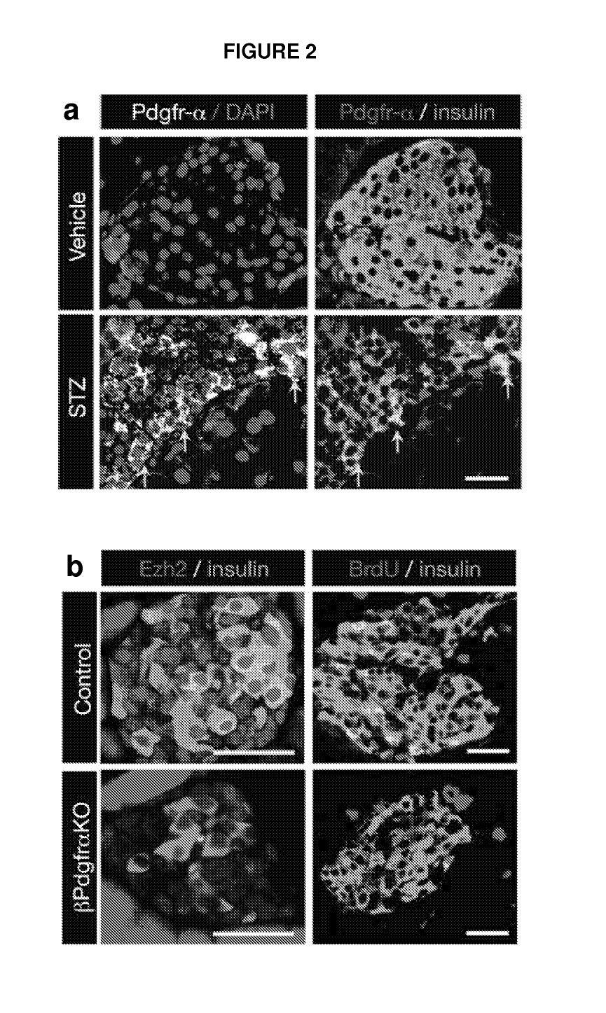

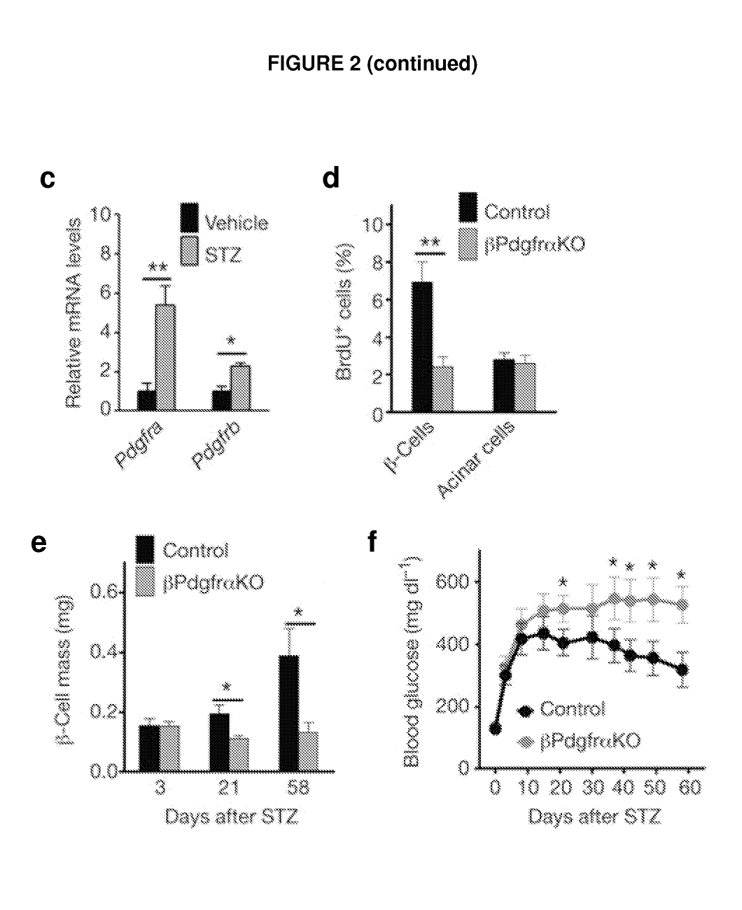

Destruction of β-cells by streptozotocin (STZ) in mice provokes 6-cell proliferation with regeneration of 6-cell mass (Krishnamurthy, J. et al. p16INK4a induces an age-dependent decline in islet regenerative potential. Nature 443, 453-457 (2006); Chen (2009) supra). Following STZ exposure, Pdgfra and Pdgfrb were induced in wild-type islets (FIG. 2a, c), suggesting possible roles for Pdgfr signalling in adult β-cell regeneration. Consistent with this view, β-cell mass in control mice gradually recovered after STZ treatment, accompanied by increased β-cell Ezh2 expression and proliferation; in contrast, βPdgfraKO β-cells failed to increase Ezh2 or proliferation to restore β-cell mass (FIG. 2b, d, e). βPdgfrαKO mice developed severe diabetes that persisted throughout the course of our studies (FIG. 2f). In contrast, control mice became moderately diabetic immediately after STZ challenge, but achieved restored glucose control 3-4 weeks thereafter (FIG. 2f), coinciding with increases of β-cell mass (FIG. 2e). Thus, Pdgfr-α is also required in adult β-cells for compensatory proliferation in experimental diabetes.

### Example 3

### PDGFR-α Promotes Functional β-Cell Expansion

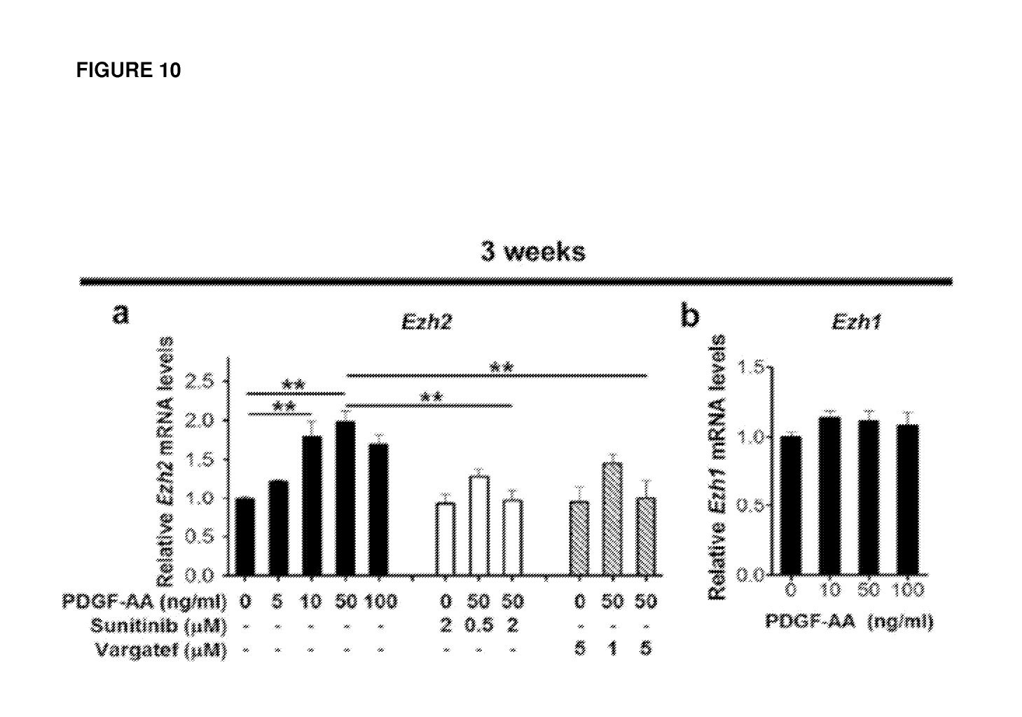

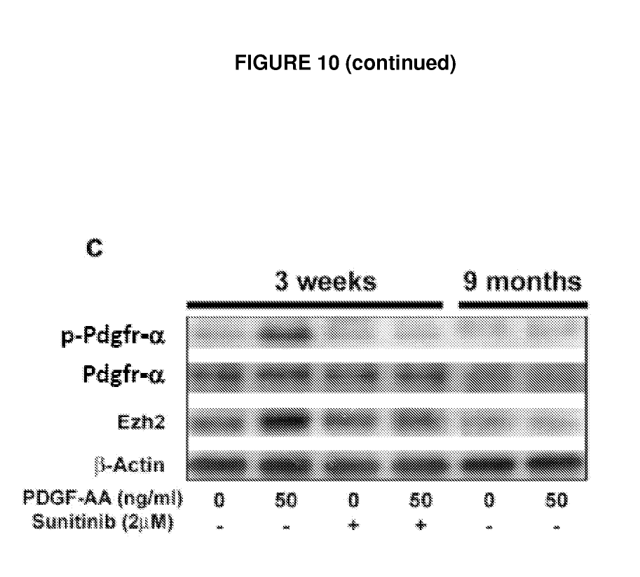

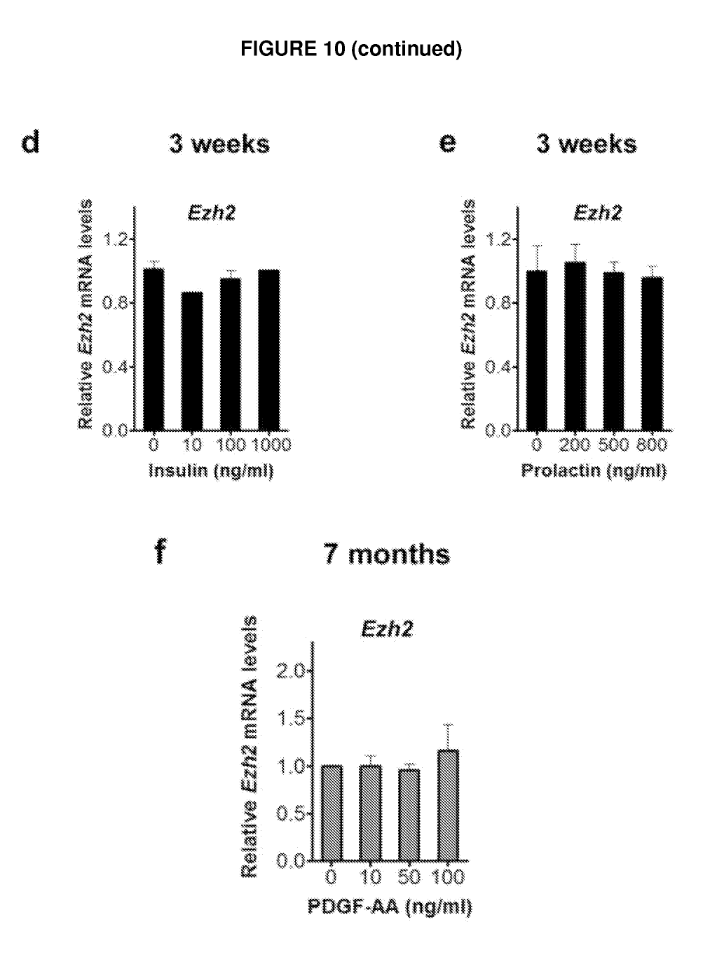

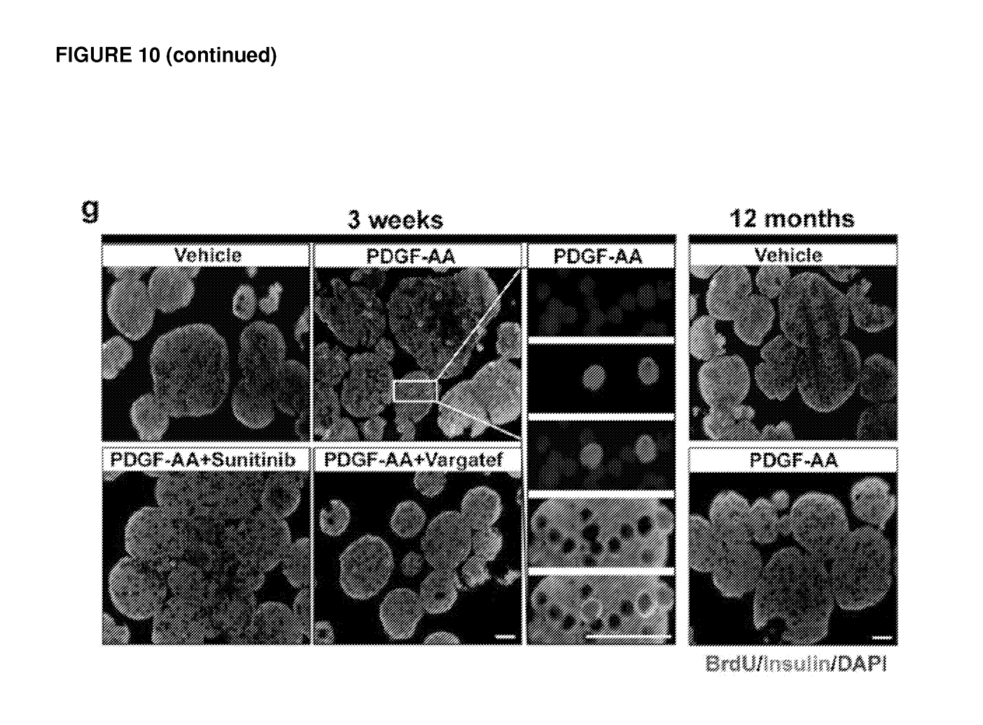

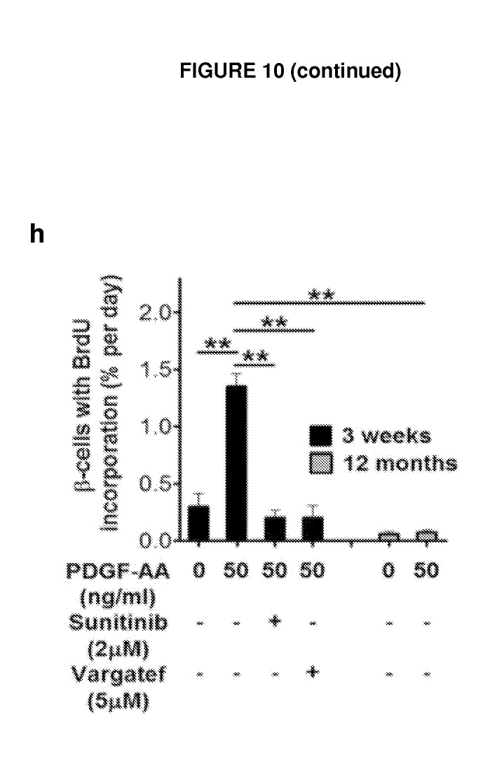

To test whether enhanced Pdgfr signalling might promote β-cell expansion, we exposed islets from juvenile and adult mice to physiological concentrations of platelet-derived growth factor-AA (PDGF-AA). Exposure of 3-week-old islets to PDGF-AA increased phospho-Pdgfr-α levels and Ezh2 mRNA and protein levels, but not Ezh1 expression (FIG. 10a-c). In contrast, juvenile islets exposed to mitogens like insulin or prolactin did not detectably alter Ezh2 mRNA levels (FIG. 10d, e). Unlike in 3-week-old islets, levels of Ezh2 mRNA and protein were not significantly increased in 7- or 9-month-old adult islets exposed to PDGF-AA (FIG. 10c, f). PDGF-AA exposure increased β-cell proliferation in juvenile islets, as assessed by BrdU incorporation, but not in adult islets (FIG. 10g, h). Increased Ezh2 expression and β-cell BrdU incorporation were eliminated by simultaneous treatment with the receptor tyrosine kinase inhibitors Sunitinib20 or Vargatef21 (FIG. 10a, c, g, h). Thus, PDGF-AA exposure was sufficient to stimulate β-cell replication in juvenile islets, but loss of competence for Pdgf prevented this response to PDGF-AA in adult islets.

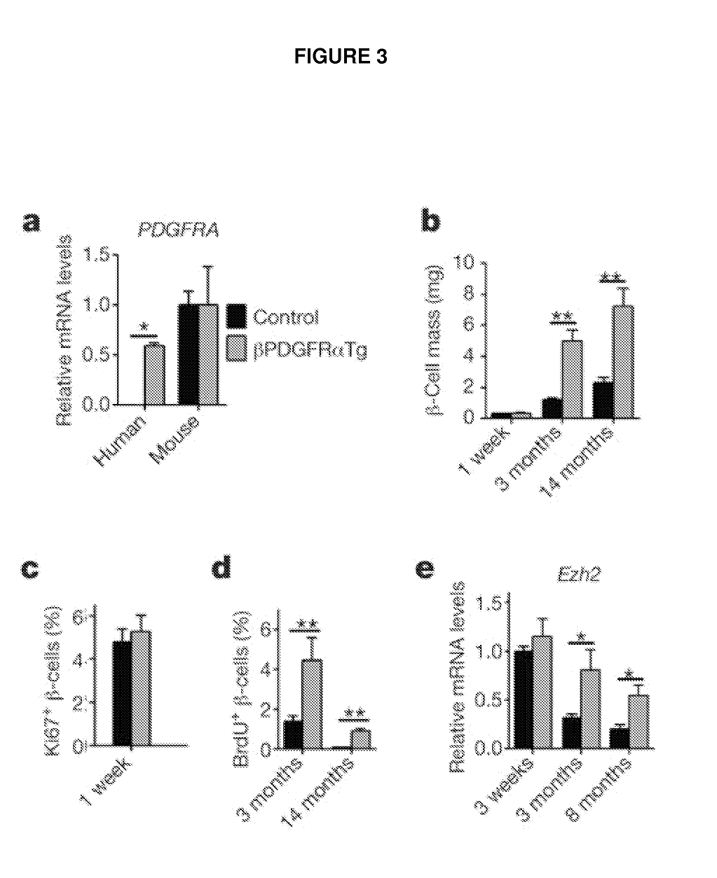

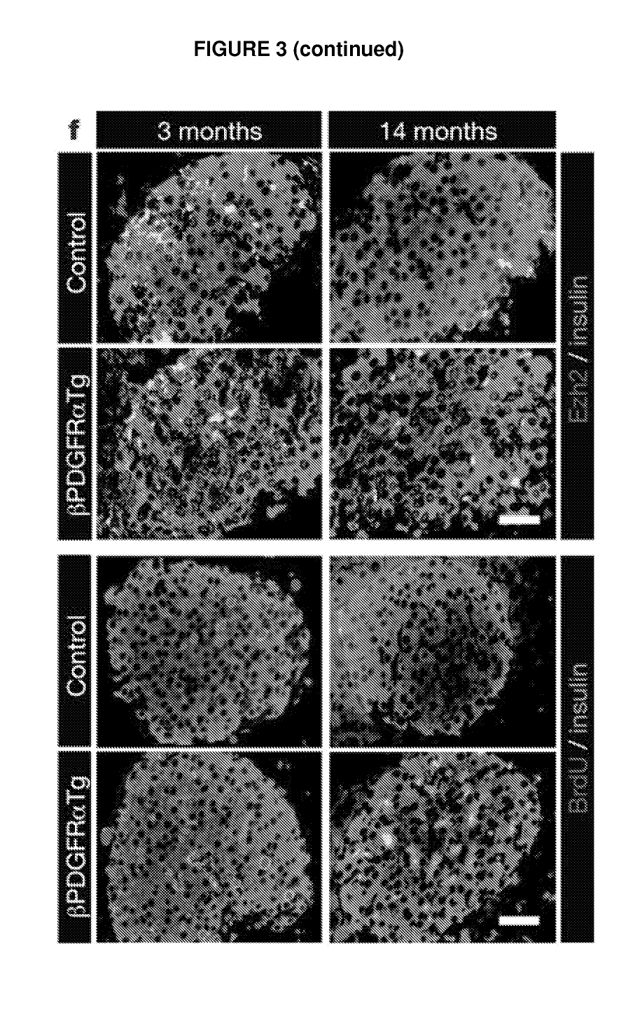

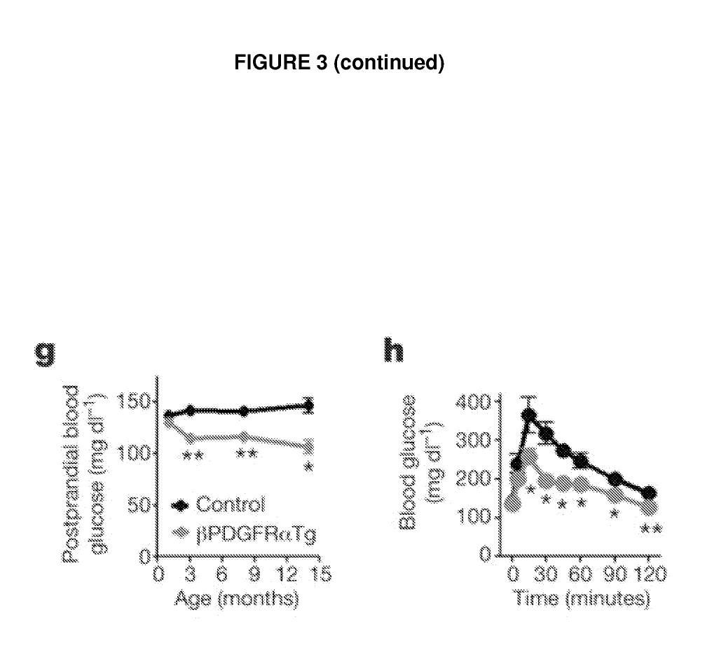

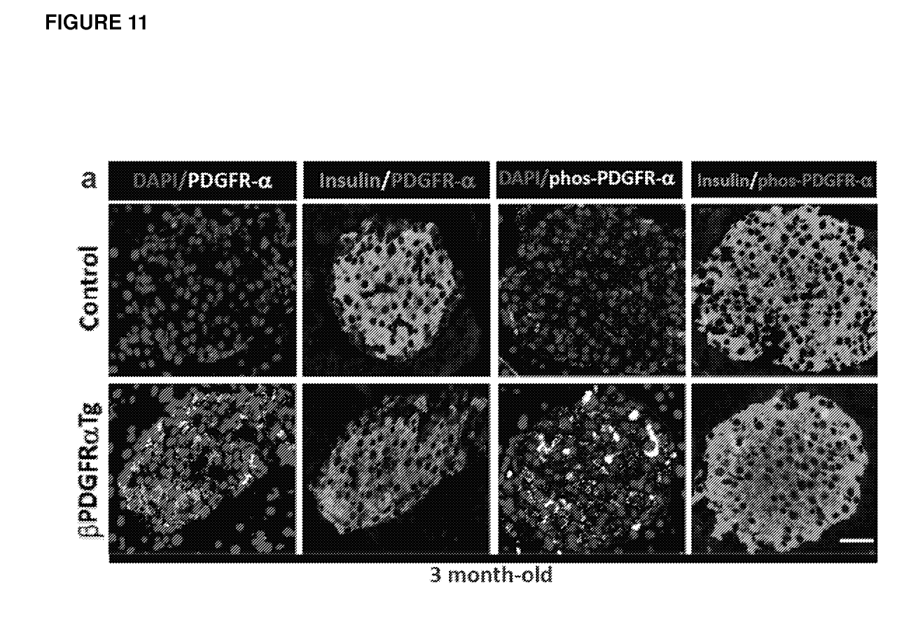

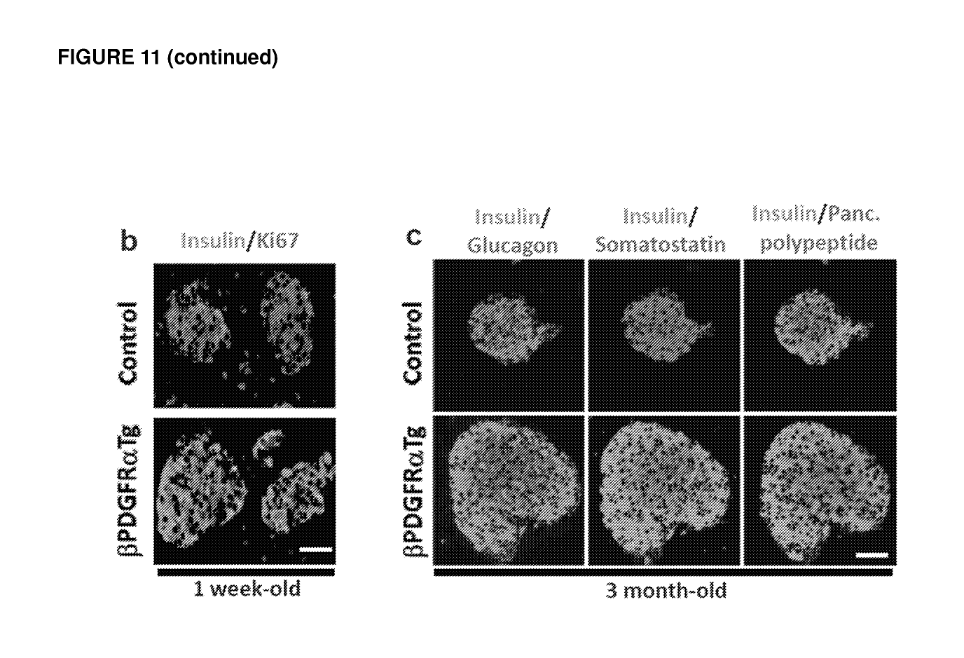

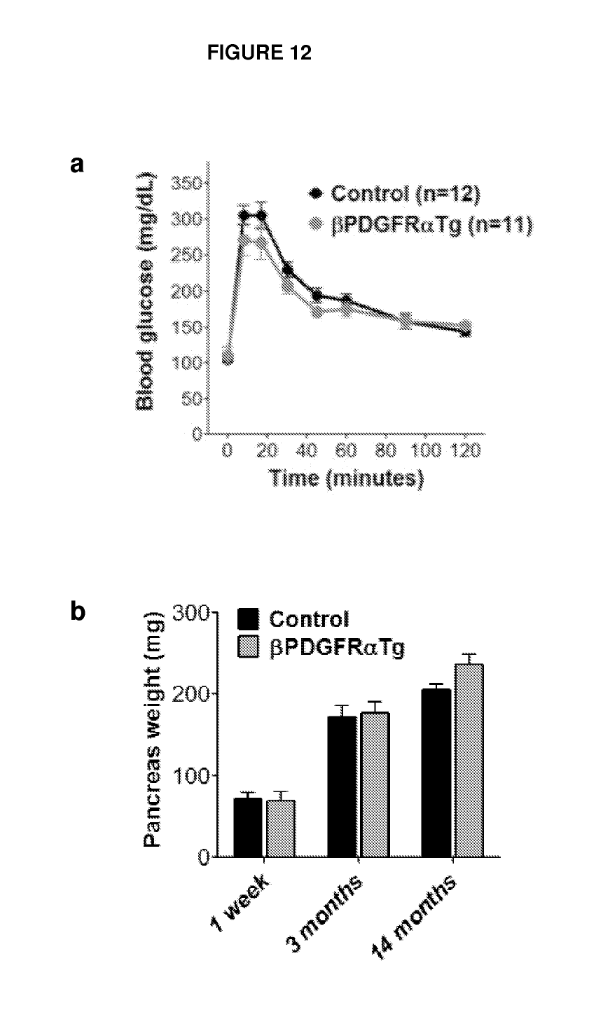

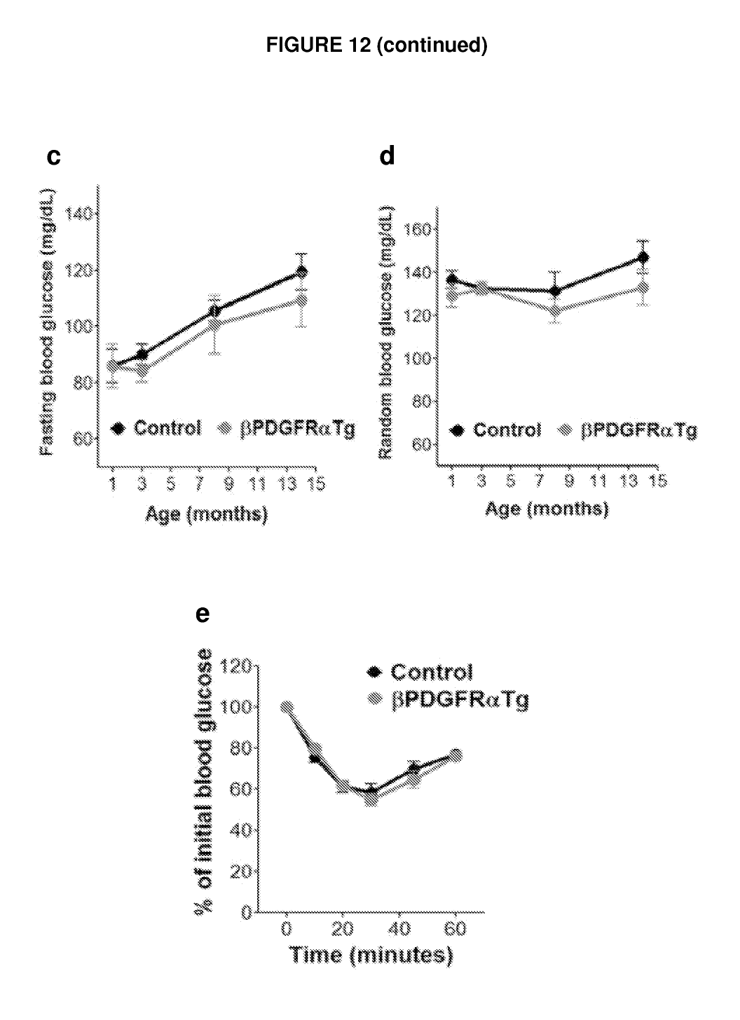

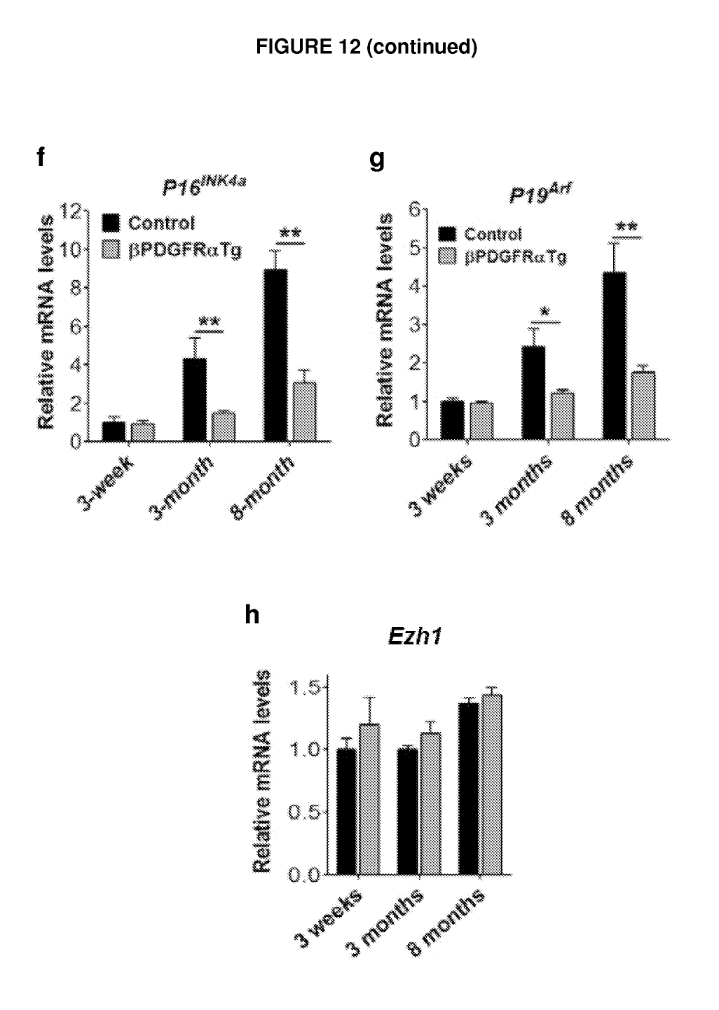

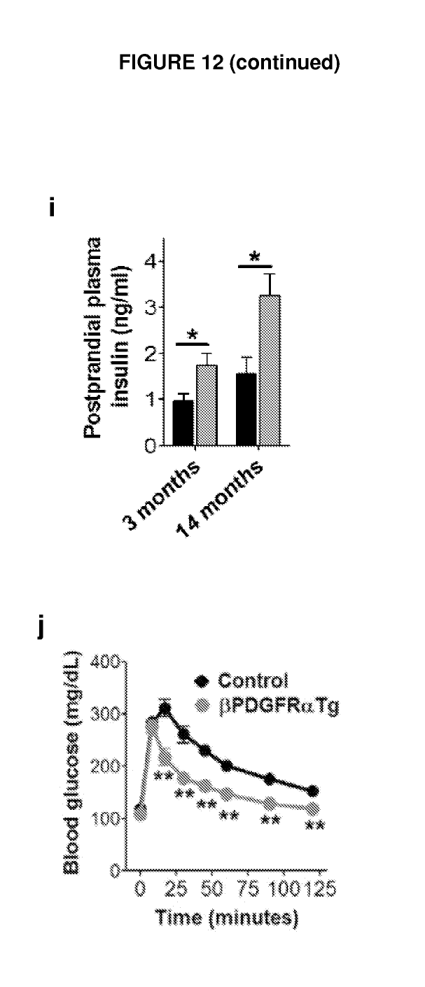

To investigate whether sustained PDGFR signalling permitted adult β-cell expansion in vivo, we intercrossed the RIP-Cre strain with mice harbouring a human PDGFRA^{D842V} allele encoding a ligand-independent activated receptor inserted at the ROSA26 (R26) locus (Moenning, A. et al. Sustained platelet-derived growth factor receptor α signaling in osteoblasts results in craniosynostosis by overactivating the phospholipase C-γ pathway. Mol. Cell. Biol. 29, 881-891 (2009)). A loxP-flanked transcriptional stop sequence permits Cre-dependent PDGFRA^{D842V} expression and activation of PDGF signalling in vivo (Moenning et al. (2009) supra). In contrast to littermate controls, mice with the RIP-Cre; R26-PDGFRA^{D842V} genotype (abbreviated to βPDGFRαTg) expressed human PDGFRA mRNA and PDGFR-α(D842V) protein in islet β-cells (FIG. 3a and FIG. 11a). Expression of R26-PDGFRA^{D842V} did not affect endogenous islet Pdgfra mRNA levels (FIG. 3a) or β-cell expansion and glucose control in neonatal or juvenile βPDGFRαTg mice (FIG. 3b, c and FIGS. 11b and 12a-d). By 3 months, β-cell Ezh2 expression had declined in control mice, but β-cell Ezh2 expression in βPDGFRαTg mice was maintained at elevated levels, a difference that persisted for 8-14 months (FIG. 3e, f). mRNA levels of islet p16INK4a and p19Arf, both targets of Ezh2, were reduced in βPDGFRαTg mice compared to those in littermate controls (FIG. 12f, g). In contrast, islet mRNA levels of Ezh1 in βPDGFRαTg and control mice were indistinguishable (FIG. 12h). β-Cell proliferation in 14-month-old βPDGFRαTg mice was increased ninefold compared to age-matched controls, a level of proliferation seen in 3-month-old control β-cells (FIG. 3d, f). β-Cell mass was increased in βPDGFRαTg mice at 3 months, and remained increased at 14 months compared to age-matched controls (FIG. 3b). In contrast, total pancreatic mass and islet architecture in βPDGFRαTg mice and controls were indistinguishable (FIGS. 11c and 12b). Thus, Pdgf signalling activation is sufficient to sustain adult β-cell expansion in vivo.

Prior studies indicate that β-cell expansion stimulated by mitogens can be complicated by disrupted β-cell function and impaired metabolic control (Vasavada et al. Targeted expression of placental lactogen in the β-cells of transgenic mice results in β-cell proliferation, islet mass augmentation, and hypoglycemia. J. Biol. Chem. 275, 15399-15406 (2000); Garcia-Ocana et al. Hepatocyte growth factor overexpression in the islet of transgenic mice increases β-cell proliferation, enhances islet mass, and induces mild hypoglycemia. J. Biol. Chem. 275, 1226-1232 (2000)). To assess the physiological impact of prolonged adult β-cell proliferation in βPDGFRαTg mice, systemic blood glucose and insulin levels were measured. Blood glucose levels during fasting and ad libitum feeding and responses to insulin challenge were indistinguishable in βPDGFRαTg mice and littermate controls (FIG. 12c-e). During re-feeding after overnight fast, βPDGFRαTg mice had a 25% reduction of blood glucose (FIG. 3g), associated with a modest elevation of plasma insulin levels (FIG. 12i), and both changes were sustained for up to 14 months. After overnight fast, glucose clearance was enhanced in glucose-challenged 3- or 14-month-old βPDGFRαTg mice (FIG. 3h and FIG. 12j). Thus, PDGFRαTg mice maintained normal glucose-regulated insulin release and glycaemic control, suggesting that β-cell function remained regulated in these mice.

### Example 4

PDGF-Induced β-Cell Expansion Requires Ezh2.

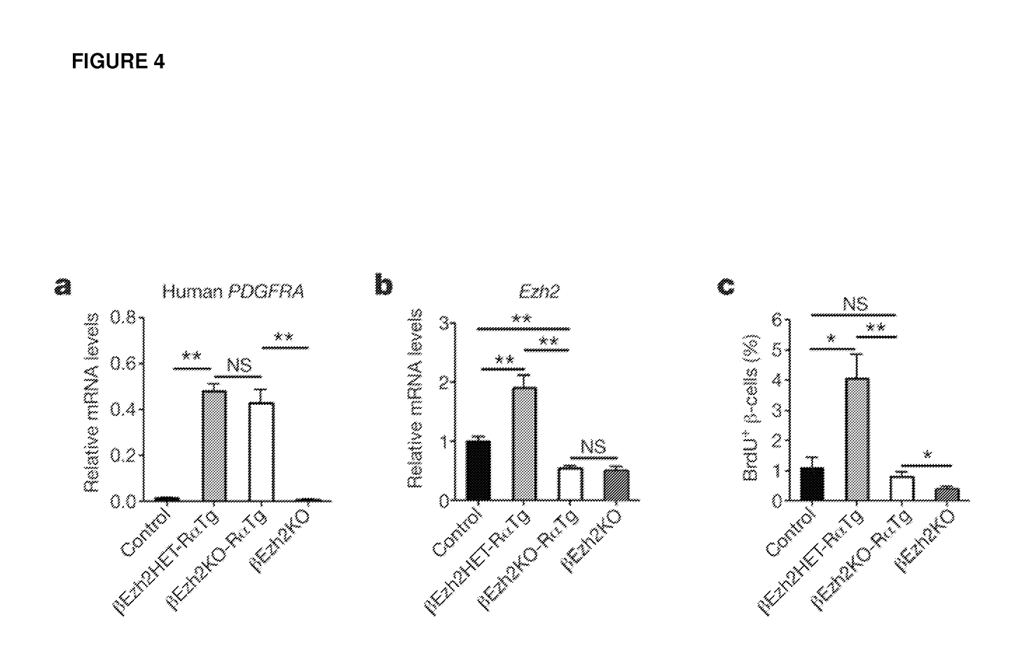

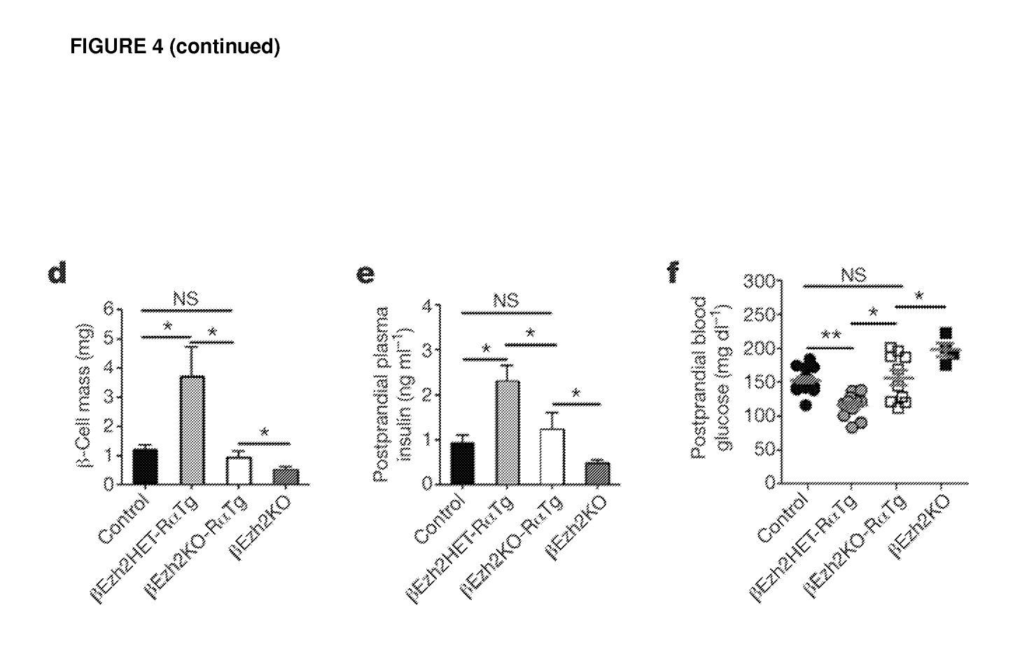

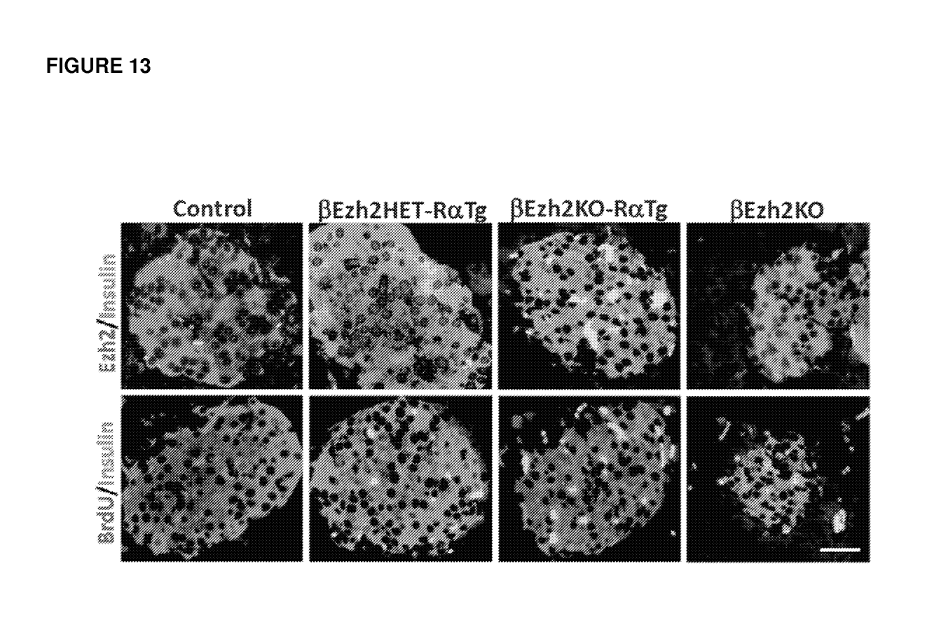

To test whether PDGFR-α-induced β-cell proliferation and expansion required Ezh2, mice permitting conditional inactivation of Ezh2 in βPDGFRαTg β-cells were generated. Intercrosses generated RIP-Cre; Ezh2^{f/f}; R26-PDGFRα^{D842V} mice (abbreviated to βEzh2KO-RαTg) and littermate or sibling RIP-Cre; Ezh2^{f/+}; R26-PDGFRα^{D842V} mice (abbreviated to βEzh2HET-RαTg) and RIP-Cre; Ezh2^{f/f} mice (abbreviated to βEzh2KO). Islet PDGFRA^{D842V} mRNA levels in both βEzh2KO-RαTg and βEzh2HET-RαTg mice were elevated and indistinguishable (FIG. 4a). By contrast, increased islet Ezh2 mRNA levels and β-cell Ezh2 protein were not observed in βEzh2KO-RαTg or βEzh2KO littermates (FIG. 4b and FIG. 13), confirming Cre-mediated deletion of Ezh2 in these mice. As expected, β-cell BrdU incorporation and mass were increased in βEzh2HET-RαTg mice (FIG. 4c, d and FIG. 13) compared to controls (either Ezh2^{f/f}, R26-PDGFRα^{D842V} or Ezh2^{f/+}, R26-PDGFRα^{D842V} genotype). However, neither β-cell BrdU incorporation nor mass were increased in βEzh2KO-RαTg mice compared to controls (FIG. 4c, d and FIG. 13). Consistent with these findings, we detected modest postprandial blood glucose reduction and increased blood insulin levels in βEzh2HET-RαTg mice, but not in βEzh2KO-RαTg mice (FIG. 4e, f). Thus, Ezh2 is required for β-cell expansion and metabolic changes from PDGFR-α activation in β-cells.

### Example 5

β-Cell PDGFR Controls Ezh2 Via Erk and Rb/E2f.

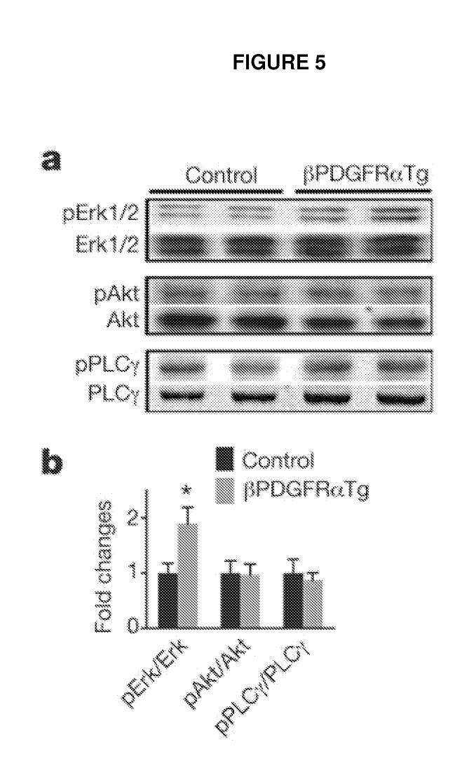

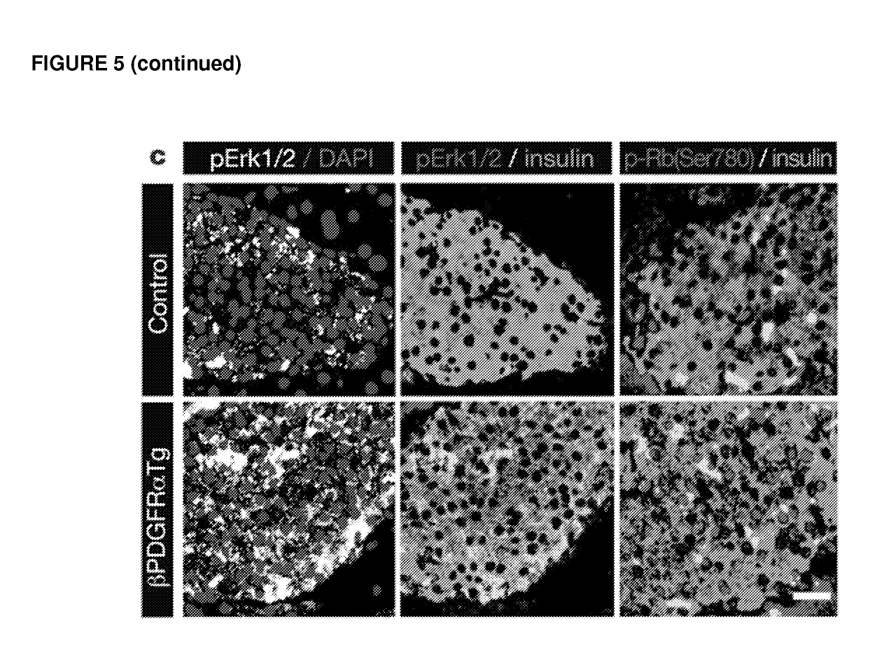

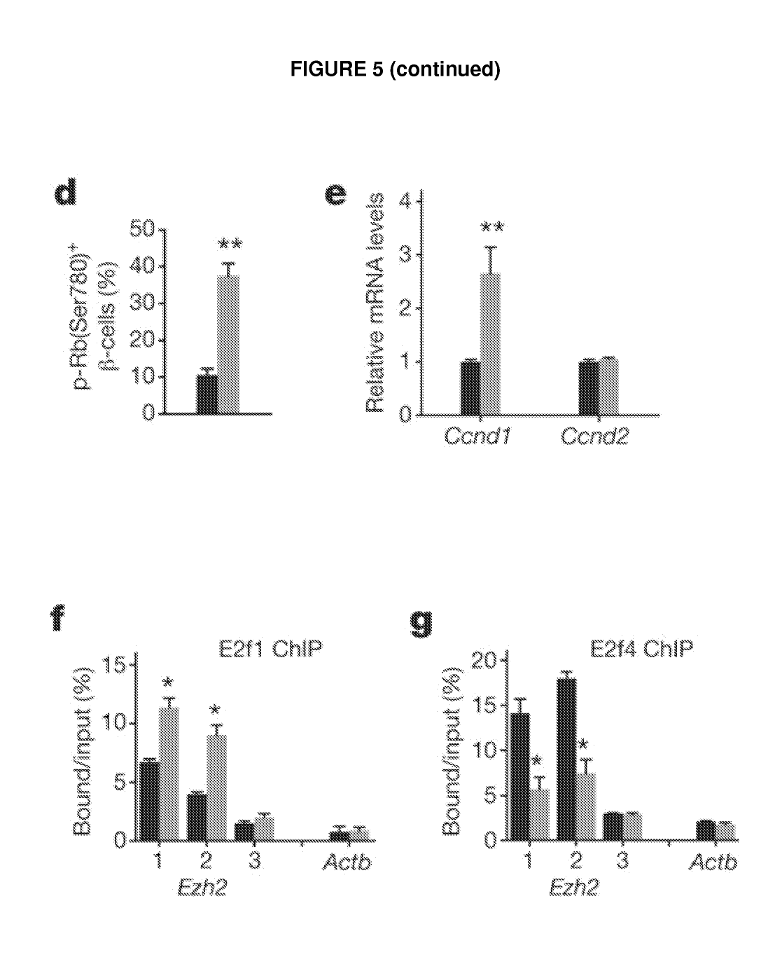

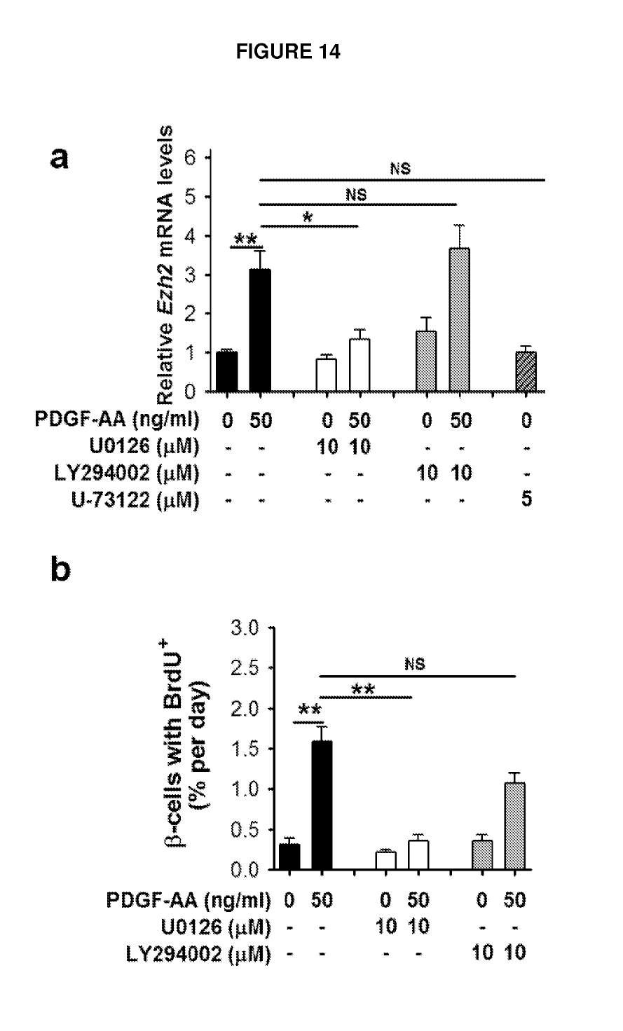

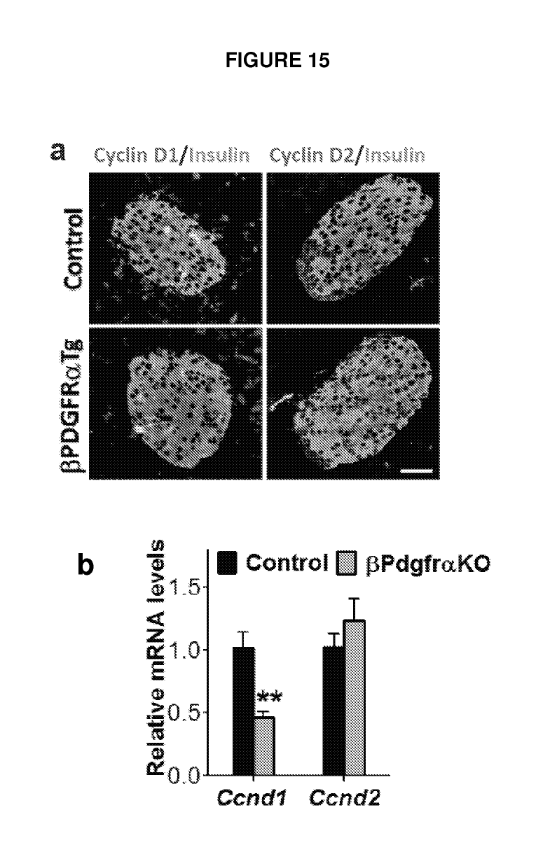

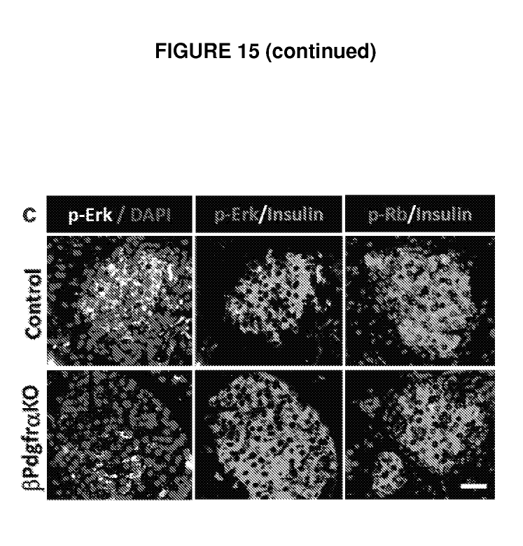

To elucidate the signalling basis of altered β-cell proliferation in βPDGFRαTg, βPdgfrαKO and ageing wild-type mice, we investigated Pdgf signal transduction factors. Pdgf receptors activate signalling elements (Schlessinger, J. Cell signaling by receptor tyrosine kinases. Cell 103, 211-225 (2000)), including the mitogen-activated protein kinase/extracellular signal-regulated kinase (Mapk/Erk), phosphatidylinositol 3-kinase (PI3K)/Akt and phospholipase PLC-γ. Ezh2 induction in juvenile wild-type islets exposed to PDGF-AA was blocked by U0126, which inhibits Erk1/2 activation (Duncia, J. V. et al. MEK inhibitors: the chemistry and biological activity of U0126, its analogs, and cyclization products. Bioorg. Med. Chem. Lett. 8, 2839-2844 (1998)), but not by LY294002 or U-73122, inhibitors of PI3K or PLC-γ signalling (FIG. 14a). Similarly, increased β-cell BrdU incorporation in islets exposed to PDGF-AA was blocked by U0126, but not by LY294002 (FIG. 14b), indicating that Erk mediates β-cell Pdgf signalling responses. We also detected increased phosphorylation of Erk1/2, but not phosphorylation of Akt or PLC-γ, in βPDGFRαTg islets compared to control islets (FIG. 5a, b). Immunohistology confirmed increased phospho-Erk1/2 levels in β-cells of βPDGFRαTg islets compared to controls (FIG. 5c). PDGF/ERK signalling activates cell cycle regulators to stimulate proliferation (Uhrbom, L., et al., Dissecting tumor maintenance requirements using bioluminescence imaging of cell proliferation in a mouse glioma model. Nature Med. 10, 1257-1260 (2004); Furstoss, O., et al., Cyclin E and cyclin A are likely targets of Src for PDGF-induced DNA synthesis in fibroblasts. FEBS Lett. 526, 82-86 (2002)), and βPDGFRαTg islets had elevated levels of Ccnd1 mRNA encoding cyclin D1, and increased β-cells with nuclear cyclin D1 and phosphorylated Rb protein (FIG. 5c-e and FIG. 15a). In contrast, loss of β-cell Pdgf signalling in βPdgfraKO mice diminished islet levels of Ccnd1 mRNA, and reduced numbers of phospho-Erk1/2+ and phospho-Rb+ β-cells (FIG. 15b, c).

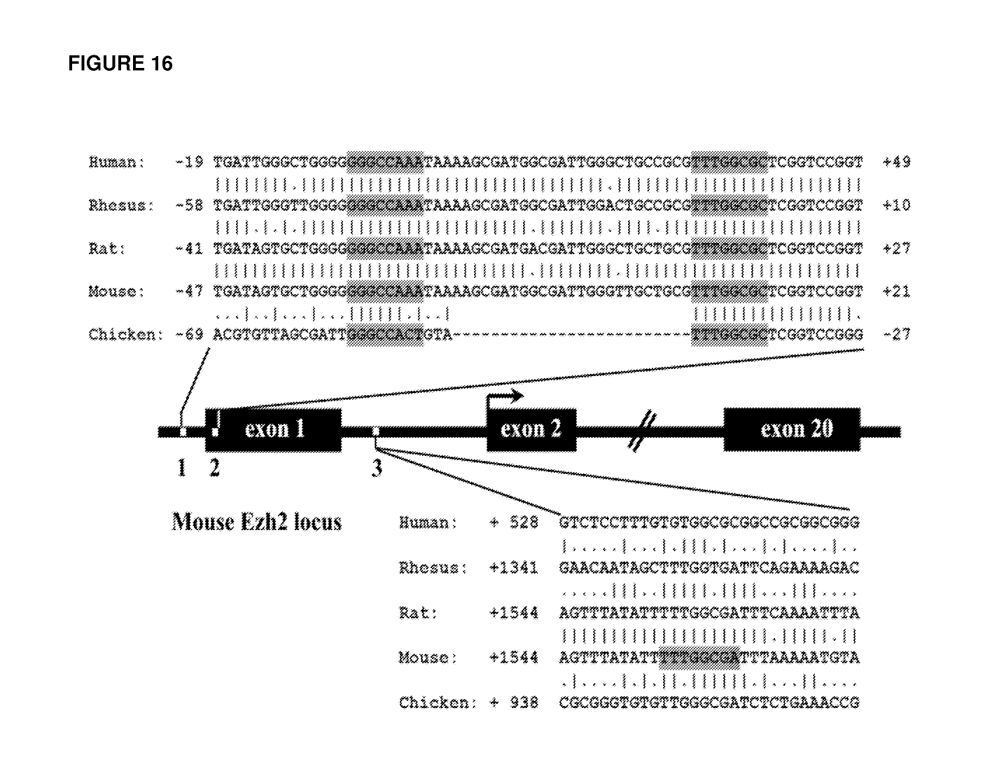

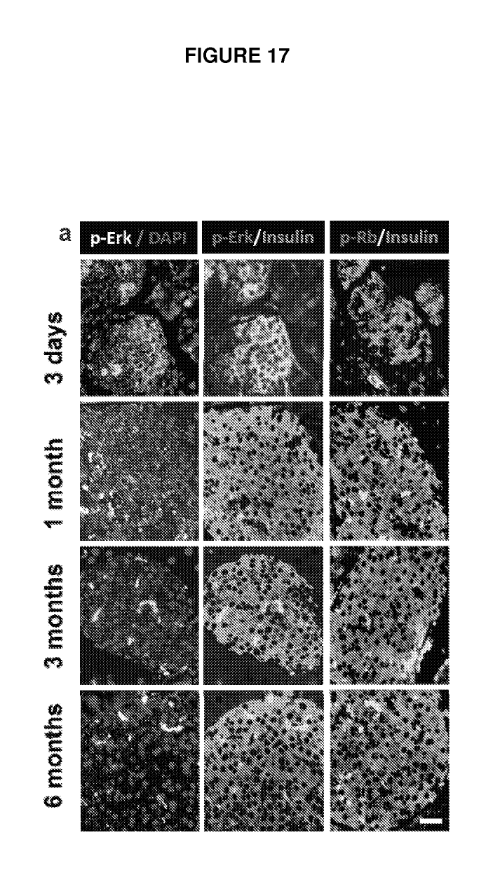

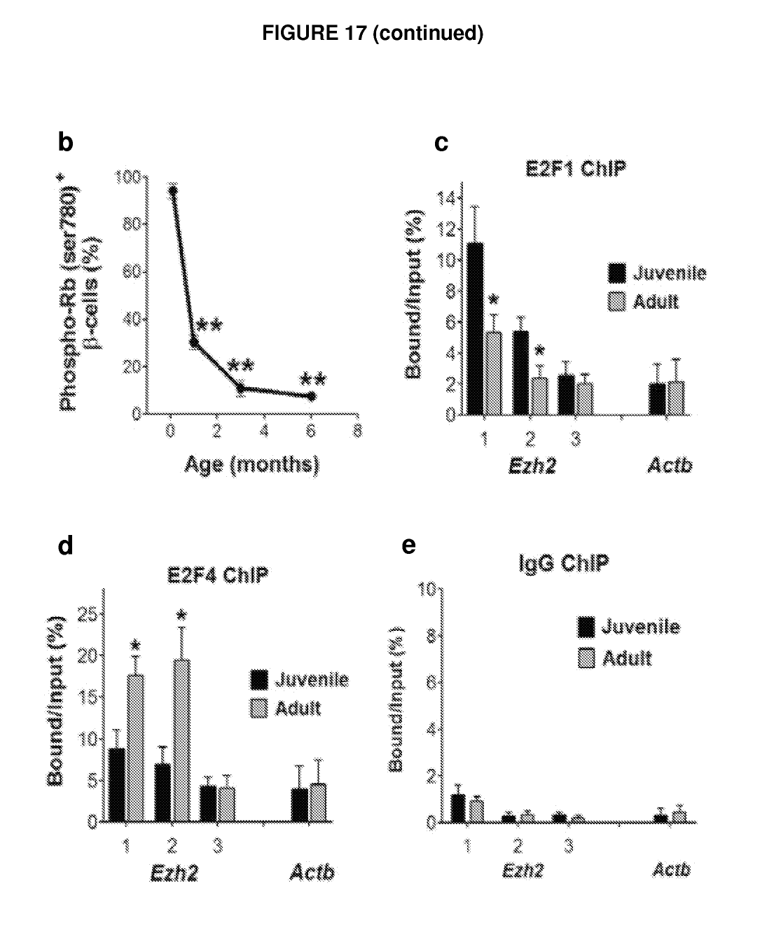

Rb protein phosphorylation regulates association of the E2f1 transcriptional activator and E2f4 transcription repressor with their targets (Bracken, A. P. et al. EZH2 is downstream of the pRB-E2F pathway, essential for proliferation and amplified in cancer. EMBO J. 22, 5323-5335 (2003); Moberg, K., Starz, M. A. & Lees, J. A. E2F4 switches from p130 to p107 and pRB in response to cell cycle reentry. Mol. Cell. Biol. 16, 1436-1449 (1996)). We identified two conserved candidate E2f-binding elements near the Ezh2 promoter (FIG. 16), and chromatin immunoprecipitation (ChIP) studies showed increased E2f1 association and reduced E2f4 association with these Ezh2 elements in βPDGFRαTg islets (FIG. 5f, g). In wild-type mice, levels of phospho-Erk1/2 and phospho-Rb declined with age in islet β-cells at a rate similar to declining Ezh2 and Pdgfra expression (FIG. 17a, b). ChIP studies also revealed that association of E2f1 with Ezh2 decreased, whereas association of E2f4 with Ezh2 elements increased in islets with age (FIG. 17c, d). Thus, age-dependent attenuation of β-cell Pdgfr-α altered Erk and Rb/E2f signalling, resulting in reduced Ezh2 expression.

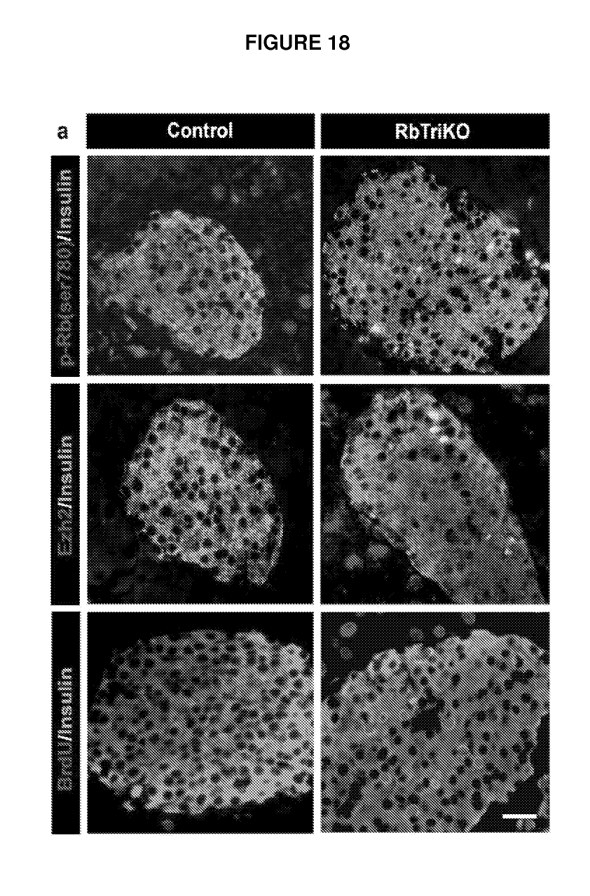

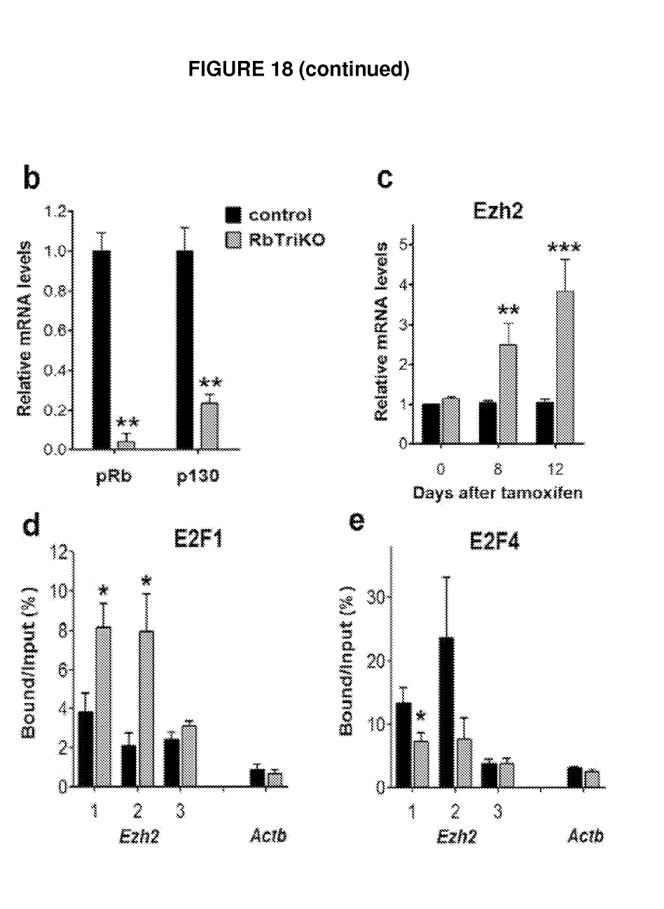

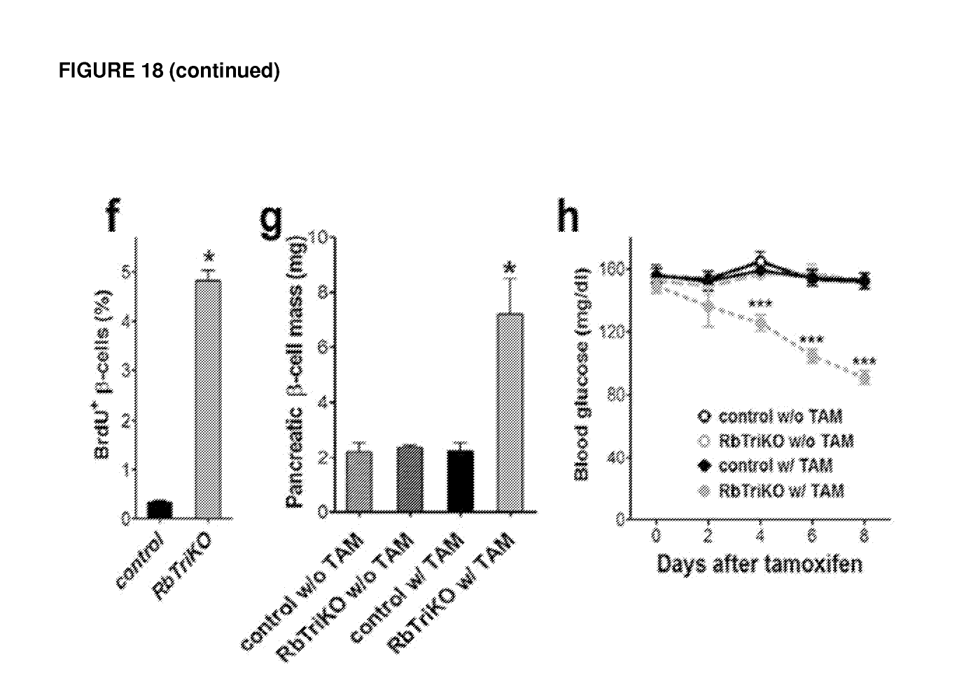

To verify that Rb/E2f signalling regulates β-cell Ezh2, we examined triple mutant ROSA-CreERT2; Rb^{f/f}; p130^{f/f}; p107^{-/-} mice (RbTriKO) lacking Rb family proteins (Viatour, P. et al. Hematopoietic stem cell quiescence is maintained by compound contributions of the retinoblastoma gene family. Cell Stem Cell 3, 416-428 (2008)). After exposure to tamoxifen, β-cells in adult RbTriKO mice, lacking detectable Rb, p130 and p107, had increased Ezh2 protein and mRNA levels as compared to those from tamoxifen-exposed Rb^{f/f}, p130^{f/f} p107^{-/-} control littermates (FIG. 18a-c). Islet ChIP studies revealed increased E2f1 association, and reduced E2f4 association with Ezh2 cis-regulatory elements (FIG. 18d, e). Consistent with these findings, BrdU incorporation in RbTriKO β-cells was increased by 15-fold, culminating in a 400% increase of β-cell mass and hypoglycaemia that progressively worsened until death (FIG. 18f-h). Thus, Rb proteins constrain Ezh2 expression and adult islet cell growth.

### Example 6

PDGF Control of Human β-Cell Proliferation.

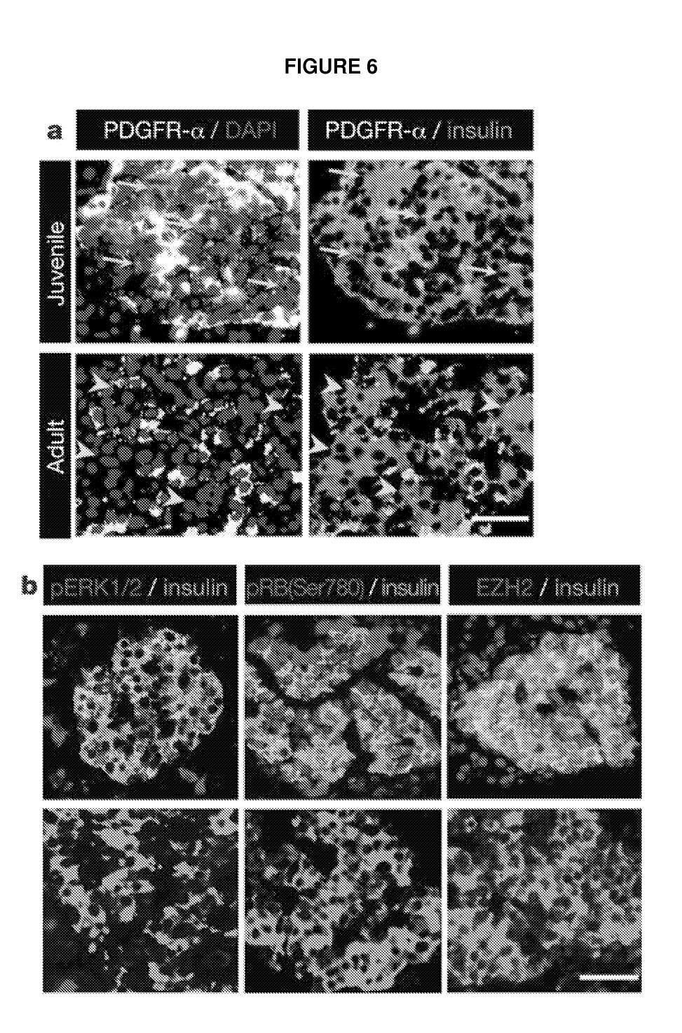

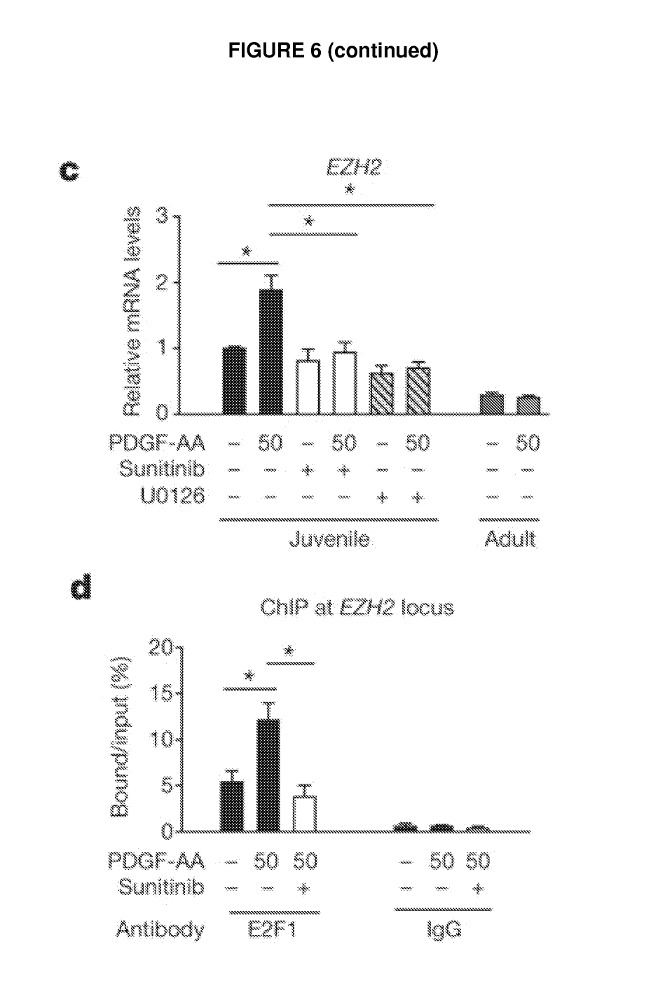

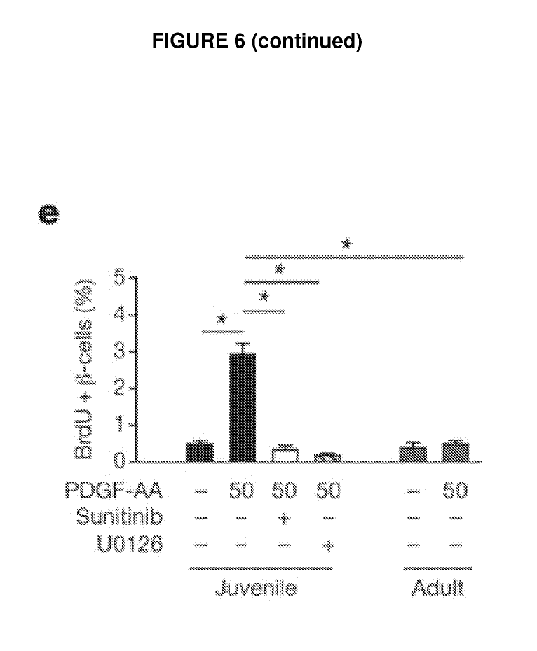

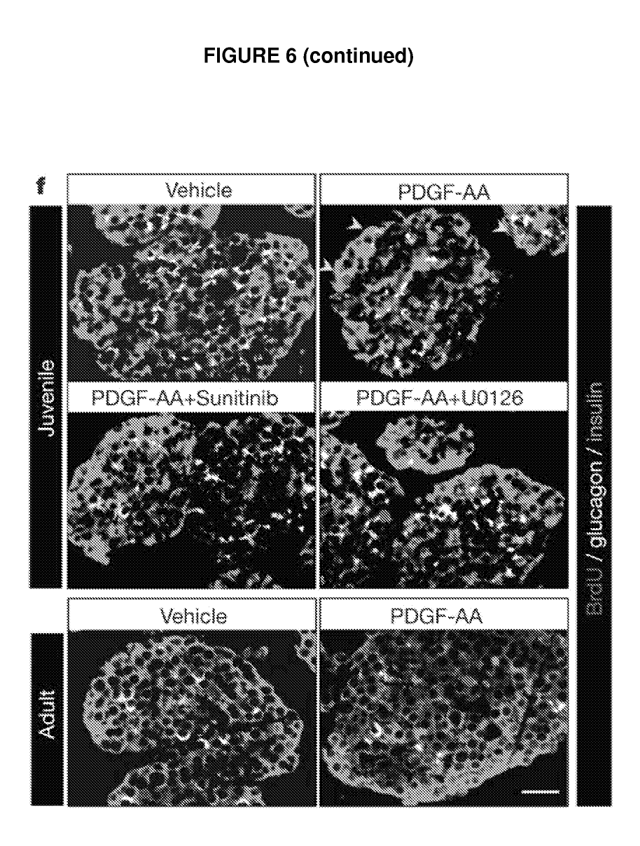

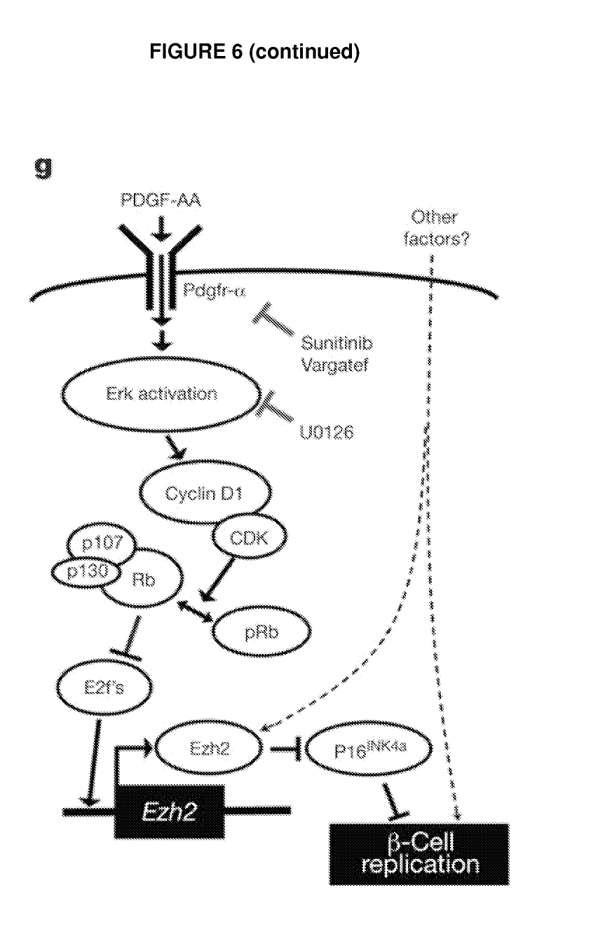

To investigate the relevance of our studies to human β-cells, we assessed PDGF signalling and competence in human islets. Like in juvenile mice, PDGFR-α protein and phospho-ERK1/2 levels were readily detected in islets from young human donors (FIG. 6a, b). We also detected abundant phospho-RB-Ser780 and EZH2 protein in β-cell nuclei from young islets (FIG. 6b). In contrast, PDGFR-α was only detected in non-β-cells of islets from adult human donors, with little to no detectable phospho-ERK1/2, phospho-RB or EZH2 in β-cells (FIG. 6a, b), suggesting that PDGF signalling attenuation is a conserved feature of ageing islet β-cells.

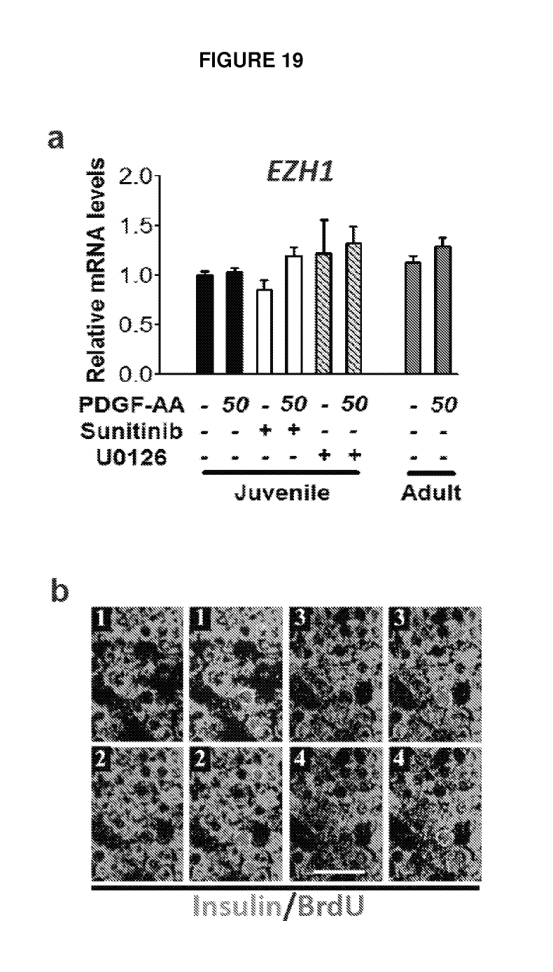

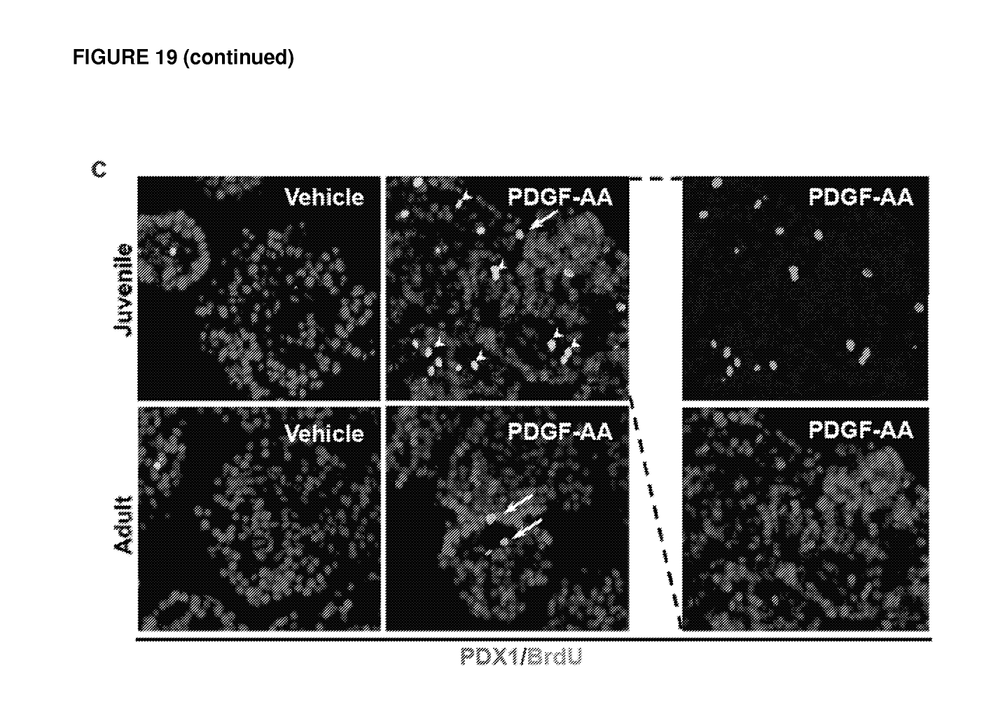

mRNA levels of EZH2, but not EZH1, were increased in juvenile human islets exposed to PDGF-AA, an effect blocked by Sunitinib or U0126 (FIG. 6c and FIG. 19a). In contrast, basal EZH2 mRNA levels were lower in adult islets and not induced by PDGF-AA (FIG. 6c). Similar to juvenile mouse islets with activated Pdgf signalling, ChIP studies demonstrated PDGF-AA-stimulated association of E2F1 with the human EZH2 locus, an effect blocked by Sunitinib (FIG. 6d). Thus, evolutionarily conserved elements of the PDGF signalling pathway govern EZH2 expression and β-cell cycle regulators in human islets. Proliferating BrdU+ cells in juvenile islets stimulated by PDGF-AA were predominantly β-cells, verified by immuno-localization of BrdU+ nuclei within insulin+ cells and PDX1+ nuclei with confocal microscopy (FIG. 6f and FIG. 19b, c). Compared to vehicle-exposed controls, juvenile islets exposed to PDGF-AA had a sixfold increase of β-cell BrdU incorporation, an effect eliminated by simultaneous exposure to Sunitinib or U0126 (FIG. 6e, f). In contrast, PDGF-AA did not alter β-cell proliferation in adult human islets (FIG. 6e, f). Thus, dynamic PDGF signalling competence may regulate declining β-cell proliferation in ageing human islets.

### Example 7

The question of what regulates loss of PDGFR expression in human and mouse islet β-cells with age, and whether modulation of the mechanisms underlying this age-dependent reduction of PDGFRα can be used to ‘restore’ PDGF signaling competence of adult β-cells to permit PDGFR-stimulated proliferation, was addressed. Prior studies of the PDGFRA promoter region in human, mouse and rat cells and tissues provide evidence for regulation by transcription factors including GATA4, PAX3, Pax1, C/EBP, Prx1 and Gli1 (Joosten et al 1998, 2002; Epstein et al 1998; Wang et al 1996; Fukuoka et al 1999; Xie et al 2001). However, the role of these factors in regulating age-dependent gene expression was unclear. DNA methylation at CpG ‘islands’ associated with gene regulatory elements is an established mechanism for repression and silencing of genes in aging (Shames et al 2007), and prior studies have implicated DNA methylation of the human PDGFRA locus in transformed human glial cells (Toepoel et al 2008). Several distinct haplotypes in the PDGFRA promoter region have been identified and correlated with expression levels in glioblastoma cells. These haplotype-specific levels of expression were correlated with epigenetic differences in methylation and histone acetylation associated with a CpG island found from −1,119 to −722 in the promoter region of human PDGFRA in glioblastoma cells. However, no study has demonstrated PDGFRA regulation by methylation in islet β cells.

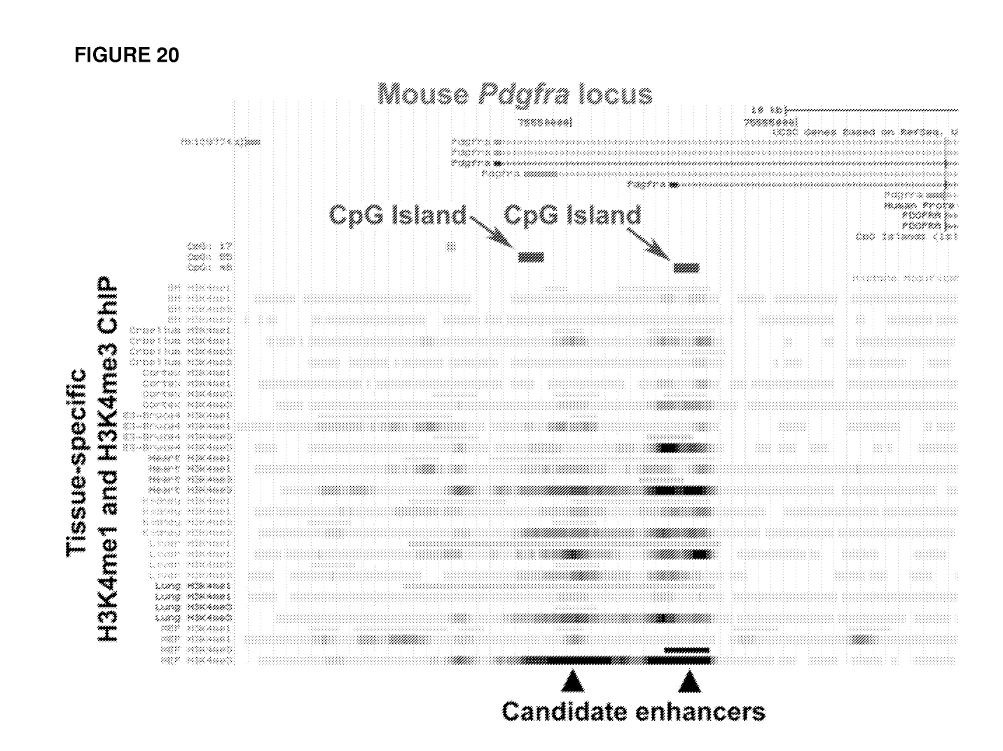

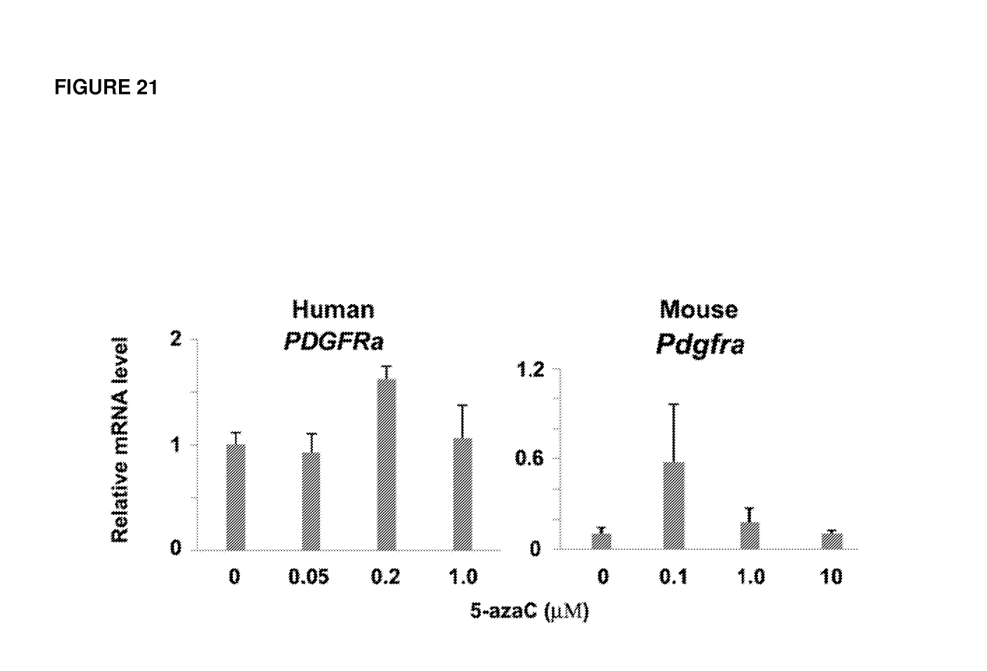

The CpG tool of the UCSC genome browser (v257) identifies two CpG islands upstream of the translational start site of the mouse Pdgfra gene, spanning the regions −9040/−9610 and −5662/−5054 (FIG. 20). The more 3′ promoter-proximal CpG island at −5662/−5054 aligns well with regions associated with histone H3K4me1 and H3K4me3, marks of enhancers or cis-regulatory elements, with lesser overlap in the more 5′ CpG island at −9040/−9610. There are also two CpG islands within the −3,600 to +118 region of the human PDGFRA promoter-proximal region, and evidence exists that DNA methylation within the most 3′ CpG island regulates PDGFRA expression (Toepoel et al 2008). As demonstrated in FIG. 21, dosage-dependent derepression of PDGFRA is observed in human and mouse islets exposed to the DNA methyltransferase (Dnmt) inhibitor 5-aza-cytidine (5-aza-C). These findings indicate that DNA methylation and associated chromatin remodeling regulates expression of islet β-cell PDGFRA.

### Example 8

Wnt Signaling Regulation of Age-Dependent Islet Pdgfra Expression.

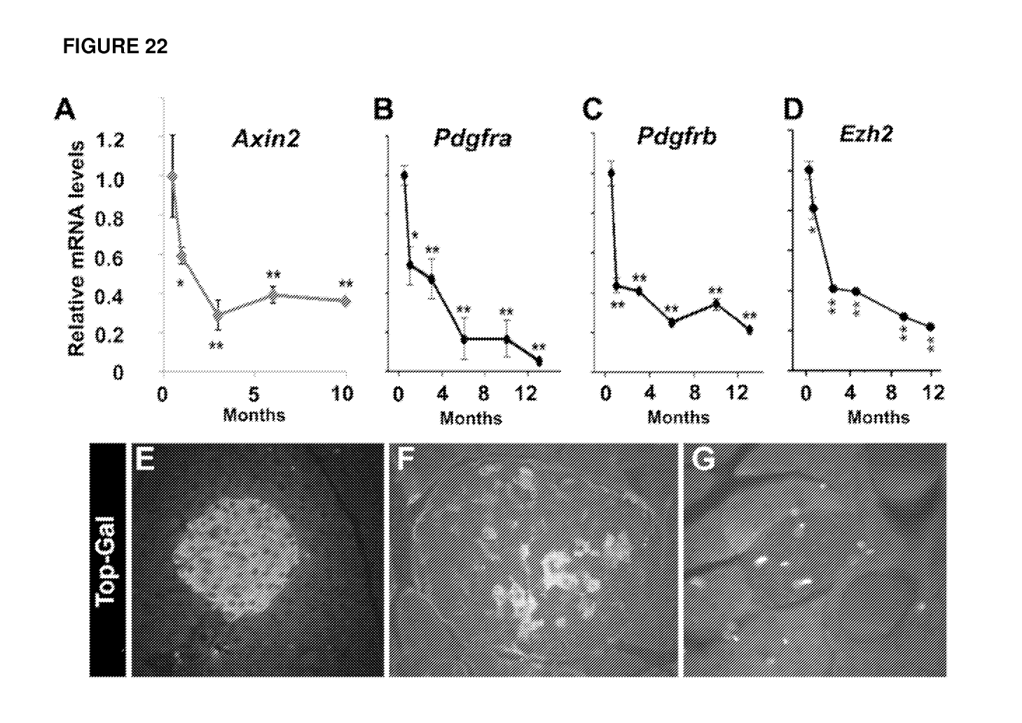

In diverse tissues, including the pancreas, Wnt signaling activates expression of Axin2, a negative regulator of the pathway. Studies of age-dependent Axin2 expression in staged mouse islets revealed abundant expression in neo-natal mice, followed by rapid reduction to nearly undetectable levels in adult islets (FIG. 22A). This spatially and temporally restricted expression pattern is reminiscent of declining Pdgfra, Pdgfrab, and Ezh2 levels and β-cell proliferation in animals (FIG. 22B-D, Chen et al 2009, 2011). Likewise, in mice harboring a ‘Top-Gal’ synthetic reporter gene of Wnt signaling activity, β-galactosidase reporter expression declines in islet β-cells with age (FIG. 22E-G). Thus, changes in Wnt signaling underlie age-dependent reductions of Pdgfr expression and β-cell PDGF signaling competence in β-cells. Consistent with this, culture of isolated adult mouse islets (4-5 months of age) with purified Wnt3a or the Wnt agonist R-Spondin increased Pdgfra mRNA expression.

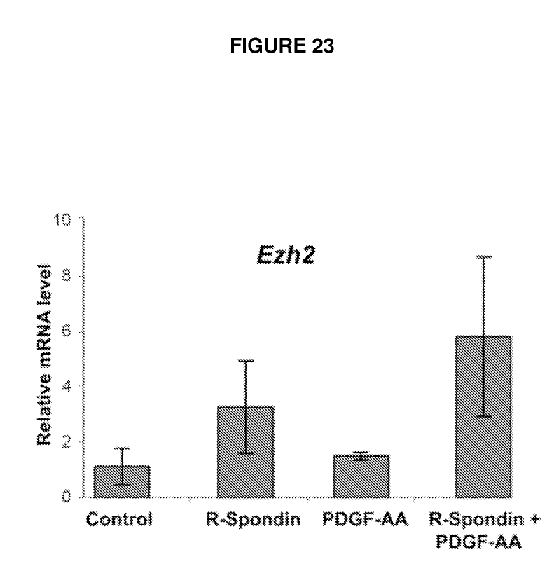

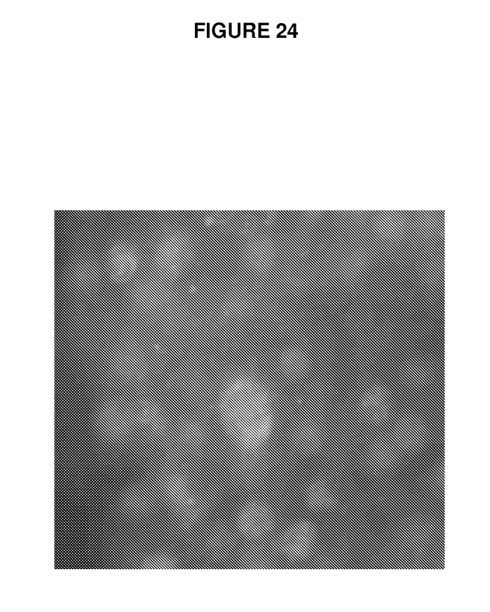

As described above, Pdgfra signaling induced Ezh2 expression in islets. To assess the function of Pdgfra induced by R-Spondin, R-Spondin was added to adult mouse islets, followed by addition of purified PDGF-AA (100 ng/ml), and Ezh2 mRNA levels were then measured. As shown in FIG. 23, R-Spondin alone modestly induced Ezh2, and the combination of R-Spondin followed by PDGF-AA exposure led to the best induction of Ezh2 mRNA. As shown in FIG. 24, immunohistochemistry revealed expression of Pdgfrα protein in adult islet PDX1+ β-cells exposed to R-Spondin. Similar staining was observed in islet INSULIN+ cells.

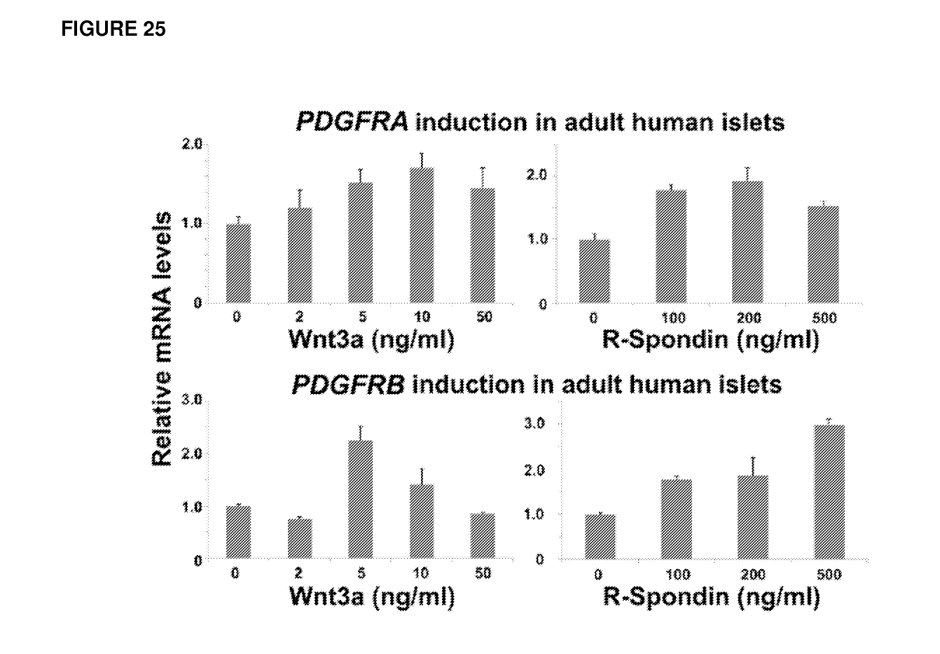

As shown in FIG. 25, similar results are observed with cultured human adult islets (n=3). mRNA levels encoding PDGFR α and PDGFR β are increased in adult human islets (mean age >40 years) exposed for two days either to purified Wnt3a or R-Spondin. Induction of PDGFRA and PDGFRB mRNA depends on the dose of Wnt3a or R-Spondin in these cultures.

### Example 9

Assessment of Human β-Cell Replication in Transplanted Islets.

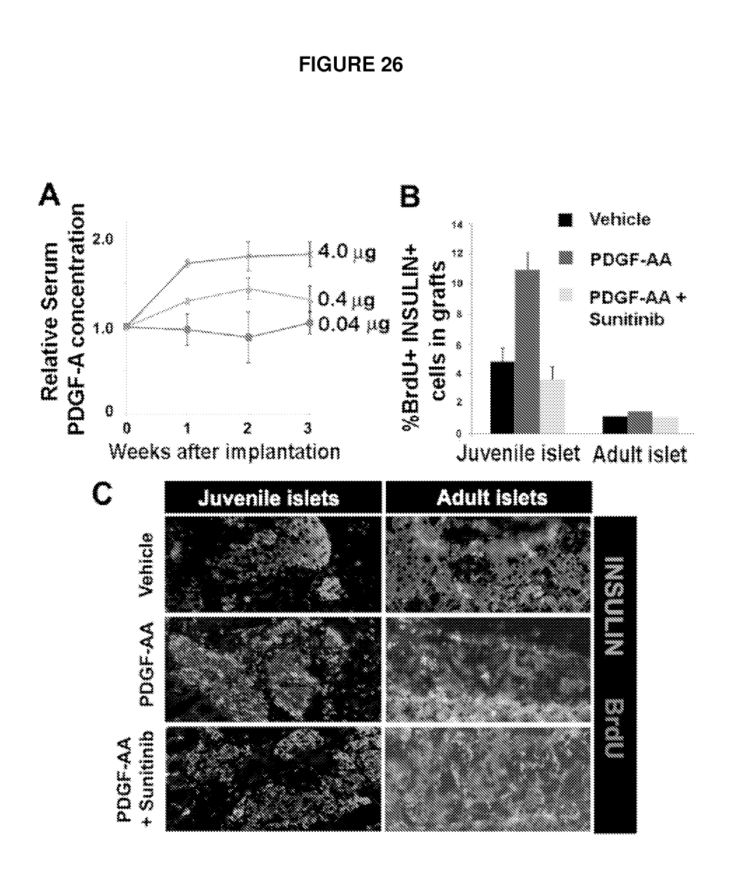

Exposure of islets from young human donors to purified PDGF-AA stimulates ERK-dependent signaling pathways culminating in enhanced β-cell proliferation. However, these studies have not yet shown if PDGF-AA activation is sufficient to drive regeneration of functional β-cell mass in vivo. To assess this question, equivalent numbers of size-matched islets from young (ages 1-10 years) and control adult donors (age >20 years) were transplanted into NOD-scid mice. Quantification of PDGF-AA by ELISA demonstrated increased serum PDGF-AA levels in control NOD-scid mice implanted with a subcutaneous osmotic pump primed with 4 micrograms (μ}g) of purified PDGF-AA (FIG. 26A).

PDGF-AA infusion stimulated β-cell expansion in human islet grafts. A 200% increase of INSULIN⁺ cell BrdU incorporation in grafts from young human donors (n=3 independent juvenile donors, three week PDGF-AA infusion; FIG. 26B-C). By contrast, we observed no increase of BrdU incorporation by INSULIN⁺ cells in adult islet grafts (n=2), as expected based on our prior observation that adult β-cells no longer express Pdgf receptor α or Pdgf receptor β. Promoting the expression of Pdgf receptor on adult islet β-cells, e.g. by promoting Wnt signaling and/or preventing DNA methylatransferase activity, will render these adult islet β-cells sensitive to PGF-AA infusion.

### DISCUSSION

The elucidation and control of mechanisms governing pancreatic β-cell proliferation will transform treatments for diseases like diabetes. However, prior attempts to expand islets by modulating extrinsic or intrinsic growth regulators have been bedevilled by limited proliferation with loss of defining β-cell features (Vasavada et al. Targeted expression of placental lactogen in the β-cells of transgenic mice results in β-cell proliferation, islet mass augmentation, and hypoglycemia. J. Biol. Chem. 275, 15399-15406 (2000); Garcia-Ocana et al. Hepatocyte growth factor overexpression in the islet of transgenic mice increases β-cell proliferation, enhances islet mass, and induces mild hypoglycemia. J. Biol. Chem. 275, 1226-1232 (2000)), indicating disruption of mechanisms that preserve fate in proliferating β-cells. The maintenance of cell fate and function in proliferating cells is the essence of epigenetic regulation. Here the principal elements of a native signalling pathway regulating Ezh2, an essential epigenetic regulator of β-cell proliferation, were identified. It was found that β-cell Pdgfr signalling was required for Ezh2 induction, to sustain both physiological and regenerative β-cell proliferation.

During islet β-cell regeneration studies herein and in maternal islets in pregnancy, a twofold increase of islet Ezh2 mRNA levels is observed. However, pathological loss of all Rb family proteins in RbTriKO islets led to a fourfold increase in Ezh2 mRNA, and a 15-fold increase in β-cell proliferation. RbTriKO mice became hypoglycaemic and developed other islet and metabolic phenotypes reminiscent of insulinoma pathogenesis. Thus, the physiological expansion of β-cells maintaining regulated function may require mechanisms limiting Ezh2 induction. Compared with β-cells from βEzh2KO mice, we also observed a modest enhancement of proliferation and expansion after PDGFR-α activation in β-cells lacking Ezh2. Insulin or prolactin, two mitogens capable of promoting β-cell proliferation (Heit et al., Intrinsic regulators of pancreatic β-cell proliferation. Annu. Rev. Cell Dev. Biol. 22, 311-338 (2006); Vasavada et al. Growth factors and β-cell replication. Int. J. Biochem. Cell Biol. 38, 931-950 (2006); Brelje, T. C., et al. Regulation of islet β-cell proliferation by prolactin in rat islets. Diabetes 43, 263-273 (1994)), did not induce β-cell Ezh2 expression in cultured islets. Thus, the findings here support the view that Ezh2-independent mechanisms regulate β-cell proliferation. Likewise, decline of β-cell proliferation in ageing βPDGFRαTg mice suggests that PDGF-independent pathways restrict β-cell expansion.

The roles of Pdgf signalling in islet β-cell growth and proliferation were previously not established. Here it is shown that loss of β-cell Pdgfr-α signalling accelerated β-cell replication failure both in juvenile mice and in adult mice after conditional β-cell ablation. In vivo activation of β-cell PDGFR-α signalling increased Ezh2 expression and mitigated age-dependent decline of β-cell proliferation. Crucially, conditional Ezh2 inactivation in β-cells with activated PDGFR-α signalling prevented these changes. Thus, the conditional loss- and gain-of-function studies presented herein reveal how Pdgf signalling regulates age-dependent physiological β-cell proliferation and expansion. This work also suggests that Mapk/Erk regulation links Pdgfr activation to Ezh2 induction and β-cell proliferation.

Although the mechanisms underlying pancreatic β-cell expansion in physiological settings may differ between species (Butler, A. E. et al. Adaptive changes in pancreatic β-cell fractional area and β-cell turnover in human pregnancy. Diabetologia 53, 2167-2176 (2010); Rieck, S. & Kaestner, K. H. Expansion of β-cell mass in response to pregnancy. Trends Endocrinol. Metab. 21, 151-158 (2010)), age-dependent restriction of β-cell proliferation is a conserved feature in mice and humans (Teta et al. Very slow turnover of β-cells in aged adult mice. Diabetes 54, 2557-2567 (2005); Meier et al. β-Cell replication is the primary mechanism subserving the postnatal expansion of β-cell mass in humans. Diabetes 57, 1584-1594 (2008)). Here, unprecedented evidence is provided that a conserved signalling pathway restricts β-cell proliferation in mice and humans. β-cell competence for signalling factors like PDGF can underlie dynamic islet responses to mitogens. Such age-dependent signalling competence explains earlier findings of inconsistent human β-cell responses to other mitogens (Beattie et al. A novel approach to increase human islet cell mass while preserving β-cell function. Diabetes 51, 3435-3439 (2002); Parnaud et al. Proliferation of sorted human and rat β-cells. Diabetologia 51, 91-100 (2008)). PDGF ligands are measurable in serum, and regulation of such circulating signalling agonists may provide an additional level of growth control in competent islet β-cells. Prior studies correlated increased PDGFR and EZH2 expression in human endocrine neoplasias (Ebert, M. et al. Induction of platelet-derived growth factor A and B chains and over-expression of their receptors in human pancreatic cancer. Int. J. Cancer 62, 529-535 (1995)), and disrupted PDGF signalling in β-cells of humans with type 2 diabetes (Nyblom, H. K. et al. Apoptotic, regenerative, and immune-related signaling in human islets from type 2 diabetes individuals. J. Proteome Res. 8, 5650-5656 (2009)). Thus, the demonstration herein that PDGF signalling regulates EZH2, CDKN2A and β-cell proliferation in human islets combines several observations into a new molecular model for physiological and pathological β-cell growth. Regulation of β-cell expression of PDGF signalling regulators like PDGFR-α will be useful for modulating human β-cell growth and function in diabetes and cancer.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of promoting β cell proliferation, comprising
contacting the β cell with an agents that promotes PDGFR signaling in an amount effective to promote β cell proliferation.

2. The method according to claim 1, wherein the agent promotes PDGFR activity.

3. The method according to claim 2, wherein the agent is a PDGF.

4. The method according to claim 2, further comprising contacting the cell with an agent that promotes PDGFR expression.

5. The method according to claim 4, wherein the agent that promotes PDGFR expression promotes Wnt signaling.

6. The method according to claim 4, wherein the agent that promotes PDGFR expression inhibits DNA methylation.

7. The method according to claim 4, wherein the PDGFR is PDGFRα.

8. The method according to claim 1, wherein the method is in vitro.

9. The method according to claim 8, wherein the method further comprises transplanting the cells into an individual with diabetes.

10. The method according to claim 1, wherein the method is in vivo.

11. The method according to claim 10, wherein the method is in an individual with diabetes.

12. A method of inhibiting β cell proliferation, comprising
contacting the β cell with an agents that inhibits PDGFR signaling in an amount effective to inhibit β cell proliferation.

13. The method according to claim 12, wherein the agent inhibits the expression of PDGFR.

14. The method according to claim 13, wherein the agent inhibits Wnt signaling.

15. The method according to claim 12, wherein the agent inhibits the binding of PDGF to PDGFR.

16. The method according to claim 12, wherein the agent inhibits the kinase activity of PDGFR or a downstream protein.

17. The method according to claim 12, wherein the PDGFR is PDGFRα.

18. The method according to claim 12, wherein the method is in vivo.

19. The method according to claim 18, wherein the method is in an individual with insulinoma, mixed endocrine tumor, or an acquired state of β cell overgrowth.

20. A method of screening a candidate agent for the ability to modulate β cell proliferation, comprising:
contacting a PDGFRα-expressing β cell with a candidate agent,
comparing the activity of PDGFRα in the β cell contacted with the agent to the PDGFRα activity in a cell not contacted with the candidate agent,
wherein a candidate agent that modulates the activity of PDGFRα is an agent that that modulates β cell proliferation.


SEQUENCE LISTING (42 sequences)

SEQ ID NO: 1 - DNA, 25 bp, Artificial Sequence
  synthetic oligonucleotide
gacaaatacatgtgcagctttctgt

SEQ ID NO: 2 - DNA, 25 bp, Artificial Sequence
  synthetic oligonucleotide
caacttgaacaatgattttgtggtg

SEQ ID NO: 3 - DNA, 17 bp, Artificial Sequence
  synthetic oligonucleotide
gccctttcgggttgcat

SEQ ID NO: 4 - DNA, 22 bp, Artificial Sequence
  synthetic oligonucleotide
gagcttggctaacttgggagaa

SEQ ID NO: 5 - DNA, 25 bp, Artificial Sequence
  synthetic oligonucleotide
tccgtggatggaatcctggaaggat

SEQ ID NO: 6 - DNA, 20 bp, Artificial Sequence
  synthetic oligonucleotide
atgcagatgccccagagttc

SEQ ID NO: 7 - DNA, 18 bp, Artificial Sequence
  synthetic oligonucleotide
gcagctaccgcagcatga

SEQ ID NO: 8 - DNA, 26 bp, Artificial Sequence
  synthetic oligonucleotide
cgagagctacacgctggagggaaatg

SEQ ID NO: 9 - DNA, 22 bp, Artificial Sequence
  synthetic oligonucleotide
aaggcacatgctaaccaatgaa

SEQ ID NO: 10 - DNA, 25 bp, Artificial Sequence
  synthetic oligonucleotide
gcaactacagcctagagggagaagt

SEQ ID NO: 11 - DNA, 22 bp, Artificial Sequence
  synthetic oligonucleotide
tacactggctggcatgctcttt

SEQ ID NO: 12 - DNA, 20 bp, Artificial Sequence
  synthetic oligonucleotide
ctcttgcggcaagcaacata

SEQ ID NO: 13 - DNA, 21 bp, Artificial Sequence
  synthetic oligonucleotide
gtaccccgattcaggtgatga

SEQ ID NO: 14 - DNA, 23 bp, Artificial Sequence
  synthetic oligonucleotide
cgttcacgtagcagctcttctgc

SEQ ID NO: 15 - DNA, 22 bp, Artificial Sequence
  synthetic oligonucleotide
cagttcgaatctgcaccgtagt

SEQ ID NO: 16 - DNA, 25 bp, Artificial Sequence
  synthetic oligonucleotide
tgaggctagagaggatcttgagaag

SEQ ID NO: 17 - DNA, 21 bp, Artificial Sequence
  synthetic oligonucleotide
ccggaatcctggaccaggtga

SEQ ID NO: 18 - DNA, 19 bp, Artificial Sequence
  synthetic oligonucleotide
cgtgaacgttgcccatcat

SEQ ID NO: 19 - DNA, 20 bp, Artificial Sequence
  synthetic oligonucleotide
tgctggaccaaacacaaacg

SEQ ID NO: 20 - DNA, 23 bp, Artificial Sequence
  synthetic oligonucleotide
ttcccagttttttatctgcactg

SEQ ID NO: 21 - DNA, 21 bp, Artificial Sequence
  synthetic oligonucleotide
tgcttgccatccagccattca

SEQ ID NO: 22 - DNA, 19 bp, Artificial Sequence
  synthetic oligonucleotide
ttgccaagagagccatcca

SEQ ID NO: 23 - DNA, 23 bp, Artificial Sequence
  synthetic oligonucleotide
actggcgaagagctgttttttga

SEQ ID NO: 24 - DNA, 21 bp, Artificial Sequence
  synthetic oligonucleotide
gcatcagcctggctgtatctg

SEQ ID NO: 25 - DNA, 24 bp, Artificial Sequence
  synthetic oligonucleotide
ataagggttcctgctttcacagaa

SEQ ID NO: 26 - DNA, 23 bp, Artificial Sequence
  synthetic oligonucleotide
tccagggtttatgtgtcagggtg

SEQ ID NO: 27 - DNA, 21 bp, Artificial Sequence
  synthetic oligonucleotide
gccattatggcgtgtgaagtc

SEQ ID NO: 28 - DNA, 21 bp, Artificial Sequence
  synthetic oligonucleotide
ttaccaacccgagttttgaac

SEQ ID NO: 29 - DNA, 20 bp, Artificial Sequence
  synthetic oligonucleotide
cgtctcctcccctccccctc

SEQ ID NO: 30 - DNA, 17 bp, Artificial Sequence
  synthetic oligonucleotide
gcgctgagtggttctcg

SEQ ID NO: 31 - DNA, 20 bp, Artificial Sequence
  synthetic oligonucleotide
ataaaagcgatggcgattgg

SEQ ID NO: 32 - DNA, 17 bp, Artificial Sequence
  synthetic oligonucleotide
ttgctgcgtttggcgct

SEQ ID NO: 33 - DNA, 21 bp, Artificial Sequence
  synthetic oligonucleotide
gactccactgccttcgatgtc

SEQ ID NO: 34 - DNA, 20 bp, Artificial Sequence
  synthetic oligonucleotide
aacgccttttcagtttcagg

SEQ ID NO: 35 - DNA, 30 bp, Artificial Sequence
  synthetic oligonucleotide
tgggtttttaatggcacataaaacatttga

SEQ ID NO: 36 - DNA, 24 bp, Artificial Sequence
  synthetic oligonucleotide
aaactacattcaactgtgacagca

SEQ ID NO: 37 - DNA, 17 bp, Artificial Sequence
  synthetic oligonucleotide
ccgccgttccgaaagtt

SEQ ID NO: 38 - DNA, 21 bp, Artificial Sequence
  synthetic oligonucleotide
ccttttatggctcgagtggcc

SEQ ID NO: 39 - DNA, 18 bp, Artificial Sequence
  synthetic oligonucleotide
gccgccgggttttatagg

SEQ ID NO: 40 - DNA, 16 bp, Artificial Sequence
  synthetic oligonucleotide
ggccctgtgattggac

SEQ ID NO: 41 - DNA, 27 bp, Artificial Sequence
  synthetic oligonucleotide
caataaaagcgatggcgattgggctgc

SEQ ID NO: 42 - DNA, 16 bp, Artificial Sequence
  synthetic oligonucleotide
ggaccgagcgccaaac